(19) United States
(12) Patent Application Publication
(10) Pub. No.: US 2022/0298252 A1
(43) Pub. Date: Sep. 22, 2022

(54) METHODS OF TREATING CANCER USING TNFRSF25 ANTIBODIES

(71) Applicant: Pelican Therapeutics, Inc., Durham, NC (US)

(72) Inventors: Matthew M. SEAVEY, Durham, NC (US); Jeff T. HUTCHINS, Durham, NC (US); Rahul R. JASUJA, Durham, NC (US)

(21) Appl. No.: 17/637,686

(22) PCT Filed: Aug. 27, 2020
(86) PCT No.: PCT/US20/48195

§ 371 Date: Feb. 23, 2022

Related U.S. Application Data
(60) Provisional application No. 62/932,028, filed on Nov. 7, 2019.
(60) Provisional application No. 62/903,363, filed on Sep. 20, 2019.
(60) Provisional application No. 62/894,095, filed on Aug. 30, 2019.

(51) Int. Cl. C07K 16/28; A61P 35/00; A61K 39/39

(52) U.S. Cl. CPC C07K 16/2878; C07K 16/2818; A61P 35/00; A61K 39/39; C07K 2317/75; C07K 2317/21; A61K 2039/507

(57) ABSTRACT

The present disclosure is directed to a method of treating cancer using a TNFRSF25 agonistic antibody or antigen binding fragment thereof and their combinations with additional therapies such as cancer vaccines and/or checkpoint inhibitors.

### CROSS-REFERENCE TO RELATED APPLICATIONS

This application claims priority to and benefit of U.S. Provisional Patent Application No. 62/894,095 filed on Aug. 30, 2019, U.S. Provisional Patent Application No. 62/903,363 filed on Sep. 20, 2019, and U.S. Provisional Patent Application No. 62/932,028 filed on Nov. 7, 2019, the contents of which are hereby incorporated by reference in their entireties.

### FIELD OF THE DISCLOSURE

The disclosure is directed to methods of treating cancer using anti-TNFRSF25 agonistic antibodies, e.g., in selected patient populations and/or in combination with second therapies.

### SEQUENCE LISTING

This application contains a Sequence Listing which has been submitted in ASCII format via EFS-Web and is hereby incorporated by reference in its entirety. Said ASCII copy, created on Aug. 18, 2020, is named PEL-015PC_119925-5015 Sequence Listing_ST25.txt and is 4,364 bytes in size.

### BACKGROUND

Cancer, a disease that arises from a prolonged period of genetic instability that extends the lifespan of a normal cell, is a significant health problem worldwide. Despite recent advances that have been made in detection and therapy of cancer, no vaccine or other universally successful method for prevention or treatment is currently available. Current therapies, which are generally based on a combination of immunotherapy, chemotherapy, surgery and radiation, continue to prove inadequate in many patients. Specifically, in the case of immunotherapy, while present therapies have shown some success, a challenge remains in identifying patients that are likely to respond. Such information would help oncologists select the treatment that is most likely to succeed, for instance with regard to combinations therapies. Further, patient screening would assist in determining, during the therapy, when time is of the essence, whether a desired immunological effect is being obtained. Such information would not only allow real time improvement of a therapy but also, in the case of non-responders, spare a patient from agents that will not provide benefits.

Accordingly, there exists a need in the art for improved methods for treating cancers, e.g. by monitoring therapeutic effects and/or selection of combination agents. The present disclosure fulfills these needs and further provides other related advantages.

### SUMMARY

This disclosure is based, at least in part, on the discovery that a T cell modulatory effect resulting from administering an effective amount of a Tumor Necrosis Factor Receptor Superfamily Member 25 (TNFRSF25) agonistic antibody or antigen binding fragment thereof can be used for treating cancer. The T cell modulatory effect is assessed in a sample from a patient and may be used to administer to the patient a second therapy for cancer treatment of the patient. The second therapy can be selected and administered based on the T cell modulatory effect of the TNFRSF25 agonistic antibody. For example, in some embodiments, the second therapy comprises a gp96-Ig fusion protein lacking the gp96 KDEL (SEQ ID NO: 10) sequence (i.e., HS-110), alone or in combination with OX40L-Ig (HS-130).

Accordingly, in one aspect, the disclosure provides a method for treating cancer, comprising administering an effective amount of an TNFRSF25 agonistic antibody or antigen binding fragment thereof to a patient in need thereof, assaying a sample from the patient for a T cell modulatory effect, and administering a second therapy to the patient based on the results of the assaying. The second therapy can be a checkpoint inhibitor, radiation therapy, chemotherapy, further administration of the TNFRSF25 agonistic antibody or antigen binding fragment thereof, a biological adjuvant (e.g., secretable vaccine protein such as a gp96-Ig fusion protein), or any combination thereof. In some embodiments, the second therapy comprises a gp96-Ig fusion protein lacking the gp96 KDEL (SEQ ID NO: 10) sequence (i.e., HS-110). In some embodiments, the second therapy comprises HS-110 in combination with OX40L-Ig (HS-130).

In some embodiments, the T cell modulatory effect is an expansion of CD4+ T cells and CD8+ T cells. In some embodiments, the expansion comprises an expansion sufficient to treat cancer in the patient, for example, sufficient to lead to an increase in a number of tumor-infiltrating lymphocytes in the patient, and/or a decrease in at least one of a tumor size and a tumor growth rate in the patient.

In some embodiments, the expansion of CD4+ T cells and CD8+ T cells may provide a certain ratio of CD4+ T cells to CD8+ T cell. For example, the expansion may provide a ratio of CD4+ T cells to CD8+ T cells of about 1:1. In some embodiments, the expansion may provide a differential ratio of CD4+ T cells to CD8+ T cells.

In some embodiments, the T cell modulatory effect is a differential expansion of effector memory and central memory T cells. For example, in some embodiments, the expansion provides an increase in a ratio of CD4⁺ central memory T cells to CD4+ effector memory T cells. As another example, the expansion provides an increase in a ratio of CD8+ effector memory to CD8+ central memory T cells.

In some embodiments, the second therapy is a checkpoint inhibitor. A checkpoint inhibitor may be an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2. For example, in some embodiments, the agent that targets PD-1 is an antibody or antibody format specific for PD-1, optionally selected from nivolumab, pembrolizumab, and pidilizumab. In other embodiments, the agent that targets PD-L1 is an antibody or antibody format specific for PD-L1, optionally selected from atezolizumab, avelumab, durvalumab, and BMS-936559.

In some embodiments, the agent that targets CTLA-4 is an antibody or antibody format specific for CTLA-4, optionally selected from ipilimumab and tremelimumab.

In some embodiments, the second therapy further comprises radiation therapy or chemotherapy. In some embodiments, the second therapy is further administration of the TNFRSF25 agonistic antibody or antigen binding fragment thereof.

In some embodiments, a method for treating cancer is provided that comprises administering an effective amount of a TNFRSF25 agonistic antibody or antigen binding fragment thereof to a patient in need thereof, administering a biological adjuvant to the patient, and administering a checkpoint inhibitor molecule to the patient.

In some embodiments, a method for treating cancer is provided that comprises administering an effective amount of a TNFRSF25 agonistic antibody or antigen binding fragment thereof to a patient in need thereof and administering a biological adjuvant to the patient. The patient may be undergoing treatment with a checkpoint inhibitor molecule.

The biological adjuvant may comprise a secretable vaccine protein such as, for example, gp96. In some embodiments, secretable vaccine protein is a gp96-Ig fusion protein. Furthermore, in some embodiments, the gp96-Ig fusion protein lacks the gp96 KDEL (SEQ ID NO: 10) sequence. In some embodiments, the biological adjuvant further comprises a T cell costimulatory fusion protein which enhances activation of antigen-specific T cells. The T cell costimulatory fusion protein can be, for example, OX40L-Ig which can be administered in combination with the gp96-Ig fusion protein lacking the gp96 KDEL (SEQ ID NO: 10) sequence.

In some embodiments, the TNFRSF25 agonistic antibody or antigen binding fragment thereof comprises: (i) a heavy chain variable region comprising heavy chain CDR1, CDR2, and CDR3 sequences, wherein the heavy chain CDR1 sequence is GFTFSNHDLN (SEQ ID NO: 1) or a variant thereof, the heavy chain CDR2 sequence is YISSASGLISYADAVRG (SEQ ID NO: 2) or a variant thereof; and the heavy chain CDR3 sequence is DPAYTGLYALDF (SEQ ID NO: 3) or a variant thereof or DPPYSGLYALDF (SEQ ID NO: 4) or a variant thereof; and (ii) a light chain variable region comprising light chain CDR1, CDR2, and CDR3 sequences, wherein the light chain CDR1 sequence is TLSSELSWYTIV (SEQ ID NO: 5) or a variant thereof, the light chain CDR2 sequence is LKSDGSHSKGD (SEQ ID NO: 6) or a variant thereof, and the light chain CDR3 sequence is CGAGYTLAGQYGWV (SEQ ID NO: 7) or a variant thereof. In some embodiments, the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences. The variable region FW sequences may be human.

Furthermore, in some embodiments, the TNFRSF25 agonistic antibody or antigen binding fragment thereof further comprises human heavy chain and light chain constant regions. The constant regions may be selected from the group consisting of human IgG1, IgG2, IgG3, and IgG4. For example, the constant regions may be IgG1. As another example, the constant regions may be IgG4.

In some embodiments, the TNFRSF25 agonistic antibody or antigen binding fragment thereof comprises a heavy chain variable region of the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of about 85% to about 99% identity thereto. In some embodiments, the TNFRSF25 agonistic antibody or antigen binding fragment thereof comprises a light chain variable region of the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of about 85% to about 99% identity thereto.

In some embodiments, the second therapy is a biological adjuvant. The biological adjuvant may comprise a secretable vaccine protein such as, for example, gp96. In some embodiments, the secretable vaccine protein is a gp96-Ig fusion protein. The gp96-Ig fusion protein may lack the gp96 KDEL (SEQ ID NO: 10) sequence such that the gp96-Ig fusion is Viagenpumatucel-L (HS-110).

In some embodiments, the Ig tag in the gp96-Ig fusion protein comprises the Fc region of human IgG1, IgG2, IgG3, IgG4, IgM, IgA, or IgE.

Furthermore, in some embodiments, the biological adjuvant further comprises a T cell costimulatory fusion protein which enhances activation of antigen-specific T cells. The T cell costimulatory fusion protein may be selected from OX40L-Ig, or a portion thereof that binds to OX40, ICOSL-Ig, or a portion thereof that binds to ICOS, 4 1BBL-Ig, or a portion thereof that binds to 4-1BBR, TL1A-Ig, or a portion thereof that binds to TNFRSF25, GITRL-Ig, or a portion thereof that binds to GITR, CD40L-Ig, or a portion thereof that binds to CD40, and, CD70-Ig, or a portion thereof that binds to CD27. In some embodiments, the T cell costimulatory fusion protein is an Ig fusion protein. The Ig tag in the T cell costimulatory fusion protein may comprise the Fc region of human IgG1, IgG2, IgG3, IgG4, IgM, IgA, or IgE.

In some embodiments, the T cell costimulatory fusion protein is OX40L-Ig (HS-130) administered in combination with the gp96-Ig fusion protein lacking the gp96 KDEL (SEQ ID NO: 10) sequence (i.e., HS-110). Also, in some embodiments, the second therapy can be HS-130 alone. In some embodiments, gp96-Ig and OX40L-Ig are secreted by separate cell lines (HS-110 and HS-130, respectively). In other embodiments, a cell line is used that secrets both gp96-Ig and OX40L-Ig.

In some embodiments, gp96-Ig, alone or in combination with OX40L-Ig, is administered to a patient in need thereof along with an effective amount of an TNFRSF25 agonistic antibody or antigen binding fragment thereof (e.g., PTX-35). In some embodiments, a checkpoint inhibitor can additionally be administered. The checkpoint inhibitor can be an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2. In some embodiments, the checkpoint inhibitor is an agent that targets PD-1. In some embodiments, gp96-Ig and OX40L-Ig are secreted by separate cell lines (HS-110 and HS-130, respectively), while in other embodiments the same cell line may secret both gp96-Ig and OX40L-Ig.

In some embodiments, the secretable vaccine protein and/or T cell costimulatory fusion protein are encoded on an expression vector. The expression vector may be incorporated into a human tumor cell which may be, in some embodiments, an irradiated or live and attenuated human tumor cell. For example, the human tumor cell may be a cell from an established NSCLC, bladder cancer, melanoma, ovarian cancer, renal cell carcinoma, prostate carcinoma, sarcoma, breast carcinoma, squamous cell carcinoma, head and neck carcinoma, hepatocellular carcinoma, pancreatic carcinoma, or colon carcinoma cell line.

In some embodiments, the sample from the patient is selected from a tissue biopsy, tumor biopsy, tumor resection, frozen tumor tissue specimen, lymph node, bone marrow, circulating tumor cells, cultured cells, a formalin-fixed paraffin embedded tumor tissue specimen, and combinations thereof. The biopsy may be selected from a core biopsy, needle biopsy, surgical biopsy, and an excisional biopsy. The assay may be a measurement of cytokine levels, cytokine secretion, surface markers, cytolytic protein secretion, and/or genomic profiles. For example, the assay may employ cytoplasmic dyes, optionally being carboxyfluorescein succinimidyl ester (CFSE). As another example, the assay may employ CFSE and measure cell proliferation. In some embodiments, the assay employs one or more of ELISPOT (enzyme-linked immunospot), intracellular cytokine staining (ICS), fluorescence-activated cell sorting, (FACS), microfluidics, PCR, and nucleic acid sequencing. In other embodiments, the assay employs analysis of surface markers such as CD45RA/RO isoforms and CCR7 or CD62L expression. As another example, the assay may be a measurement of cytokine levels and/or cytokine secretion, and the cytokine is selected from one or more of IFN-γ, TNF, and IL-2.

### BRIEF DESCRIPTION OF THE DRAWINGS

FIG. 1 is a line graph illustrating a percentage of CD4⁺FoxP3⁺ T-reg cells for various doses of PTX-35 combined in the study with non-human primates. The graph shows Mean±SEM; Mann-Whitney, non-parametric test performed for statistics as compared to baseline/day-0 values, *p<0.05, and ***p<0.001. Arrows indicate days of PTX-35 infusions (dose).

FIGS. 2A and 2B illustrate a percentage of activated T cells in treated non-human primates. FIG. 2A is a graph illustrating a percentage of recently activated T cells (% of CD45⁺CD3⁺CD69⁺ T cells), and FIG. 2B is a graph illustrating a percentage of all activated T cells (% of CD45⁺CD3⁺CD25⁺ T cells).

FIGS. 3A and 3B illustrate a percentage of CD4⁺ T-regs and endogenous activated CD4⁺ T cells in treated non-human primates. FIG. 3A is a graph illustrating percentage of CD4⁺ T-regs (% of CD45⁺FOXP3 T cells), and FIG. 3B is a graph illustrating percentage of activated CD4⁺ T cells (% of CD45⁺CD3⁺CD4⁺CD25⁺ T cells).

FIG. 4 is a line graph illustrating a percentage of activated CD8⁺ T cells (% of CD45⁺CD3⁺CD8⁺CD69⁺ T cells) over days 0-22.

FIGS. 5A-5C are line graphs illustrating a percentage of CD4⁺ T cells over days 0-22 in treated non-human primates. FIG. 5A shows a percentage of naïve CD4⁺ T cells (% of CD45⁺CD3⁺CD4⁺CD28⁺CD95⁺ naïve T cells), FIG. 5B shows a percentage of central memory CD4⁺ T cells (% of CD45⁺CD3⁺CD4⁺CD28⁺CD95⁺ CM (central memory) T cells), and FIG. 5C shows a percentage of effector memory CD4⁺ T cells (% of CD45⁺CD3⁺CD4⁺CD28⁺CD95⁺ EM (effector memory) T cells).

FIGS. 6A-6C are line graphs illustrating a percentage of CD8⁺ T cells over days 0-22. FIG. 6A shows a percentage of naïve CD8⁺ T cells (% of CD45⁺CD3⁺CD8⁺CD28⁺CD95⁺ naïve T cells), FIG. 6B shows a percentage of central memory CD8⁺ T cells (% of CD45⁺CD3⁺CD8⁺CD28⁺CD95⁺ CM (central memory) T cells), and FIG. 6C shows a percentage of effector memory CD8⁺ T cells (% of CD45⁺CD3⁺CD8⁺CD28⁺CD95⁺ EM (effector memory) T cells).

FIGS. 7A and 7B are line graphs illustrating effect of human PTX-35 costimulation of TCR-engaged effector cells after 72 hours of stimulation with plate-bound anti-CD3. FIG. 7A illustrates the proportion of CD4⁺ T cells that divided (% of proliferation within CD4+) vs concentration of anti-CD3 (in ng/mL), and FIG. 7B illustrates the proportion of CD8⁺ T cells that divided (% of proliferation within CD8+) vs concentration of anti-CD3 (in ng/mL). The graphs in FIGS. 7A and 7B show data for 100 ng/ml human PTX-35, vehicle and 100 ng/mL isotype control; the data is mean+SEM, and “***” denotes p<0.0001 by two-way ANOVA comparing PTX-35 to isotype control group.

FIG. 8 is a schematic diagram illustrating generation of a mouse-human surrogate antibody for PTX-35. Panel A shows the parental hamster antibody 4C12, panel B shows human PTX-35, and panel C shows surrogate mouse antibody, mPTX-35.

FIG. 9 are line graphs illustrating testing of surrogate mouse PTX-35 (mPTX-35) in human Jurkat cells expressing human DR3 (Jurkat DR-3 cells). NF-kB luciferase activity is shown, versus a concentration of the tested antibodies. Jurkat-DR3 cells were treated with three different lots of human PTX-35 (clinical lot A (blue line), clinical lot B (red line), and clinical lot C (green line)), two lots of 4C12 (RL #180618 (magenta line) and RL #181017 (maroon line)), mPTX-35 IgG1 (black line), and mPTX-35 IgG2a (brown line). The graphs for IgG1 and IgG2a (shown in dark blue and purple lines, respectively) overlap with the graphs indicating results for 4C12. The thin blue line shows PTX-15 (human TL1A-Ig) used as a positive control.

FIG. 10 is a non-limiting schematic illustrating a design of in vivo experiments using a radiation priming model for tumor treatment. On day 0, 4T1 tumor cells were injected in the fat pad of Balb/c mice; and, on days 7-13, mice were radiated with 5 Gy radiation for 6 days (5×6) amounting to a cumulative radiation dosage of 30 Gy to thereby generate metastatic models. Antibodies were administered at doses 100 μg/mouse.

FIG. 11 are line graphs illustrating results of the murine breast cancer model study of FIG. 10, where cumulative tumor growth curves (tumor volume (in mm³) vs days post-implantation) were followed over the course of 30 days between the following treatment groups: Radiation alone (5×6, black graph with circles), Radiation+Isotype (5×6+ISO, gray graph with circles), Radiation+TNFRSF25 (5×6+TNFRSF25, red graph with rhombuses). The Radiation+TNFRSF25 graph is below the graphs representing Radiation alone and Radiation+Isotype. For reference, on day 28, the tumor volume for Radiation+Isotype (5×6+ISO) is about 240 mm³, the tumor volume for Radiation alone (5×6) is about 180 mm³, and the tumor volume for Radiation+TNFRSF25 (5×6+TNFRSF25) is about 105 mm³. Arrows indicate the time when treatment was administered (radiation±agonist).

FIG. 12 are line graphs illustrating results of the mouse breast cancer model study of FIG. 10, where cumulative tumor growth curves (tumor volume (in mm³) vs days post-implantation) are followed over the course of 30 days between the following groups: Radiation alone (5×6, gray graph with circles), Radiation+Isotype (5×6+ISO, black graph with circles), Radiation+TNFRSF25 (5×6+TNFRSF25, red graph with rhombuses), Radiation+CTLA+TNFRSF25 (5×6+TNFRSF25+CTLA, green graph with squares), and Radiation+CTLA-4 (5×6+CTLA, blue graph with triangles) treatment groups. For reference, on day 28, the tumor volume for Radiation+Isotype (5×6+ISO) is about 240 mm³, the tumor volume for Radiation alone (5×6) is about 180 mm³, the tumor volume for Radiation+TNFRSF25 (5×6+TNFRSF25) is about 105 mm³, and the tumor volume for Radiation+CTLA+TNFRSF25 (5×6+TNFRSF25+CTLA) is about 40 mm³.

FIG. 13 are line graphs illustrating results of the mouse breast cancer model study of FIG. 10, where cumulative tumor growth curves (tumor volume (in mm³) vs days post-implantation) are followed over the course of 30 days between the following groups: Radiation alone (5×6, graph with squares), Radiation+Isotype (5×6+ISO, graph with circles), Radiation+TNFRSF25 (5×6+TNFRSF25, graph with rhombuses), Radiation+TNFRSF25+PD1 inhibitor (5×6+TNFRSF25+PD1, graph with up-pointing triangles), and Radiation+PD1 inhibitor (5×6+PD1, graph with down-pointing triangles) treatment groups. For reference, on day 28, the tumor volume for Radiation+Isotype (5×6+ISO) is about 280 mm³, the tumor volume for Radiation alone (5×6) is about 180 mm³, the tumor volume for Radiation+TNFRSF25 (5×6+TNFRSF25) is about 100 mm³, the tumor volume for Radiation+PD1 inhibitor (5×6+PD1) is about 90 mm³, and the tumor volume for Radiation+TNFRSF25+PD1 inhibitor (5×6+TNFRSF25+PD1) is about 55 mm³.

FIG. 14 is a non-limiting schematic illustrating a design of the study of CD8⁺ T cell expansion using a TNFRSF25 agonist, mPTX-35, in combination with a gp96-Ig-secreting cancer vaccine mHS-110 (depicted as “gp96”).

FIG. 15 shows anti-tumor CD8+ OT-1⁺ T cell expansion, in the peripheral blood with immunization with mHS-110 with different doses of mPTX-35 (the study of FIG. 14), in the analyzed groups: 10 mg/kg mPTX-35 (panel A), 100 ng mHS-110 (panel B), 100 ng mHS-110+0.1 mg/kg mPTX-35 (panel C), 100 ng mHS-110+1 mg/kg mPTX-35 (panel D), and 100 ng mHS-110+10 mg/kg mPTX-35 (panel E). Data is generated using flow cytometry gating (day 4), in the study of FIG. 14. Total live cells were gated by SSC (side scatter) and FSC (forward scatter) parameters, then cells were gated on CD3⁺ T-cell events, then gated as shown in FIG. 15 by eGFP⁺ OT-I⁺ CD8⁺ T cell events. The adoptively transferred T cells as shown in FIG. 15 during the peak expansion of day 4 in the peripheral blood.

FIG. 16 are line graphs illustrating a percentage of CD8+ OT-I⁺ T cells over time (0-52 days) in the study of CD8⁺ T cell expansion with mPTX-35+mHS-110 (the study of FIG. 14). Total OT-I cells as a percentage of CD8⁺ T-cells were gated and graphed. The graphs show mean±SEM for each group on each day by peripheral blood compartment.

FIG. 17 are bar graphs illustrating a percentage of CD8+ OT-I⁺ T cells on day 4 (panel A) and day 38 (panel B), in the study of FIGS. 14 and 16. Statistics was performed by Mann-Whitney, two-tailed, test; *p<0.05, **p<0.01, ***p<0.001, ns=p>0.05=not significant.

FIG. 18 are line graphs illustrating synergistic antigen-specific T cell expansion and tumor cell killing with mPTX-35 and mHS-110, as tumor growth kinetics for five groups: 100 ng mHS-110, 100 ng mHS-110+0.1 mg/kg mPTX-35, 100 ng mHS-110+1 mg/kg mPTX-35, 100 ng mHS-110+10 mg/kg mPTX-35, and 10 mg/kg mPTX-35 (the study of FIG. 14). Tumor size was measured by calipers.

FIG. 19 illustrates synergistic antigen-specific T cell expansion and tumor cell killing with mPTX-35 and mHS-110 in the murine model. Panel A shows tumor weight bar graphs and panel B shows tumor weight scatter plots illustrating the end tumor mass (day 52, the study of FIG. 14) for the five groups: 10 mg/kg mPTX-35, 100 ng mHS-110, 100 ng mHS-110+0.1 mg/kg mPTX-35, 100 ng mHS-110+1 mg/kg mPTX-35, and 100 ng mHS-110+10 mg/kg mPTX-35. Graphs show mean±SEM, statistical analysis performed was non-parametric t-test, Mann-Whitey, two tailed; *p<0.05, **p<0.01, ***p<0.001, ns=p>0.05=not significant.

FIG. 20 are bar graphs illustrating Tumor Infiltrating Lymphocytes (TILs) for endogenous T cells, on day 52 of the study of FIG. 14. TILs were extracted and analyzed by flow cytometry. Panel A shows percentage of CD3⁺CD8⁺ endogenous TILs, panel B shows percentage of CD3⁺CD4⁺ endogenous TILs. The graphs show mean±SEM, statistical analysis performed was non-parametric t-test, Mann-Whitey, two tailed; *p<0.05, **p<0.01, ***p<0.001, ns=p>0.05=not significant.

FIG. 21 is a non-limiting schematic illustrating a design of the study of CD8⁺ T cell expansion kinetics and tumor challenge with treatment using mHS-110 (gp96-Ig), mHS-130 (OX40L-Ig), and mPTX-35 (anti-TNFRSF25 mAb), or combinations thereof, in vivo.

FIG. 22 are graphs illustrating anti-tumor CD8+ OT-I, T cell expansion, in the peripheral blood with prime immunization of mHS-110 and mHS-130 with different doses of mPTX-35 in the study of FIG. 21. Total live cells were gated by side-scattered light (SSC) and forward-scattered light (FSC) parameters, then cells were gated on CD3⁺ T-cell events, and gated by eGFP⁺ OT-1+ CD8⁺ T cell events. The adoptively transferred T cells are shown during the peak expansion on day 5 in the peripheral blood.

FIG. 23 are graphs illustrating anti-tumor CD8+ OT-I, T cell expansion, in the peripheral blood with prime and boost immunization of mHS-110 and mHS-130 with different doses of mPTX-35 in the study of FIG. 21. Total live cells were gated by SSC and FSC parameters, then cells were gated on CD3⁺ T-cell events, and gated by eGFP+ OT-1+ CD8⁺ T cell events. The adoptively transferred T cells are shown during the peak expansion on day 19 after the first boost in the peripheral blood.

FIGS. 24A, 24B, and 24C are graphs illustrating expansion of total OT-1 cells as a percentage of gated CD8⁺ T cells in the study of FIG. 21. FIG. 24A shows T cell expansion for mHS-110 (100 ng), mHS-130 (100 ng), and 1 mg/kg PTX-35. FIG. 24B shows T cell expansion for mHS-110+mHS-130, mHS-130+mPTX-35 (1 mg/kg), and mHS-110+mPTX-35 (1 mg/kg). FIG. 24C shows T cell expansion for mHS-110+mHS-130+mPTX-35 (0.1 mg/kg), mHS-110+mHS-130+mPTX-35 (1 mg/kg), and mHS-110+mHS-130+mPTX-35 (10 mg/kg).

FIG. 25 is a bar chart illustrating percent of gated CD8⁺ OT-1⁺ exogenous T cells from peripheral blood on day 5 of the study of FIG. 21 after the primary immunization. The chart shows mean±SEM for each group by peripheral blood compartment. Statistics performed by Mann-Whitney, two-tailed, test. *p<0.05, **p<0.01, ***p<0.001, ns=p>0.05=not significant.

FIG. 26 is a bar chart illustrating percent of gated CD8⁺CD44⁺ OT-1⁺ exogenous T cells from peripheral blood on day 5 of the study of FIG. 21 after the primary immunization. The chart shows mean±SEM for each group by peripheral blood compartment. Statistics performed by Mann-Whitney, two-tailed, test. *p<0.05, **p<0.01, ***p<0.001, ns=p>0.05=not significant.

FIG. 27 is a bar chart illustrating percent of gated CD8⁺CD44⁺ endogenous T cells on day 5 of the study of FIG. 21. The chart shows mean±SEM for each group by peripheral blood compartment. Statistics performed by Mann-Whitney, two-tailed, test. *p<0.05, **p<0.01, ***p<0.001, ns=p>0.05=not significant.

FIG. 28 is a bar chart illustrating percent of gated CD8⁺ OT-1⁺ exogenous T cells on day 19 after the first boost immunization in the study of FIG. 21. The chart shows mean±SEM for each group by peripheral blood compartment. Statistics performed by Mann-Whitney, two-tailed, test. *p<0.05, **p<0.01, ***p<0.001, ns=p>0.05=not significant.

FIG. 29 is a bar chart illustrating percent of gated CD8⁺CD44⁺ OT-1⁺ exogenous T cells on day 19 of the study of FIG. 21. The chart shows mean±SEM for each group by peripheral blood compartment. Statistics performed by Mann-Whitney, two-tailed, test. *p<0.05, **p<0.01, ***p<0.001, ns=p>0.05=not significant.

FIG. 30 is a bar chart illustrating percent of gated CD8⁺CD44⁺ endogenous T-cells on day 19 of the study of FIG. 21. The chart shows mean±SEM for each group by peripheral blood compartment. Statistics performed by Mann-Whitney, two-tailed, test. *p<0.05, **p<0.01, ***p<0.001, ns=p>0.05=not significant.

FIG. 31 are graphs illustrating mean averaged mouse tumor volumes on day 4 (study day 32) through day 20 (study day 48, the end of the study) of the study of FIG. 21. The graphs show mean±SEM. Statistical analysis performed was 2-way ANOVA by GraphPad Prism software comparing all groups to individual controls. *p<0.05, **p<0.01, ***p<0.001, ns=p>0.05=not significant.

FIG. 32 is a non-limiting schematic illustrating a design of the study of combining PTX-35 with gp96-Ig (mHS-110)/OX40L-Ig (mHS-130). C57BL/6 mice (n=5 mice per group) were injected subcutaneously (S.C.) with 500,000 B16.F10 melanoma tumors and 3 days later adoptively transferred with one-million syngeneic OT-I transgenic CD8 T cells. Mice were subsequently treated on days 4 (gp96-Ig/OX40L-Ig+mPTX-35) and 18 (gp96-Ig/OX40L-Ig+mPTX-35, boost) post tumor inoculation and monitored for tumor growth inhibition (TGI).

FIG. 33 are representative Fluorescence-Activated Cell Sorting (FACS) plots illustrating the percentage of CD3+CD8+ OT-I⁺ T cells in the peripheral blood during the peak of CD8 T cell responses on day 4 post-treatment, in the study of FIG. 32.

FIG. 34 is a bar graph illustrating the percentage of CD3+CD8+ OT-I⁺ T cells in the peripheral blood during the peak of CD8 T cell responses on day 4 post-treatment. CD3+CD8+ OT-I⁺ T cells are presented as the mean±SEM of the experiment, in the study of FIG. 32.

FIG. 35 is a bar graph illustrating percentage of CD3+CD8+CD44⁺ T cells during the peak of CD8 T cell responses on day 4 post-treatment, presented as the mean±SEM of the experiment, in the study of FIG. 32.

FIG. 36 is a bar graph illustrating percentage of CD3+CD8+ OT-I⁺ CD44⁺ T cells during the peak of CD8 T cell responses on day 4 post-treatment, presented as the mean±SEM of the experiment, in the study of FIG. 32.

FIG. 37 are representative FACS plots illustrating the percentage of CD3+CD8+ OT-I⁺ T cells in the spleen on day 17 post-treatment, in the study of FIG. 32.

FIG. 38 is a bar graph illustrating the percentage of CD3+CD8+ OT-I⁺ T cells in the spleen on day 17 post-treatment, presented as the mean±SEM of the experiment, in the study of FIG. 32.

FIG. 39 is a bar graph illustrating the percentage of CD3+CD8+ OT-I⁺ effector memory cells (% CD3+CD8+ OT-I⁺ CD44+CD62L− T cells) in the spleen on day 17 post-treatment, presented as the mean±SEM of the experiment, in the study of FIG. 32.

FIG. 40 are representative FACS plots illustrating the percentage of CD3+CD8+ OT-I⁺ T cells in the tumor on day 17 post-treatment, in the study of FIG. 32.

FIG. 41 is a bar graph illustrating the percentage of CD3+CD8+ OT-I⁺ T cells in the tumor on day 17 post-treatment, presented as the mean±SEM of the experiment, in the study of FIG. 32.

FIG. 42 are graphs illustrating average tumor sizes, in diameter (mm²), over days 2-21 of the study of FIG. 32. The tumor size was measured every 2 days with a caliper, starting on day 2, and calculated using the formula (L×S), where L is the largest diameter of tumor and S is the smallest diameter of tumor.

FIGS. 43A and 43B are graphs illustrating tumor sizes, in diameter (mm²), over days 2-21 of the study of FIG. 32. The tumor size was measured every 2 days with a caliper, starting on day 2, for each mouse (n=5) in each of the groups. FIG. 43A illustrates the tumor diameter for vehicle (PBS), 1 mg/kg PTX-35, and mHS-110+mHS-130. FIG. 43B illustrates the tumor diameter for mHS-110+mHS-130+0.1 mg/kg PTX-35, mHS-110+mHS-130+1 mg/kg PTX-35 (mPTX-35med), and mHS-110+mHS-130+10 mg/kg PTX-35.

FIG. 44 are scatter plots illustrating tumor mass (in grams) for each mouse in each of the groups (n=5), on day 21 post-tumor inoculation, in the study of FIG. 32.

FIG. 45 is a non-limiting schematic illustrating a design of the study of effect of combination of checkpoint inhibition (αPD1) with gp96-Ig and/or PTX-35 (anti-TNFRSF25 mAb). C57BL/6 mice (n=5 mice per group) were injected subcutaneously (S.C.) with 500,000 B16.F10 melanoma tumors and 2 days later adoptively transferred with one-million syngeneic OT-I transgenic CD8 T cells. Mice were subsequently treated on days 3 (gp96-Ig/mPTX-35+anti-PD1) and 17 (gp96-Ig/mPTX-35+anti-PD1, boost) post tumor inoculation and monitored for tumor growth inhibition (TGI).

FIG. 46 are representative FACS plots illustrating the percentage of CD8+ OT-I⁺ T cells in the peripheral blood during the peak of CD8 T cell responses on day 4 post-treatment, in the study of FIG. 45.

FIG. 47 is a bar graph illustrating the percentage of CD8+ OT-I⁺ T cells in the peripheral blood during the peak of CD8 T cell responses on day 4 post-treatment, presented as the mean±SEM of the experiment, in the study of FIG. 45.

FIGS. 48A and 48B are tumor growth curves illustrating tumor size, in diameter (mm²), over days 3-19 of the study of FIG. 45. The tumor size was measured every 2 days with a caliper, starting on day 3, for each mouse (n=5) in each of the groups. FIG. 48A illustrates the tumor diameter for vehicle (PBS), mHS-110 (100 ng), mHS-110+1 mg/kg mPTX-35 (mPTX-35med), and mHS-110+anti-PD1. FIG. 48B illustrates the tumor diameter for 1 mg/kg mPTX-35, anti-PD1, 1 mg/kg mPTX-35 (mPTX-35med)+anti-PD1, and mHS-110+1 mg/kg mPTX-35 (mPTX-35med)+anti-PD1.

FIG. 49 are average tumor growth curves, in diameter (mm²), over days 3-19 of the study of FIG. 45. The tumor size was measured every 2 days with a caliper, starting on day 3, for each mouse (n=5) in each of the groups.

FIGS. 50A and 50B are Kaplan-Meier estimator curves illustrating overall, end of study survival, out to day 40 post-tumor inoculation, for each of the groups of the study of FIG. 45. FIG. 50A illustrates Kaplan-Meier curves for vehicle (PBS), mHS-110 (100 ng), mHS-110+1 mg/kg mPTX-35 (mPTX-35med), and mHS-110+anti-PD1. FIG. 50B illustrates Kaplan-Meier curves for 1 mg/kg mPTX-35, anti-PD1, 1 mg/kg mPTX-35 (mPTX-35med)+anti-PD1, and mHS-110+1 mg/kg mPTX-35 (mPTX-35med)+anti-PD1.

FIG. 51 is a non-limiting schematic illustrating a design of the study of effect of a combination of mHS-110 (gp96-Ig), mHS-130 (OX40L-Ig) and mPTX-35 on CD8⁺ T cell expansion kinetics and in therapeutic treatment of established tumors.

FIG. 52 shows anti-tumor CD8+ OT-I T cell expansion, in the peripheral blood with immunization with mHS-110 and mHS-130 with different doses of mPTX-35 (the study of FIG. 51), in the following groups: Vehicle (PBS) (panel A), PTX-35 (1 mg/kg) (panel B), mHS-110+mHS-130 (panel C), mHS-110+mHS-130+mPTX-35 (0.1 mg/kg, “low”) (panel D), mHS-110+mHS-130+mPTX-35 (1 mg/kg, “med”) (panel E), and mHS-110+mHS-130+mPTX-35 (10 mg/kg, “hi”) (panel F). Data is generated using flow cytometry gating, and CD3+CD8+ OT-I+ T cells are shown during the peak expansion on day 4. Total live cells were gated by SSC and FSC parameters, then cells were gated on CD3+ T-cell events, then gated by eGFP+ OT-1+ CD8⁺ T cell events.

FIG. 53 are summary line graphs illustrating anti-tumor CD8+ OT-I T cell expansion over time (total OT-1 cells as a percentage of CD8⁺ T cells), in the peripheral blood with prime and boost immunization of mHS-110 and mHS-130 with different doses of mPTX-35. Graphs show mean±SEM for each group on each day by peripheral blood compartment, in the study of FIG. 51.

FIG. 54 is a bar graph illustrating OT-I cells gated by CD44^{hi} events (% CD8+ OT-I⁺ CD44⁺ T cells) on day 4 in the peripheral blood, exogenous response, in the study of FIG. 51. Statistics shown is a Mann-Whitney, two-tailed test; *p<0.05, **p<0.01, ***p<0.001, ns=p>0.05=not significant.

FIG. 55 are bar graphs illustrating CD8+ KLRG^{hi} IL-7R^{lo} memory cells, exogenous response—day 4, in the study of FIG. 51. SLECs are gated by the Y-axis markers (left graph), and MPECs are gated by the Y-axis markers (right graph). Statistics shown is a Mann-Whitney, two-tailed test; *p<0.05, **p<0.01, ***p<0.001, ns=p>0.05=not significant.

FIG. 56 is a bar graph illustrating CD8+CD44⁺ T cells response in peripheral blood in the study of FIG. 51, with the percent of CD8+CD44⁺ T cells shown over time as mean±SEM.

FIG. 57 is a bar graph illustrating CD8+ KLRG^{hi} IL-7R^{lo} (SLEC) T cells on day 4 of the study of FIG. 51. The statistical test used was Mann-Whitey, two-tailed test; *p<0.05, **p<0.01, ***p<0.001, ns=p>0.05=not significant.

FIG. 58A is a bar graph illustrating OT-I anti-tumor CD8+ T-cell expansion (% of CD8+ OT-I+ T cells) in the spleen on day 21 of the study of FIG. 51. Bars show mean±SEM, statistical analysis performed was non-parametric t-test, Mann-Whitey, two tailed; *p<0.05, **p<0.01, ***p<0.001, ns=p>0.05=not significant.

FIG. 58B is a bar graph illustrating OT-I anti-tumor CD8+ T-cell expansion (% CD8+ OT-I+CD44+CD62L+ T cells) in the spleen on day 21 of the study of FIG. 51. Bars show mean±SEM, statistical analysis performed was non-parametric t-test, Mann-Whitey, two tailed. *p<0.05, **p<0.01, ***p<0.001, ns=p>0.05=not significant.

FIG. 58C is a bar graph illustrating OT-I anti-tumor CD8+ T-cell expansion (% CD8+ OT-I+CD44+CD62L− T cells) in the spleen on day 21 of the study of FIG. 51. Bars show mean±SEM, statistical analysis performed was non-parametric t-test, Mann-Whitey, two tailed. *p<0.05, **p<0.01, ***p<0.001, ns=p>0.05=not significant.

FIG. 59 are average tumor size curves illustrating tumor growth kinetics per group, in the study of FIG. 51. A tumor size (in diameter, mm²), as measured by calipers, is shown over time per treatment.

FIG. 60 are tumor size curves illustrating tumor growth kinetics per mouse per group, in the study of FIG. 51. A tumor size (in diameter, mm²), as measured by calipers, is shown over time per treatment. Graphs show mean±SEM, statistical analysis performed was non-parametric t-test, Mann-Whitey, two tailed; *p<0.05, **p<0.01, ***p<0.001, ns=p>0.05=not significant.

FIG. 61 are a bar graph (left) and a scatter plot (right) illustrating tumor end weights, in grams, for each group, on day 21 of the study of FIG. 51. Graphs show mean±SEM, statistical analysis performed was non-parametric t-test, Mann-Whitey, two tailed; *p<0.05, **p<0.01, ***p<0.001, ns=p>0.05=not significant.

FIG. 62A is a bar graph illustrating tumor-infiltrating leukocytes (TILs) for endogenous T cells (shown as percent of CD3+CD8⁺ T cells), on day 21 of the study of FIG. 51. The graph shows mean±SEM, statistical analysis performed was non-parametric t-test, Mann-Whitey, two tailed; *p<0.05, **p<0.01, ***p<0.001, ns=p>0.05=not significant.

FIG. 62B is a bar graph illustrating TILs for endogenous T cells (shown as percent of CD3+CD8+PD-1+ T cells), on day 21 of the study of FIG. 51. The graph shows mean±SEM, statistical analysis performed was non-parametric t-test, Mann-Whitey, two tailed; *p<0.05, **p<0.01, ***p<0.001, ns=p>0.05=not significant.

FIG. 62C is a bar graph illustrating TILs for endogenous T cells (shown as percent of CD3+CD8+ OT-I+ T cells), on day 21 of the study of FIG. 51. The graph shows mean±SEM, statistical analysis performed was non-parametric t-test, Mann-Whitey, two tailed; *p<0.05, **p<0.01, ***p<0.001, ns=p>0.05=not significant.

FIG. 62D is a bar graph illustrating TILs for endogenous T cells (shown as percent of CD3+CD8+ OT-I+PD-1+ T cells), on day 21 of the study of FIG. 51. The graph shows mean±SEM, statistical analysis performed was non-parametric t-test, Mann-Whitey, two tailed; *p<0.05, **p<0.01, ***p<0.001, ns=p>0.05=not significant.

### DETAILED DESCRIPTION OF THE DISCLOSURE

The present invention is based, in part, on the discovery of surprising immune-modulatory effects of anti-TNFRSF25 agonistic antibodies (e.g. PTX-35). For instance, the present inventors have discovered that anti-TNFRSF25 agonistic antibodies (e.g. PTX-35) cause equal expansion of CD4+ and CD8⁺ T cells. Further, the present inventors have discovered that anti-TNFRSF25 agonistic antibodies (e.g. PTX-35) cause, for both CD4+ and CD8⁺ T cells, a reduction of naïve cells and a transition to memory (e.g. effector and central). Interestingly, the present inventors have discovered that anti-TNFRSF25 agonistic antibodies (e.g. PTX-35) causes an increase in CD4⁺ central memory goes up, but not as much effector, while, for CD8⁺ an increase in effector memory cells is generated, but less so for central memory. Such differential T cell effects of anti-TNFRSF25 agonistic antibodies (e.g. PTX-35) dictate, in various embodiments, determination of therapy effects in a patient and/or selection of additional therapies (e.g. further anti-TNFRSF25 agonistic antibodies (e.g. PTX-35) and/or a combination therapy), The present disclosure provides a method of treating cancer in a patient based, at least in part, on determining a T cell modulatory effect of an effective amount of TNF Receptor Superfamily Member 25 (TNFRSF25) agonistic antibody or antigen binding fragment thereof on a sample from the patient, and administering a second therapy to the patient based on the determined T cell modulatory effect.

The present invention makes use of antibodies targeted to particular epitopes within TNFRSF25. For example, some embodiments make use of PTX-35. The inventors have demonstrated that TNFRSF25 is a potent T cell costimulator due to its specificity for expansion of memory CD4⁺ and CD8⁺ T cells that are known to be potent tumor cell killers. Proliferation of T cells (e.g., human T cells, murine T cells, or macaque T cells) can be stimulated by administering an amount of an anti-TNFRSF25 antibody that is effective to stimulate proliferation of CD4⁺ T cells and/or CD8⁺ T cells. Furthermore, as discussed below, the present disclosure describes differential expansion of central memory and effector memory CD4⁺ T cells and CD8⁺ T cells.

In some aspects, the present disclosure provides a method for treating cancer, comprising administering an effective amount of a TNFRSF25 agonistic antibody or antigen binding fragment thereof, assaying a sample from the patient for a T cell modulatory effect, and administering a second therapy based on the results of the assaying step. In some embodiments, the TNFRSF25 agonistic antibody is PTX-35. PTX-35 is defined by its variable heavy and variable light chain sequences.

T lymphocytes play a central role in regulating immune responses. T cells mature in the thymus, express a T cell receptor (TCR), and can express either CD8 glycoprotein on their surface (CD8⁺ T cells) or CD4 glycoprotein (CD4 cells, helper). Helper T cells express the CD4 surface marker, provide help to B cells for antibody production and help CD8 T cells to develop cytotoxic activity. Other CD4 T cells inhibit antibody production and cytotoxicity. T cells regulate the equilibrium between attack of infected or tumorigenic cells and tolerance to the body's cells. A dysregulated immune attack can lead to autoimmunity, while diminished immune responsiveness results in chronic infection and cancer.

Tumor necrosis factor receptor superfamily member 25 (TNFRSF25) is a TNF-receptor superfamily member that is preferentially expressed by activated and antigen-experienced T lymphocytes. The structural organization of the TNFRSF25 protein is most homologous to TNF receptor 1 (TNFR1). The extracellular domain of TNFRSF25 includes four cysteine-rich domains, and the cytoplasmic region contains a death domain known to signal apoptosis. Alternative splicing of the TNFRSF25 gene in B and T cells encounters a program change upon T cell activation, which predominantly produces full-length, membrane bound isoforms, and is involved in controlling lymphocyte proliferation induced by T cell activation. TNFRSF25 is activated by its ligand, TNF-like protein 1A (TL1A), also referred to as TNFSF15, which is rapidly upregulated in antigen presenting cells and in some endothelial cells following Toll-Like Receptor or Fc receptor activation. TL1A has co-stimulatory activity for TNFRSF25-expressing T cells through the activation of NF-κB and suppression of apoptosis by up-regulation of c-IAP2. TNFRSF25 signaling increases the sensitivity of T cells to endogenous IL-2, and enhances T cell proliferation.

The T cell modulatory effect in accordance with embodiments of the present disclosure can be exhibited in various ways. For example in some embodiments, the T cell modulatory effect is an expansion of CD4⁺ T cells and CD8⁺ T cells. In some embodiments, the T cell modulatory effect is equal (or substantially equal, such as within 15% of one another) expansion of CD4+ and CD8⁺ T cells. For example, the expansion can provide a ratio of CD4⁺ T cells to CD8⁺ T cells of about 1:1. In some embodiments, the expansion is differential and it provides a ratio of CD4⁺ T cells to CD8⁺ T cells of about 1.5:1, or about 2:1, or about 1:1.5, or about 1:2.

In some embodiments, the expansion of CD4⁺ T cells and CD8⁺ T cells can be at least about 5%, or at least about 6%, or at least about 7%, or at least about 8%, or at least about 9%, or at least about 10%, or at least about 11%, or at least about 12%, or at least about 13%, or at least about 14%, or at least about 15%, or at least about 16%, or at least about 17%, or at least about 18%, or at least about 19%, or at least about 20%, or at least about 30%, or at least about 40%, or at least about 50%, or at least about 60%, or at least about 70%, or at least about 80%, or at least about 90% increase in the number of CD4+ T cells and CD8+ T cells, as compared to the number of CD4+ T cells and CD8+ T cells in the subject prior to administration of the composition, or as compared to a number of CD4+ T cells and CD8+ T cells in a control subject or population of subjects to whom the composition was not administered.

In some embodiments, the expansion comprises an expansion sufficient to treat cancer in a patient, for example, sufficient to lead to an increase in a number of tumor-infiltrating lymphocytes in the patient, and/or a decrease in at least one of a tumor size and a tumor growth rate in the patient. In some embodiments, the expansion comprises an expansion sufficient to reduce a tumor size in a subject (e.g., a patient) by at least about 5%, or by at least about 10%, or by at least about 15%, or by at least about 20%, or by at least about 25%, or by at least about 30%, or by at least about 40%, or by at least about 50% as compared to a tumor size in the patient prior to administration of the composition, or as compared to a tumor size in a control subject or population of subjects to whom the composition was not administered.

In some embodiments, the expansion of CD4⁺ T cells and CD8⁺ T cells comprises an expansion sufficient to reduce the progression rate of cancer in a subject administered a composition comprising an effective amount of a TNFRSF25 agonistic antibody or antigen binding fragment thereof in accordance with embodiments of the present disclosure (e.g., by at least about 10 percent, about 20 percent, about 25 percent, about 50 percent, about 60 percent, about 70 percent, about 75 percent, about 80 percent, about 90 percent, or more than 90 percent), as compared to the rate of cancer progression in the subject prior to administration of the composition, or as compared to the rate of cancer progression in a control subject or population of subjects to whom the composition was not administered.

In some embodiments, the expansion CD4⁺ T cells and CD8⁺ T cells in a subject administered a composition in accordance with embodiments of the present disclosure is at least about 0.1-fold expansion, or at least about 0.5-fold expansion, or at least about 1-fold expansion, or at least about 1.5-fold expansion, or at least about 2-fold expansion, or at least about 3-fold expansion, or at least about 4-fold expansion, or at least about 5-fold expansion. In some embodiments, the expansion is at least about 10-fold expansion, or at least about 20-fold expansion, or at least about 30-fold expansion, or at least about 40-fold expansion, or at least about 50-fold expansion, or at least about 60-fold expansion, or at least about 70-fold expansion, or at least about 80-fold expansion, or at least about 90-fold expansion, or at least about 100-fold expansion, as compared to expansion in the subject prior to administration of the composition, or as compared to expansion in a control subject or population of subjects to whom the composition was not administered. In some embodiments, the expansion is greater than an about 100-fold expansion.

In some embodiments, the T cell modulatory effect is a differential expansion of effector memory and central memory T cells. It is generally known that, upon stimulation or priming by antigen (Ag), T cells proliferate and differentiate into effector and memory T cells. For example, central memory cells can be located in the secondary lymphoid organs, while effector memory cells can be located in the recently infected tissues. Thus, memory T cells in the circulation can be central memory (CM) and effector (EM) memory T cells.

In some embodiments, the differential expansion of effector memory and central memory T cells provides a ratio of effector memory T cells to central memory T cells in the range of at or about 3:1 to about 1:1.1. For example, the expansion may provide a ratio of effector memory T cells to central memory T cells of at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, or 3:1. In some embodiments, the differential expansion of effector memory and central memory T cells is a differential expansion such that the expansion provides a ratio of central memory T cells to effector memory T cells of at or about 3:1 to about 1:1.1. For example, the expansion may provide a ratio of central memory T cells to effector memory T cells of at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, or 3:1.

In some embodiments, the inventors have discovered, for both CD4⁺ and CD8⁺ T cells, that expression of naïve cells decreases whereas expression of memory cells increases. In some embodiments, the differential expansion of effector memory and central memory T cells is differential expansion of CD4⁺ central memory T cells to CD4⁺ effector memory T cells. Thus, the inventors have discovered that differential expansion of effector memory and central memory T cells provides an increase in a ratio of CD4⁺ central memory T cells to CD4⁺ effector memory T cells. In other words, the CD4⁺ central memory T cells expand to a larger degree than CD4⁺ effector memory T cells.

In some embodiments, differential expansion of effector memory and central memory T cells is differential expansion of CD8⁺ central memory T cells to CD8⁺ effector memory T cells. Thus, the inventors have discovered that the differential expansion provides an increase in a ratio of CD8⁺ effector memory to CD8⁺ central memory T cells.

The second therapy administered based on results of the assaying the sample from the patient for a T cell modulatory effect can include one or more of various types of therapy.

In some embodiments, the second therapy is a checkpoint inhibitor. The checkpoint inhibitor can be an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2.

In some embodiments, the checkpoint inhibitor is an agent that targets PD-1, and such agent is an antibody or antibody format specific for PD-1, optionally selected from nivolumab, pembrolizumab, and pidilizumab. In some embodiments, the checkpoint inhibitor is an agent that targets PD-L1 and it is an antibody or antibody format specific for PD-L1, optionally selected from atezolizumab, avelumab, durvalumab, and BMS-936559.

In some embodiments, the agent that targets CTLA-4 is an antibody or antibody format specific for CTLA-4, optionally selected from ipilimumab and tremelimumab.

In some embodiments, the present disclosure provides a method for treating cancer that comprises administering an effective amount of a TNFRSF25 agonistic antibody or antigen binding fragment thereof to a patient in need thereof, administering a biological adjuvant to the patient, and administering a checkpoint inhibitor molecule to the patient. In some embodiments, the method results in an increase in an antigen-specific CD8 T cell response in the patient, increase in a number of tumor-infiltrating lymphocytes in the patient, and/or a decrease in at least one of a tumor size and a tumor growth rate in the patient.

In some embodiments, the present disclosure provides a method for treating cancer that comprises administering an effective amount of a TNFRSF25 agonistic antibody or antigen binding fragment thereof to a patient in need thereof and administering a biological adjuvant to the patient. The patient may be undergoing treatment with a checkpoint inhibitor molecule. In some embodiments, the method results in an increase in a number of antigen-specific CD8 T cells in the patient, an increase in a number of tumor-infiltrating lymphocytes in the patient, and/or a decrease in at least one of a tumor size and a tumor growth rate in the patient.

In some embodiments, the checkpoint inhibitor used in the methods for treating cancer in accordance with embodiments of the present disclosure can be an agent that targets PD-1, and such agent may be an antibody or antibody format specific for PD-1, optionally selected from nivolumab, pembrolizumab, and pidilizumab. In some embodiments, the checkpoint inhibitor is an agent that targets PD-L1 and it is an antibody or antibody format specific for PD-L1, optionally selected from atezolizumab, avelumab, durvalumab, and BMS-936559.

In some embodiments, the biological adjuvant comprises a secretable vaccine protein such as, for example, gp96. In some embodiments, the secretable vaccine protein is a gp96-Ig fusion protein, e.g. the gp96-Ig fusion protein that lacks the gp96 KDEL (SEQ ID NO: 10) sequence. In some embodiments, the biological adjuvant further comprises a T cell costimulatory fusion protein which enhances activation of antigen-specific T cells. The T cell costimulatory fusion protein can be selected from OX40L-Ig, or a portion thereof that binds to OX40, ICOSL-Ig, or a portion thereof that binds to ICOS, 4 1BBL-Ig, or a portion thereof that binds to 4-1BBR, TL1A-Ig, or a portion thereof that binds to TNFRSF25, GITRL-Ig, or a portion thereof that binds to GITR, CD40L-Ig, or a portion thereof that binds to CD40, and, CD70-Ig, or a portion thereof that binds to CD27. The T cell costimulatory fusion protein can be an Ig fusion protein. For example, in some embodiments, the T cell costimulatory fusion protein is OX40L-Ig administered in combination with the gp96-Ig fusion protein lacking the gp96 KDEL (SEQ ID NO: 10) sequence.

The present disclosure makes use of TNFRSF25 agonistic antibodies or antigen binding fragment thereof. In various embodiments, the antibody is an antibody (e.g., human, hamster, feline, mouse, cartilaginous fish, or camelid antibodies), and any derivative or conjugate thereof, that specifically binds to TNFRSF25. Non-limiting examples of antibodies include monoclonal antibodies, polyclonal antibodies, humanized antibodies, multi-specific antibodies (e.g., bi-specific antibodies), single-chain antibodies (e.g., single-domain antibodies, camelid antibodies, and cartilaginous fish antibodies), chimeric antibodies, feline antibodies, and felinized antibodies. Monoclonal antibodies are homogeneous populations of antibodies to a particular epitope of an antigen. Polyclonal antibodies are heterogeneous populations of antibody molecules that are contained in the sera of the immunized animals.

An isolated polypeptide can yield a single major band on a non-reducing polyacrylamide gel. An isolated polypeptide can be at least about 75% pure (e.g., at least 80%, 85%, 90%, 95%, 97%, 98%, 99%, or 100% pure). Isolated polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

In some embodiments, an antigen binding fragment that specifically binds to TNFRSF25 is provided. Such antigen binding fragment, in embodiments, is any portion of a full-length antibody that contains at least one variable domain (e.g., a variable domain of a mammalian (e.g., feline, human, hamster, or mouse) heavy or light chain immunoglobulin, a camelid variable antigen binding domain (VHH), or a cartilaginous fish immunoglobulin new antigen receptor (Ig-NAR) domain) that is capable of specifically binding to an antigen. Non-limiting examples of antibody fragments include Fab, Fab′, F(ab′)2, and Fv fragments, diabodies, linear antibodies, and multi-specific antibodies formed from antibody fragments. Additional antibody fragments containing at least one camelid VHH domain or at least one cartilaginous fish Ig-NAR domain include mini-bodies, micro-antibodies, subnano-antibodies, and nano-antibodies, and any of the other forms of antibodies described, for example, in U.S. Publication No. 2010/0092470.

An antibody can be of the IgA-, IgD-, IgE, IgG- or IgM-type, including IgG- or IgM-types such as, without limitation, IgG1-, IgG2-, IgG3-, IgG4-, IgM1- and IgM2-types. For example, in some cases, the antibody is of the IgG1-, IgG2- or IgG4-type.

In some embodiments, antibodies as provided herein can be fully human or humanized antibodies. In embodiments, the human antibody is an antibody that is encoded by a nucleic acid (e.g., a rearranged human immunoglobulin heavy or light chain locus) present in the genome of a human. In some embodiments, a human antibody can be produced in a human cell culture (e.g., feline hybridoma cells). In some embodiments, a human antibody can be produced in a non-human cell (e.g., a mouse or hamster cell line). In some embodiments, a human antibody can be produced in a bacterial or yeast cell.

Human antibodies can avoid certain problems associated with xenogeneic antibodies, such as antibodies that possess murine or rat variable and/or constant regions. For example, because the effector portion is human, it can interact better with other parts of the human immune system, e.g., to destroy target cells more efficiently by complement-dependent cytotoxicity or antibody-dependent cellular cytotoxicity. In addition, the human immune system should not recognize the antibody as foreign. Further, half-life in human circulation will be similar to naturally occurring human antibodies, allowing smaller and less frequent doses to be given. Methods for preparing human antibodies are known in the art.

In some embodiments, the antibody is a humanized antibody, e.g., that contains minimal sequence derived from non-human (e.g., mouse, hamster, rat, rabbit, or goat) immunoglobulin. Humanized antibodies generally are chimeric or mutant monoclonal antibodies from mouse, rat, hamster, rabbit or other species, bearing human constant and/or variable region domains or specific changes. In non-limiting examples, humanized antibodies are human antibodies (recipient antibody) in which hypervariable region (HVR) residues of the recipient antibody are replaced by HVR residues from a non-human species (donor) antibody, such as a mouse, rat, rabbit, or goat antibody having the desired specificity, affinity, and capacity. In some embodiments, Fv framework residues of the human immunoglobulin can be replaced by corresponding non-human residues. In some embodiments, humanized antibodies can contain residues that are not found in the recipient antibody or in the donor antibody. Such modifications can be made to refine antibody performance, for example.

In some embodiments, a humanized antibody can contain substantially all of at least one, and typically two, variable domains, in which all or substantially all of the hypervariable loops (CDRs) correspond to those of a non-human immunoglobulin, while all or substantially all of the framework regions are those of a human immunoglobulin sequence. A humanized antibody also can contain at least a portion of an immunoglobulin constant (Fc) region, typically that of a human immunoglobulin.

In some embodiments, a humanized antibody or antigen binding fragment as provided herein can have reduced or minimal effector function (e.g., as compared to corresponding non-humanized antibody), such that it does not stimulate effector cell action to the same extent that a corresponding non-humanized antibody would.

Techniques for generating humanized antibodies are well known to those of skill in the art. In some embodiments, controlled rearrangement of antibody domains joined through protein disulfide bonds to form new, artificial protein molecules or “chimeric” antibodies can be utilized (Konieczny et al., Haematologia (Budap.) 14:95, 1981). Recombinant DNA technology can be used to construct gene fusions between DNA sequences encoding mouse antibody variable light and heavy chain domains and human antibody light and heavy chain constant domains (Morrison et al., Proc Natl Acad Sci USA 81:6851, 1984). For example, DNA sequences encoding antigen binding portions or CDRs of murine monoclonal antibodies can be grafted by molecular means into DNA sequences encoding frameworks of human antibody heavy and light chains (Jones et al., Nature 321:522, 1986; and Riechmann et al., Nature 332:323, 1988). Expressed recombinant products are called “reshaped” or humanized antibodies, and contain the framework of a human antibody light or heavy chain and antigen recognition portions, CDRs, of a murine monoclonal antibody.

Other methods for designing heavy and light chains and for producing humanized antibodies are described in, for example, U.S. Pat. Nos. 5,530,101; 5,565,332; 5,585,089; 5,639,641; 5,693,761; 5,693,762; and 5,733,743. Yet additional methods for humanizing antibodies are described in U.S. Pat. Nos. 4,816,567; 4,935,496; 5,502,167; 5,558,864; 5,693,493; 5,698,417; 5,705,154; 5,750,078; and 5,770,403, for example.

In embodiments, the antibody is a single-chain antibody, e.g. a single polypeptide that contains at least one variable binding domain (e.g., a variable domain of a mammalian heavy or light chain immunoglobulin, a camelid VHH, or a cartilaginous fish (e.g., shark) Ig-NAR domain) that is capable of specifically binding to an antigen. Non-limiting examples of single-chain antibodies include single-domain antibodies.

In embodiments, the antibody is a single-domain antibody, e.g., a polypeptide that contains one camelid VHH or at least one cartilaginous fish Ig-NAR domain that is capable of specifically binding to an antigen. Non-limiting examples of single-domain antibodies are described, for example, in U.S. Publication No. 2010/0092470.

In embodiments, the antibody specifically binds to a particular antigen, e.g., TNFRSF25, when it binds to that antigen in a sample, and does not recognize and bind, or recognizes and binds to a lesser extent, other molecules in the sample. In some embodiments, an antibody or an antigen binding fragment thereof can selectively bind to an epitope with an affinity (Kd) equal to or less than, for example, about 1×10⁻⁶ M (e.g., equal to or less than about 1×10⁻⁹ M, equal to or less than about 1×10⁻¹⁰ M, equal to or less than about 1×10⁻¹¹ M, or equal to or less than about 1×10⁻¹² M) in phosphate buffered saline. The ability of an antibody or antigen binding fragment to specifically bind a protein epitope can be determined using any of the methods known in the art or those methods described herein (e.g., by Biacore/Surface Plasmon Resonance). This can include, for example, binding to TNFRSF25 on live cells as a method to stimulate caspase activation in live transformed cells, binding to an immobilized target substrate including human TNFRSF25 fusion proteins as detected using an ELISA method, binding to TNFRSF25 on live cells as detected by flow cytometry, or binding to an immobilized substrate by surface plasmon resonance (including ProteOn).

In some embodiments, the TNFRSF25 agonistic antibody or antigen binding fragment thereof comprises (i) a heavy chain variable region comprising heavy chain CDR1, CDR2, and CDR3 sequences, wherein the heavy chain CDR1 sequence is GFTFSNHDLN (SEQ ID NO: 1) or a variant thereof, the heavy chain CDR2 sequence is YISSASGLISYADAVRG (SEQ ID NO: 2) or a variant thereof; and the heavy chain CDR3 sequence is DPAYTGLYALDF (SEQ ID NO: 3) or a variant thereof or DPPYSGLYALDF (SEQ ID NO: 4) or a variant thereof; and (ii) a light chain variable region comprising light chain CDR1, CDR2, and CDR3 sequences, wherein the light chain CDR1 sequence is TLSSELSWYTIV (SEQ ID NO: 5) or a variant thereof, the light chain CDR2 sequence is LKSDGSHSKGD (SEQ ID NO: 6) or a variant thereof, and the light chain CDR3 sequence is CGAGYTLAGQYGWV (SEQ ID NO: 7) or a variant thereof. In various embodiments, the variant TNFRSF25 agonistic antibody or antigen binding fragment thereof comprises an amino acid sequence having one or more amino acid mutations (e.g., substitutions or deletions) relative to any of the sequences disclosed herein, e.g. the CDRs (e.g. any of heavy chain CDR1, CDR2, or CDR3 and any of light chain CDR1, CDR2, or CDR3). In some embodiments, the one or more amino acid mutations may be independently selected from substitutions, insertions, deletions, and truncations. In embodiments, the TNFRSF25 agonistic antibody or antigen binding fragment thereof, e.g. the CDRs (e.g. any of heavy chain CDR1, CDR2, or CDR3 and any of light chain CDR1, CDR2, or CDR3), comprises a sequence that has about 1, 2, 3, 4, 5, 6, 7, 8, 9, or 10 or more amino acid mutations with respect to any one of the amino acid sequences disclosed herein (e.g. any of SEQ ID Nos: 1-7).

In some embodiments, the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4). In these embodiments, the variable region FW sequences in the heavy chain variable region are heavy chain variable region FW sequences, and the variable region FW sequences in the light chain variable region are light chain variable region FW sequences. In some embodiments, the variable region FW sequences are human.

In some embodiments, the TNFRSF25 agonistic antibody or antigen binding fragment thereof further comprises human heavy chain and light chain constant regions. The constant regions can be selected from the group consisting of human IgG1, IgG2, IgG3, and IgG4. For example, in an embodiment, the constant regions are IgG1. In another embodiment, the constant regions are IgG4.

Antibodies having specific binding affinity for TNFRSF25 can be produced using standard methods. For example, a TNFRSF25 polypeptide can be recombinantly produced, purified from a biological sample (e.g., a heterologous expression system), or chemically synthesized, and used to immunize host animals, including rabbits, chickens, mice, guinea pigs, or rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin and dinitrophenol. Monoclonal antibodies can be prepared using a TNFRSF25 polypeptide and standard hybridoma technology. In particular, monoclonal antibodies can be obtained by any technique that provides for the production of antibody molecules by continuous cell lines in culture such as described by Kohler et al. (Nature 256:495, 1975), the human B-cell hybridoma technique of Kosbor et al. (Immunology Today, 4:72, 1983) or Cote et al. (Proc. Natl. Acad. Sci. USA, 80:2026, 1983), and the EBV-hybridoma technique described by Cole et al. (Monoclonal Antibodies and Cancer Therapy, Alan R. Liss, Inc., pp. 77-96, 1983). Such antibodies can be of any immunoglobulin class including IgG, IgM, IgE, IgA, IgD, and any subclass thereof. The hybridoma producing the monoclonal antibodies can be cultivated in vitro and in vivo.

In some embodiments, the TNFRSF25 agonistic antibody or antigen binding fragment thereof comprises a heavy chain variable region of the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of about 85% to about 99% identity thereto. In some embodiments, the TNFRSF25 agonistic antibody or antigen binding fragment thereof comprises a heavy chain variable region of the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of about 85%, or about 86%, or about 87%, or about 88%, or about 89%, or about 90%, or about 91%, or about 92%, or about 93%, or about 94%, or about 95%, or about 96%, or about 97%, or about 98%, or about 99% identity thereto.

In some embodiments, the TNFRSF25 agonistic antibody or antigen binding fragment thereof comprises a light chain variable region of the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of about 85 to % about 99% identity thereto. In some embodiments, the TNFRSF25 agonistic antibody or antigen binding fragment thereof comprises a light chain variable region of the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of about 85%, or about 86%, or about 87%, or about 88%, or about 89%, or about 90%, or about 91%, or about 92%, or about 93%, or about 94%, or about 95%, or about 96%, or about 97%, or about 98%, or about 99% identity thereto.

In some embodiments, “PTX-35” as used in embodiments of the present invention has the heavy chain variable region of the amino acid sequence of SEQ ID NO: 8 and the light chain variable region of the amino acid sequence of SEQ ID NO: 9. In some embodiments, PTX-35 has the heavy chain variable region of the amino acid sequence of SEQ ID NO: 8 or an amino acid sequence of about 85% to about 99% identity thereto, and the light chain variable region of the amino acid sequence of SEQ ID NO: 9 or an amino acid sequence of about 85% to about 99% identity thereto.

In some embodiments, amino acid substitutions can be made by selecting conservative substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of conservative substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. In some embodiments, an amino acid substitution can be non-conservative, such that a member of one of the amino acid classes described above is exchanged for a member of another class.

In various embodiments, there are provided methods of treating cancer with the present TNFRSF25 agonistic antibody or antigen binding fragment thereof (e.g. PTX-35) involving a second therapy, e.g., combination therapy, e.g., based on the patient screening methods described herein. In some embodiments, the second therapy is further administration of the present TNFRSF25 agonistic antibody or antigen binding fragment thereof (e.g. PTX-35). In some embodiments, the second therapy is radiation therapy which can be administered before, after, or at least partially simultaneously with the present TNFRSF25 agonistic antibody or antigen binding fragment thereof (e.g. PTX-35). In some embodiments, the second therapy is one or more checkpoint inhibitors which can be administered before, after, or at least partially simultaneously with the present TNFRSF25 agonistic antibody or antigen binding fragment thereof (e.g. PTX-35). In some embodiments, the methods of treatment can further include the use of photodynamic therapy. Furthermore, in some embodiments, the second therapy is chemotherapy, which can be administered before, after, or at least partially simultaneously with the present TNFRSF25 agonistic antibody or antigen binding fragment thereof (e.g. PTX-35).

In some embodiments, the second therapy is further administration of the TNFRSF25 agonistic antibody or antigen binding fragment thereof. Thus, one or more additional doses of the TNFRSF25 agonistic antibody or antigen binding fragment thereof can be administered to the patient. In some embodiments, the additional dose(s) of TNFRSF25 agonistic antibody or antigen binding fragment thereof can be administered in combination with radiation therapy and/or a checkpoint inhibitor.

In some embodiments, the second therapy is a biological adjuvant. The biological adjuvant may comprise a secretable vaccine protein, which, in some embodiments, can be gp96. In some embodiments, the secretable vaccine protein is a gp96-Ig fusion protein. In some embodiments, the gp96-Ig fusion protein may optionally lack the gp96 KDEL (SEQ ID NO: 10) sequence, and in such embodiments the gp96-Ig fusion protein may be referred to herein as ImPACT, see, e.g., U.S. Pat. No. 8,685,384, the entirety contents of which are incorporated by reference. As used herein, ImPACT is Viagenpumatucel-L (also referred to herein as “HS-110”), which is a proprietary, allogeneic tumor cell vaccine expressing a recombinant secretory form of the gp96 fusion (gp96-Ig), with potential antineoplastic activity. The heat shock protein gp96 serves as a chaperone for peptides on their way to MHC class I molecules expressed on antigen-presenting or dendritic cell. Gp96 obtained from tumor cells and used as a vaccine can induce specific tumor immunity, presumably through the transport of tumor-specific peptides to antigen-presenting cells (APCs) (J Immunol 1999, 163(10):5178-5182). For example, gp96-associated peptides are cross-presented to CD8 cells by dendritic cells (DCs) upon uptake of the scavenger receptor. Upon administration of HS-110, irradiated live tumor cells continuously secrete gp96-Ig along with its chaperoned tumor associated antigens (TAAs) into the dermal layers of the skin, thereby activating antigen presenting cells, natural killer cells and priming potent cytotoxic T lymphocytes (CTLs) to respond against TAAs presented on the endogenous tumor cells. Furthermore, HS-110 induces long-lived memory T cells that can fight recurring cancer cells.

In some embodiments, the Ig tag in the gp96-Ig fusion protein comprises the Fc region of human IgG1, IgG2, IgG3, IgG4, IgM, IgA, or IgE.

In some embodiments, the biological adjuvant further comprises a T cell costimulatory fusion protein which enhances activation of antigen-specific T cells and which can be selected from OX40L-Ig, or a portion thereof that binds to OX40, ICOSL-Ig, or a portion thereof that binds to ICOS, 4 1BBL-Ig, or a portion thereof that binds to 4-1BBR, TL1A-Ig, or a portion thereof that binds to TNFRSF25, GITRL-Ig, or a portion thereof that binds to GITR, CD40L-Ig, or a portion thereof that binds to CD40, and, CD70-Ig, or a portion thereof that binds to CD27. The T cell costimulatory fusion protein can be administered in combination with gp96-Ig vaccination. In some embodiments, the biological adjuvant is ComPACT, see, e.g., U.S. Pat. No. 10,046,047, the entire content of which is hereby incorporated herein by reference.

In some embodiments, the T cell costimulatory fusion protein is OX40L-Ig (HS-130) administered in combination with the gp96-Ig fusion protein lacking the gp96 KDEL (SEQ ID NO: 10) sequence (i.e., HS-110). Synergy of PTX-35 with gp96-Ig (HS-110) and OX40L-Ig (HS-130) in prevention of the outgrowth of tumor and expansion of antigen-specific CD8⁺ T cells was demonstrated. In some embodiments, gp96-Ig and OX40L-Ig are secreted by separate lines (mHS-110 and mHS-130, respectively). In such embodiments, the present TNFRSF25 agonistic antibody or antigen binding fragment thereof (e.g., PTX-35) can be co-administered with one or both of mHS-110 and mHS-130. In other embodiments, a single cell line secrets both gp96-Ig and OX40L-Ig (e.g., ComPACT), and PTX-35 can be administered along with the cell secreting both gp96-Ig and OX40L-Ig (or another T cell costimulatory fusion protein). In some embodiments, the T cell costimulatory fusion protein is an Ig fusion protein. The Ig tag in the T cell costimulatory fusion protein may optionally comprises the Fc region of human IgG1, IgG2, IgG3, IgG4, IgM, IgA, or IgE.

The secretable vaccine protein and/or T cell costimulatory fusion protein can be encoded on an expression vector. The expression vector can thus include a first nucleotide sequence that encodes the secretable vaccine protein (e.g., a gp96-Ig fusion protein), and a second nucleotide sequence that encodes the T cell costimulatory fusion protein. The T cell costimulatory fusion protein enhances activation of antigen-specific T cells when administered to a subject. The expression vector can be incorporated into a human tumor cell.

In some embodiments, as mentioned above, a gp96-Ig fusion protein is constructed by replacing the KDEL retention sequence of gp96, normally an endoplasmatic reticulum-resident chaperone peptide, with the Fc portion of human IgG1, using an optional linker. The Fc portion of human IgG1 can include the CH2-CH3 domains and can optionally include the hinge region at the N-terminus (hinge-CH2-CH3). In some cases, the IgG1 hinge serves as the linker joining the gp96 protein and the Fc domain.

In some embodiments, the vector comprising the gp96-Ig fusion protein comprises a linker. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In some embodiments, the gp96-Ig fusion protein can be expressed in DNA- or RNA-based vectors including nucleotide sequences that encode the secretable vaccine protein. Nucleic acid sequences encoding a vaccine protein fusion protein (e.g., a gp96-Ig fusion protein) can be constructed and cloned into an expression vector. The expression vector can be introduced into host cells, either of which can be administered to a subject to treat cancer. For example, the gp96-Ig based vaccines can be generated to stimulate antigen specific immune responses against tumor antigens.

In some embodiments, cDNA or DNA sequences encoding the vaccine protein fusion (e.g., a gp96-Ig fusion) can be obtained (and, if desired, modified) using conventional DNA cloning and mutagenesis methods, DNA amplification methods, and/or synthetic methods. In general, a sequence encoding a vaccine protein fusion protein (e.g., a gp96-Ig fusion protein) can be inserted into a cloning vector for genetic modification and replication purposes prior to expression. Each coding sequence can be operably linked to a regulatory element, such as a promoter, for purposes of expressing the encoded protein in suitable host cells in vitro and in vivo.

Both prokaryotic and eukaryotic vectors can be used for expression of the vaccine protein (e.g., gp96-Ig) in the methods provided herein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and APL. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful.

A variety of regulatory regions can be used for expression of the vaccine protein (e.g., gp96-Ig) and T cell costimulatory fusions in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the fusion proteins in recombinant host cells.

In one aspect, the present disclosure contemplates the use of inducible promoters capable of effecting high level of expression transiently in response to a cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Animal regulatory regions that exhibit tissue specificity and have been utilized in transgenic animals also can be used in tumor cells of a particular tissue type: the elastase I gene control region that is active in pancreatic acinar cells (Swift et al., Cell 1984, 38:639-646; Ornitz et al., Cold Spring Harbor Symp Quant Biol 1986, 50:399-409; and MacDonald, Hepatology 1987, 7:425-515); the insulin gene control region that is active in pancreatic beta cells (Hanahan, Nature 1985, 315:115-122), the immunoglobulin gene control region that is active in lymphoid cells (Grosschedl et al., Cell 1984, 38:647-658; Adames et al., Nature 1985, 318:533-538; and Alexander et al., Mol Cell Biol 1987, 7:1436-1444), the mouse mammary tumor virus control region that is active in testicular, breast, lymphoid and mast cells (Leder et al., Cell 1986, 45:485-495), the albumin gene control region that is active in liver (Pinkert et al., Genes Devel, 1987, 1:268-276), the alpha-fetoprotein gene control region that is active in liver (Krumlauf et al., Mol Cell Biol 1985, 5:1639-1648; and Hammer et al., Science 1987, 235:53-58); the alpha 1-antitrypsin gene control region that is active in liver (Kelsey et al., Genes Devel 1987, 1:161-171), the beta-globin gene control region that is active in myeloid cells (Mogram et al., Nature 1985, 315:338-340; and Kollias et al., Cell 1986, 46:89-94); the myelin basic protein gene control region that is active in oligodendrocyte cells in the brain (Readhead et al., Cell 1987, 48:703-712); the myosin light chain-2 gene control region that is active in skeletal muscle (Sani, Nature 1985, 314:283-286), and the gonadotropic releasing hormone gene control region that is active in the hypothalamus (Mason et al., Science 1986, 234:1372-1378).

An expression vector also can include transcription enhancer elements, such as those found in SV40 virus, Hepatitis B virus, cytomegalovirus, immunoglobulin genes, metallothionein, and β-actin (see, Bittner et al., Meth. Enzymol 1987, 153:516-544; and Gorman, Curr Op Biotechnol 1990, 1:36-47). In addition, an expression vector can contain sequences that permit maintenance and replication of the vector in more than one type of host cell, or integration of the vector into the host chromosome. Such sequences include, without limitation, to replication origins, autonomously replicating sequences (ARS), centromere DNA, and telomere DNA.

In addition, an expression vector can contain one or more selectable or screenable marker genes for initially isolating, identifying, or tracking host cells that contain DNA encoding fusion proteins as described herein. For long term, high yield production of gp96-Ig and T cell costimulatory fusion proteins, stable expression in mammalian cells can be useful. A number of selection systems can be used for mammalian cells. For example, the Herpes simplex virus thymidine kinase (Wigler et al., Cell 1977, 11:223), hypoxanthine-guanine phosphoribosyltransferase (Szybalski and Szybalski, Proc Natl Acad Sci USA 1962, 48:2026), and adenine phosphoribosyltransferase (Lowy et al., Cell 1980, 22:817) genes can be employed in tk-, hgprt-, or aprt-cells, respectively. In addition, antimetabolite resistance can be used as the basis of selection for dihydrofolate reductase (dhfr), which confers resistance to methotrexate (Wigler et al., Proc Natl Acad Sci USA 1980, 77:3567; O'Hare et al., Proc Natl Acad Sci USA 1981, 78:1527); gpt, which confers resistance to mycophenolic acid (Mulligan and Berg, Proc Natl Acad Sci USA 1981, 78:2072); neomycin phosphotransferase (neo), which confers resistance to the aminoglycoside G-418 (Colberre-Garapin et al., J Mol Biol 1981, 150:1); and hygromycin phosphotransferase (hyg), which confers resistance to hygromycin (Santerre et al., Gene 1984, 30:147). Other selectable markers, such as, e.g., histidinol and Zeocin™, also can be used.

In some embodiments, viral-based expression systems also can be used with mammalian cells to produce gp96-Ig. Vectors using DNA virus backbones have been derived from simian virus 40 (SV40) (Hamer et al., Cell 1979, 17:725), adenovirus (Van Doren et al., Mol Cell Biol 1984, 4:1653), adeno-associated virus (McLaughlin et al., J Virol 1988, 62:1963), and bovine papillomas virus (Zinn et al., Proc Natl Acad Sci USA 1982, 79:4897). When an adenovirus is used as an expression vector, the donor DNA sequence may be ligated to an adenovirus transcription/translation control complex, e.g., the late promoter and tripartite leader sequence.

This fusion gene may then be inserted in the adenovirus genome by in vitro or in vivo recombination. Insertion in a non-essential region of the viral genome (e.g., region E1 or E3) can result in a recombinant virus that is viable and capable of expressing heterologous products in infected hosts. (See, e.g., Logan and Shenk, Proc Natl Acad Sci USA 1984, 81:3655-3659).

Bovine papillomavirus (BPV) can infect many higher vertebrates, including man, and its DNA replicates as an episome. A number of shuttle vectors have been developed for recombinant gene expression which exist as stable, multicopy (20-300 copies/cell) extrachromosomal elements in mammalian cells. Typically, these vectors contain a segment of BPV DNA (the entire genome or a 69% transforming fragment), a promoter with a broad host range, a polyadenylation signal, splice signals, a selectable marker, and “poisonless” plasmid sequences that allow the vector to be propagated in E. coli. Following construction and amplification in bacteria, the expression gene constructs are transfected into cultured mammalian cells by, for example, calcium phosphate coprecipitation. For those host cells that do not manifest a transformed phenotype, selection of transformants is achieved by use of a dominant selectable marker, such as histidinol and G418 resistance.

Alternatively, the vaccinia 7.5K promoter can be used. (See, e.g., Mackett et al., Proc Natl Acad Sci USA 1982, 79:7415-7419; Mackett et al., J Virol 1984, 49:857-864; and Panicali et al., Proc Natl Acad Sci USA 1982, 79:4927-4931.) In cases where a human host cell is used, vectors based on the Epstein-Barr virus (EBV) origin (OriP) and EBV nuclear antigen 1 (EBNA-1; a trans-acting replication factor) can be used. Such vectors can be used with a broad range of human host cells, e.g., EBO-pCD (Spickofsky et al., DNA Prot Eng Tech 1990, 2:14-18); pDR2 and λDR2 (available from Clontech Laboratories).

Gp96-Ig fusion proteins also can be made with retrovirus-based expression systems. Retroviruses, such as Moloney murine leukemia virus, can be used since most of the viral gene sequence can be removed and replaced with exogenous coding sequence while the missing viral functions can be supplied in trans. In contrast to transfection, retroviruses can efficiently infect and transfer genes to a wide range of cell types including, for example, primary hematopoietic cells. Moreover, the host range for infection by a retroviral vector can be manipulated by the choice of envelope used for vector packaging.

For example, a retroviral vector can comprise a 5′ long terminal repeat (LTR), a 3′ LTR, a packaging signal, a bacterial origin of replication, and a selectable marker. The gp96-Ig fusion protein coding sequence, for example, can be inserted into a position between the 5′ LTR and 3′ LTR, such that transcription from the 5′ LTR promoter transcribes the cloned DNA. The 5′ LTR contains a promoter (e.g., an LTR promoter), an R region, a U5 region, and a primer binding site, in that order. Nucleotide sequences of these LTR elements are well known in the art. A heterologous promoter as well as multiple drug selection markers also can be included in the expression vector to facilitate selection of infected cells. See, McLauchlin et al., Prog Nucleic Acid Res Mol Biol 1990, 38:91-135; Morgenstern et al., Nucleic Acid Res 1990, 18:3587-3596; Choulika et al., J Virol 1996, 70:1792-1798; Boesen et al., Biotherapy 1994, 6:291-302; Salmons and Gunzberg, Human Gene Ther 1993, 4:129-141; and Grossman and Wilson, Curr Opin Genet Devel 1993, 3:110-114.

Any of the cloning and expression vectors described herein may be synthesized and assembled from known DNA sequences using techniques that are known in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

In some embodiments, the present disclosure utilizes a cell that is transfected with a vector encoding a gp96-Ig fusion protein. Without wishing to be bound by theory, it is believed that administration of gp96-Ig secreting cells triggers robust, antigen-specific CD8 cytotoxic T lymphocyte (CTL) expansion, combined with activation of the innate immune system. Tumor cell-secreted gp96 causes the recruitment of DCs and natural killer (NK) cells to the site of gp96 secretion, and mediates DC activation. Further, the endocytic uptake of gp96 and its chaperoned peptides triggers peptide cross presentation via major MHC class I, as well as strong, cognate CD8 activation independent of CD4 cells.

In some embodiments, expression vectors as described herein can be introduced into host cells for producing secreted vaccine proteins (e.g., gp96-Ig). A variety of techniques are available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003).

Cells may be cultured in vitro or genetically engineered, for example. Host cells can be obtained from normal or affected subjects, including healthy humans, cancer patients, and patients with an infectious disease, private laboratory deposits, public culture collections such as the American Type Culture Collection, or from commercial suppliers.

Exemplary mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the fusion proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, human small cell lung carcinoma cell lines, SCLC #2 and SCLC #7, human lung adenocarcinoma cell line, e.g., AD100, and human prostate cancer cell line, e.g., PC-3.

Cells that can be used for production and secretion of gp96-Ig fusion proteins in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, or granulocytes, various stem or progenitor cells, such as hematopoietic stem or progenitor cells (e.g., as obtained from bone marrow), umbilical cord blood, peripheral blood, fetal liver, etc., and tumor cells (e.g., human tumor cells). The choice of cell type depends on the type of tumor or infectious disease being treated or prevented, and can be determined by one of skill in the art.

Different host cells have characteristic and specific mechanisms for post-translational processing and modification of proteins. A host cell may be chosen which modifies and processes the expressed gene products in a specific fashion similar to the way the recipient processes its heat shock proteins (hsps). For the purpose of producing large amounts of gp96-Ig, it can be preferable that the type of host cell has been used for expression of heterologous genes, and is reasonably well characterized and developed for large-scale production processes. In some embodiments, the host cells are autologous to the patient to whom the present fusion or recombinant cells secreting the present fusion proteins are subsequently administered.

In some embodiments, an expression construct can be introduced into an antigenic cell. As used herein, antigenic cells can include preneoplastic cells that are infected with a cancer-causing infectious agent, such as a virus, but that are not yet neoplastic, or antigenic cells that have been exposed to a mutagen or cancer-causing agent, such as a DNA-damaging agent or radiation, for example. Other cells that can be used are preneoplastic cells that are in transition from a normal to a neoplastic form as characterized by morphology or physiological or biochemical function.

Typically, the cancer cells and preneoplastic cells used in the methods provided herein are of mammalian origin. Mammals contemplated include humans, companion animals (e.g., dogs and cats), livestock animals (e.g., sheep, cattle, goats, pigs and horses), laboratory animals (e.g., mice, rats and rabbits), and captive or free wild animals.

In some embodiments, as mentioned, cancer cells (e.g., human tumor cells) can be used in the methods described herein. In some embodiments, the cell is a human tumor cell. In some embodiments, the cell is an irradiated or live and attenuated human tumor cell. The cancer cells provide antigenic peptides that become associated non-covalently with the expressed gp96-Ig fusion proteins. Cell lines derived from a preneoplastic lesion, cancer tissue, or cancer cells also can be used, provided that the cells of the cell line have at least one or more antigenic determinant in common with the antigens on the target cancer cells. Cancer tissues, cancer cells, cells infected with a cancer-causing agent, other preneoplastic cells, and cell lines of human origin can be used. Cancer cells excised from the patient to whom ultimately the fusion proteins ultimately are to be administered can be particularly useful, although allogeneic cells also can be used. In some embodiments, a cancer cell can be from an established tumor cell line such as, without limitation, an established NSCLC, bladder cancer, melanoma, ovarian cancer, renal cell carcinoma, prostate carcinoma, sarcoma, breast carcinoma, squamous cell carcinoma, head and neck carcinoma, hepatocellular carcinoma, pancreatic carcinoma, or colon carcinoma cell line.

In some embodiments, the fusion protein used allow for the presentation of various tumor cell antigens. For instance, in some embodiments, the vaccine protein fusions (e.g., gp96 fusions) chaperone these various tumor antigens. In some embodiments, the tumor cells secrete a variety of antigens. Illustrative, but non-limiting, antigens that can be secreted and/or presented are: Cancer/testis antigen 1A (CTAG1A) and its immunogenic epitopes CT45A6, CT45A3, CT45A1, CT45A5, sperm autoantigenic protein 17 (SPA17), sperm associated antigen 6 (SPAG6), sperm associated antigen 8 (SPAG8), ankyrin repeat domain 45 (ANKRD45), lysine demethylase 5B (KDM5B), sperm acrosome associated 3 (SPACA3), sperm flagellar 2 (SPEF2), Hemogen (HEMGN), protease, serine 50 (PRSS50), PDZ binding kinase (PBK), Transketolase-like protein 1 (TKTL1), TGFB induced factor homeobox 2 like, X-linked (TGIF2LX), variable charge, X-linked (VCX), chromosome X open reading frame 67 (CXORF67), MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9, GAGE12G, GAGE12F, GAGE12I), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD20, CD22, CD30, CD33, CD37, CD56, CD70, CD74, CD138, AGS16, MUC1, GPNMB, Ep-CAM, PD-L1, PD-L2, and PMSA.

As mentioned above, in some embodiments, the second therapy is chemotherapy. The chemotherapy can be selected from alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above.

In various embodiments in accordance with the present disclosure, the sample from the patient can be selected from a tissue biopsy, tumor biopsy, tumor resection, frozen tumor tissue specimen, lymph node, bone marrow, circulating tumor cells, cultured cells, a formalin-fixed paraffin embedded tumor tissue specimen, and combinations thereof. The biopsy can be selected from a core biopsy, needle biopsy, surgical biopsy, and an excisional biopsy.

Various assays can be used in embodiments in accordance with the present disclosure for analyzing the sample from the patient for a T cell modulatory effect. For example, in some embodiments, the assay is a measurement of cytokine levels, cytokine secretion, surface markers, cytolytic protein secretion, and/or genomic profiles. The assay may employ cytoplasmic dyes, optionally being carboxyfluorescein succinimidyl ester (CFSE). For example, in some embodiments, the assay employs CFSE and measures cell proliferation.

In some embodiments, the assay employs one or more of ELISPOT (enzyme-linked immunospot), intracellular cytokine staining (ICS), fluorescence-activated cell sorting, (FACS), microfluidics, PCR, and nucleic acid sequencing. The assay may also employ analysis of surface markers such as CD45RA/RO isoforms and CCR7 or CD62L expression. In some embodiments, the assay is a measurement of cytokine levels and/or cytokine secretion, and the cytokine is selected from one or more of IFN-γ, TNF, and IL-2.

The described method for treating cancer can be used for treat any of various types of cancer. For example, the cancer can be selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In some embodiments in accordance with the present disclosure, cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micro metastases. Also, included are cells having abnormal proliferation that is not impeded by the immune system (e.g., virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the blood-stream (thereby being a circulating tumor cell) to other sites and tissues in the body.

The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may, thus, be a metastatic breast cancer or a metastatic colon cancer, not liver cancer. The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

Representative cancers and/or tumors of the present invention include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

Pharmaceutical Compositions

In addition, the disclosure also provides pharmaceutical compositions for the present methods of treating cancer, which include an antibody or antigen binding fragment, as described herein, in combination with a pharmaceutically acceptable carrier. A “pharmaceutically acceptable carrier” (also referred to as an “excipient” or a “carrier”) is a pharmaceutically acceptable solvent, suspending agent, stabilizing agent, or any other pharmacologically inert vehicle for delivering one or more therapeutic compounds to a subject (e.g., a mammal, such as a human, non-human primate, dog, cat, sheep, pig, horse, cow, mouse, rat, or rabbit), which is nontoxic to the cell or subject being exposed thereto at the dosages and concentrations employed. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with amino acids include, by way of example and not limitation: water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). Pharmaceutically acceptable carriers also include aqueous pH buffered solutions or liposomes (small vesicles composed of various types of lipids, phospholipids and/or surfactants which are useful for delivery of a drug to a mammal). Further examples of pharmaceutically acceptable carriers include buffers such as phosphate, citrate, and other organic acids, antioxidants such as ascorbic acid, low molecular weight (less than about 10 residues) polypeptides, proteins such as serum albumin, gelatin, or immunoglobulins, hydrophilic polymers such as polyvinylpyrrolidone, amino acids such as glycine, glutamine, asparagine, arginine or lysine, monosaccharides, disaccharides, and other carbohydrates including glucose, mannose or dextrins, chelating agents such as EDTA, sugar alcohols such as mannitol or sorbitol, salt-forming counterions such as sodium, and/or nonionic surfactants such as TWEEN™, polyethylene glycol (PEG), and PLURONICS™.

Pharmaceutical compositions can be formulated by mixing one or more active agents with one or more physiologically acceptable carriers, diluents, and/or adjuvants, and optionally other agents that are usually incorporated into formulations to provide improved transfer, delivery, tolerance, and the like. A pharmaceutical composition can be formulated, e.g., in lyophilized formulations, aqueous solutions, dispersions, or solid preparations, such as tablets, dragées or capsules. A multitude of appropriate formulations can be found in the formulary known to all pharmaceutical chemists: Remington's Pharmaceutical Sciences (18^{th} ed, Mack Publishing Company, Easton, Pa. (1990)), particularly Chapter 87 by Block, Lawrence, therein. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies as described herein, provided that the active agent in the formulation is not inactivated by the formulation and the formulation is physiologically compatible and tolerable with the route of administration. See, also, Baldrick, Regul Toxicol Pharmacol 32:210-218, 2000; Wang, Int J Pharm 203:1-60, 2000; Charman, J Pharm Sci 89:967-978, 2000; and Powell et al. PDA J Pharm Sci Technol 52:238-311, 1998), and the citations therein for additional information related to formulations, excipients and carriers well known to pharmaceutical chemists.

Pharmaceutical compositions include, without limitation, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Compositions and formulations can contain sterile aqueous solutions, which also can contain buffers, diluents and other suitable additives (e.g., penetration enhancers, carrier compounds and other pharmaceutically acceptable carriers). Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the polypeptide components within the compositions provided herein. The formulations can be sterilized if desired.

In some embodiments, a composition containing an antibody or antigen binding fragment as used herein can be in the form of a solution or powder with or without a diluent to make an injectable suspension. The composition may contain additional ingredients including, without limitation, pharmaceutically acceptable vehicles, such as saline, water, lactic acid, mannitol, or combinations thereof, for example.

In one aspect, the disclosure provides a method of making an anti-TNFRSF25 antibody or antigen binding fragment thereof, comprising: a) providing the host cell as described herein; b) culturing said host cell under conditions wherein said antibody is expressed; and c) recovering said antibody from the host cell.

Any appropriate method can be used to administer an antibody or antigen binding fragment as described herein to a mammal. Administration can be, for example, parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). In some embodiments, administration can be topical (e.g., transdermal, sublingual, ophthalmic, or intranasal), pulmonary (e.g., by inhalation or insufflation of powders or aerosols), or oral. In addition, a composition containing an antibody or antigen binding fragment as described herein can be administered prior to, after, or in lieu of surgical resection of a tumor.

A composition containing an anti-TNFRSF25 antibody or antigen binding fragment thereof can be administered to a mammal in any appropriate amount, at any appropriate frequency, and for any appropriate duration effective to achieve a desired outcome. For example, an anti-TNFRSF25 antibody or antigen binding fragment thereof can be administered to a subject in an amount effective to stimulate proliferation of T cells in vitro or in vivo (e.g., human, murine, hamster, or macaque T cells, including CD8⁺ T cells and/or CD4⁺FoxP3⁺ regulatory T cells), to stimulate apoptosis of tumor cells that express TNFRSF25, to reduce tumor size, or to increase progression-free survival of a cancer patient. In some embodiments, an anti-TNFRSF25 antibody or antigen binding fragment thereof can be administered at a dosage of about 0.1 mg/kg to about 10 mg/kg (e.g., about 0.1 mg/kg to about 1 mg/kg, about 1 mg/kg to about 5 mg/kg, or about 5 mg/kg to about 10 mg/kg), and can be administered once every one to three weeks (e.g., every week, every 10 days, every two weeks, or every three weeks).

In some cases, a composition containing an anti-TNFRSF25 antibody or antigen binding fragment thereof as described herein can be administered to a subject in an amount effective to increase proliferation of T cells (e.g., by at least about 10 percent, about 20 percent, about 25 percent, about 50 percent, about 60 percent, about 70 percent, about 75 percent, about 80 percent, about 90 percent, about 100 percent, or more than 100 percent), as compared to the “baseline” level of T cell proliferation in the subject prior to administration of the composition, or as compared to the level of T cell proliferation in a control subject or population of subjects to whom the composition was not administered. The T cells can be, for example, CD8⁺ T cells, or CD4⁺FoxP3⁺ regulatory T cells. Any suitable method can be used to determine whether or not the level of T cell proliferation is increased in the subject. Such methods can include, without limitation, flow cytometry analysis of antigen specific T cells (e.g., flow cytometry analysis of the proportion of antigen specific CD8⁺ T cells as a fraction of the total CD8⁺ T cell pool), analysis of cell proliferation markers (e.g., expression of Ki67) in CD8⁺ T cells, increased counts of CD8⁺ T cells, or increased proportions of individual TCR sequences of a particular clone of CD8⁺ T cells.

The present disclosure also provides methods for treating cancer by promoting apoptosis of TNFRSF25-expressing tumor cells in a subject, by treating the subject with an antibody, antigen-binding fragment, or composition as described herein. In some cases, a composition containing an antibody or antigen binding fragment as provided herein can be administered to a subject (e.g., a cancer patient) in an amount effective to increase apoptosis of TNFRSF25 expressing tumor cells (e.g., by at least about 10 percent, about 20 percent, about 25 percent, about 50 percent, about 60 percent, about 70 percent, about 75 percent, about 80 percent, about 90 percent, about 100 percent, or more than 100 percent), as compared to the “baseline” level of tumor cell apoptosis in the subject prior to administration of the composition, or as compared to the level of tumor cell apoptosis in a control subject or population of subjects to whom the composition was not administered. Any suitable method can be used to determine whether or not the level of tumor cell apoptosis is increased in the subject. This can include, for example, radiologic techniques such as CT or MRI, with or without contrast that indicates the presence of a necrotic or apoptotic tumor, biopsy of a tumor sample indicating increased tumor cell death, caspase induction within tumor cells, elimination of detectable tumor lesions by radiologic, or surgical or physical examination.

Methods for treating a subject (e.g., a human patient) with cancer, including solid tumors and leukemias/lymphomas) also are provided herein. In some cases, a composition containing an antibody or antigen binding fragment as described herein can be administered to a subject having cancer in an amount effective to reduce the progression rate of the cancer (e.g., by at least about 10 percent, about 20 percent, about 25 percent, about 50 percent, about 60 percent, about 70 percent, about 75 percent, about 80 percent, about 90 percent, or more than 90 percent), as compared to the rate of cancer progression in the subject prior to administration of the composition, or as compared to the rate of cancer progression in a control subject or population of subjects to whom the composition was not administered. In some embodiments, the progression rate can be reduced such that no additional cancer progression is detected. Any appropriate method can be used to determine whether or not the progression rate of cancer is reduced. For skin cancer (e.g., melanoma), for example, the progression rate can be assessed by imaging tissue at different time points and determining the amount of cancer cells present. The amounts of cancer cells determined within tissue at different times can be compared to determine the progression rate. After treatment as described herein, the progression rate can be determined again over another time interval. In some cases, the stage of cancer after treatment can be determined and compared to the stage before treatment to determine whether or not the progression rate has been reduced.

A composition containing an antibody or antigen binding fragment as described herein also can be administered to a subject having cancer under conditions where progression-free survival is increased (e.g., by at least about 10 percent, about 20 percent, about 25 percent, about 50 percent, about 60 percent, about 70 percent, about 75 percent, about 80 percent, about 90 percent, about 100 percent, or more than 100 percent), as compared to the median progression-free survival of corresponding subjects having untreated cancer or the median progression-free survival of corresponding subjects having cancer and treated with other therapies (e.g., chemotherapeutic agents alone). Progression-free survival can be measured over any length of time (e.g., one month, two months, three months, four months, five months, six months, or longer).

An effective amount of a composition containing a molecule as used herein can be any amount that has a desired defect (e.g., stimulates proliferation of CD8⁺ T cells, stimulates apoptosis of TNFRSF25-expressing tumor cells, stimulates or elicits an immune response in a subject, reduces tumor size, reduces the progression rate of cancer, increases progression-free survival of a cancer patient, or increases the median time to progression without producing significant toxicity). Dosages can vary depending on the relative potency of individual polypeptides (e.g., antibodies and antigen binding fragments), and can generally be estimated based on EC50 found to be effective in in vitro and in vivo animal models. Typically, dosage is from 0.01 μg to 100 g per kg of body weight. For example, an effective amount of an antibody or antigen binding fragment thereof can be from about 0.1 mg/kg to about 50 mg/kg (e.g., about 0.4 mg/kg, about 2 mg/kg, about 5 mg/kg, about 10 mg/kg, about 20 mg/kg, about 30 mg/kg, about 40 mg/kg, or about 50 mg/kg), or any range there between, such as about 0.1 mg/kg to about 10 mg/kg, about 0.4 mg/kg to about 20 mg/kg, about 2 mg/kg to about 30 mg/kg, or about 5 mg/kg to about 40 mg/kg. If a particular subject fails to respond to a particular amount, then the amount of the antibody or antigen binding fragment thereof can be increased by, for example, two fold. After receiving this higher concentration, the subject can be monitored for both responsiveness to the treatment and toxicity symptoms, and adjustments made accordingly. The effective amount can remain constant or can be adjusted as a sliding scale or variable dose depending on the subject's response to treatment. Various factors can influence the actual effective amount used for a particular application. For example, the frequency of administration, duration of treatment, use of multiple treatment agents, route of administration, and severity of the cancer may require an increase or decrease in the actual effective amount administered.

The frequency of administration can be any frequency that, for example, stimulates proliferation of CD8⁺ T cells, stimulates apoptosis of TNFRSF25 expressing tumor cells, reduces tumor size, reduces the progression rate of cancer, increases progression-free survival of a cancer patient, or increases the median time to progression without producing significant toxicity. For example, the frequency of administration can be once or more daily, biweekly, weekly, monthly, or even less. The frequency of administration can remain constant or can be variable during the duration of treatment. A course of treatment can include rest periods. For example, a composition containing an antibody or antigen binding fragment as used herein can be administered over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the cancer may require an increase or decrease in administration frequency.

An effective duration for administering a composition used herein can be any duration that stimulates proliferation of CD8⁺ T cells, stimulates apoptosis of TNFRSF25 expressing tumor cells, reduces tumor size, reduces the progression rate of cancer, increases progression-free survival of a cancer patient, or increases the median time to progression without producing significant toxicity. Thus, an effective duration can vary from several days to several weeks, months, or years. In general, the effective duration for the treatment of cancer can range in duration from several weeks to several months. In some cases, an effective duration can be for as long as an individual subject is alive. Multiple factors can influence the actual effective duration used for a particular treatment. For example, an effective duration can vary with the frequency of administration, effective amount, use of multiple treatment agents, route of administration, and severity of the cancer.

After administering an TNFRSF25 agonistic antibody or antigen binding fragment thereof and a second therapy as provided herein to a cancer patient, the patient can be monitored to determine whether or not the cancer was treated. For example, a subject can be assessed after treatment to determine whether or not the progression rate of the cancer has been reduced (e.g., stopped). Any method, including those that are standard in the art, can be used to assess progression and survival rates.

A method for using an TNFRSF25 agonistic antibody or antigen binding fragment thereof and a second therapy can be combined with known methods of treatment for cancer, for example, either as combined or additional treatment steps, or as additional components of a therapeutic formulation. For example, enhancing a host's immune function can be useful to combat tumors. Methods can include, without limitation, APC enhancement, such as by injection into a tumor of DNA encoding foreign MHC antigens (including tumor antigens, mutation derived antigens, or other antigens), or transfecting biopsied tumor cells with genes that increase the probability of immune antigen recognition (e.g., immune stimulatory cytokines, GM-CSF, or co-stimulatory molecules B7.1, B7.2) of the tumor. Other methods can include, for example, solubilization of specific tumor antigens into depot or sustained release preparations, transfection of allogeneic tumor cells with adjuvant proteins or antigen carrier proteins, transfection of allogeneic tumor cells with immune stimulatory proteins such as alpha galactosylceramide, incorporation of specific tumor antigens into virus-derived vaccine regimens, incorporation of specific tumor antigens into Listeria derived vaccine regimens, adoptive cellular immunotherapy (including chimeric antigen receptor transfected T cells), or treatment with activated tumor-specific T cells (including ex vivo expanded tumor infiltrating lymphocytes). Adoptive cellular immunotherapy can include isolating tumor-infiltrating host T-lymphocytes and expanding the population in vitro (e.g., by stimulation with IL-2). The T cells then can be re-administered to the host. Other treatments that can be used in combination with an antibody or antigen-binding fragment as provided herein include, for example, radiation therapy, chemotherapy, hormonal therapy, and the use of angiogenesis inhibitors. Further combination partners that may be useful include checkpoint inhibitors (e.g., anti-PD1/L1, anti-CTLA-4, anti-LAG3, anti-B7-H3, anti-B7-H4, anti-TIM3, anti-TIGIT, anti-CD47, anti-TMIGD2, anti-BTLA, anti-CEACAM, or anti-GARP), other costimulatory antibodies (e.g., anti-OX40, anti-ICOS, anti-CD137, anti-GITR, or anti-CD40), cancer vaccines (e.g., virus based vaccines, peptide vaccines, whole-cell vaccines, or RNA based vaccines), and targeted agents [e.g., HERCEPTIN® (trastuzumab), TARCEVA® (erlotinib), AVASTIN® (bevacizumab), or IMBRUVICA® (ibrutinib)].

Thus, in some embodiments, an anti-TNFRSF25 antibody or antigen binding fragment thereof can be used in combination with one or more additional monoclonal antibodies that inhibit binding of PD-L1 to PD-1, inhibit binding of CTLA-4 to CD80 or CD86, or activate signaling via the TNFRSF4, TNFRSF9, or TNFRSF18 pathways, for example. In some embodiments, the antibody against PD-1 is selected from nivolumab (OPDIVO), pembrolizumab (KEYTRUDA), pidilizumab, cemiplimab, AGEN2034, AMP-224, AMP-514, PDR001. In some embodiments, the antibody against PD-L1 is selected from Atezolizumab (TECENTRIQ), avelumab (BAVENCIO), durvalumab (IMFINZI), BMS-936559, and CK-301. In some embodiments, the antibody against CTLA-4 is selected from ipilimumab (YERVOY), tremelimumab, AGEN1884, and RG2077.

This also can include administration with another antibody, fusion protein, or small molecule that binds a specific target on a tumor cell (e.g., combinations with monoclonal antibodies that bind targets such as CD20, Her2, EGFRvIII, DR4, DR5, VEGF, CD39, and CD73). An anti-TNFRSF25 antibody or antigen binding fragment also can be used in combination with a cancer vaccine approach to enhance the activation of tumor antigen specific T cells in a cancer patient. In addition, an anti-TNFRSF25 antibody or antigen binding fragment thereof can be used after administration of autologous or allogeneic T or NK cells engineered to express a chimeric T cell receptor that recognizes a specific tumor antigen. Further, an anti-TNFRSF25 antibody or antigen binding fragment thereof can be used in combination with specific chemotherapy or radiation therapy strategies as a method to expand tumor specific T cells and enhance the activity of either approach as a monotherapy in a cancer patient.

When one or more conventional therapies are combined with treatment using an anti-TNFRSF25 antibody or antigen binding fragment as used herein for treating cancer, for example, the conventional therapy(ies) can be administered prior to, subsequent to, or simultaneously with administration of the anti-TNFRSF25 antibody or antigen binding fragment thereof. For example, a PD-1 blocking antibody can be administered to a patient prior to administration of a TNFRSF25 agonist antibody. Such a regimen can be cycled over a period of weeks, months, or years, for example. Alternatively, a PD-1 blocking antibody can be administered at the same time or after administration of a TNFRSF25 agonist antibody. Such a regimen also can be cycled over a period of weeks, months, or years. In some embodiments, combination therapies that are repeatedly administered over a period of time can include two or more of the above administration strategies.

In some embodiments, an anti-TNFRSF25 antibody or antigen binding fragment as used herein can be used during an in vitro assay or manufacturing process as a method for stimulating proliferation of tumor infiltrating lymphocytes isolated from a cancer patient, or to stimulate proliferation of chimeric antigen receptor expressing T cells being expanded in vitro and intended for subsequent infusion for the treatment of a cancer patient.

Also provided herein are articles of manufacture containing an antibody or antigen binding fragment as described herein, or a pharmaceutical composition containing the antibody or antigen binding fragment thereof. The antibody or pharmaceutical composition can be within a container (e.g., a bottle, vial, or syringe). The article of manufacture also can include a label with directions for reconstituting and/or using the antibody, antigen binding fragment, or composition. In some embodiments, an article of manufacture can include one or more additional items (e.g., one or more buffers, diluents, filters, needles, syringes, and/or package inserts with further instructions for use). An article of manufacture also can include at least one additional agent for treating cancer. For example, an article of manufacture as provided herein can contain an agent that targets CTLA-4, PD-1, PD-L1, LAG-3, Tim-3, TNFRSF4, TNFRSF9, TNFRSF18, CD27, CD39, CD47, CD73, or CD278. In some embodiments, an article of manufacture can contain an A2A receptor antagonist or a TGF-beta antagonist. In some embodiments, an article of manufacture can include a B7 family costimulatory molecule (e.g., CD28 or CD278) or a TNF receptor superfamily costimulatory molecule (e.g., TNFRSF4, TNFRSF9, or TNFRSF18), a chemotherapeutic agent, or an anti-tumor vaccine composition.

In order that the invention disclosed herein may be more efficiently understood, examples are provided below. It should be understood that these examples are for illustrative purposes only and are not to be construed as limiting the invention in any manner.

### EXAMPLES

### Example 1: Human PTX-35 Targets Engagement and T Cell Expansion in Non-Human Primates

A two-week toxicity study was conducted in cynomolgus monkeys, where human PTX-35 was provided once every 2-weeks (i.e., day 1 and day 15) for a total of 2 doses, intravenously, via bolus injection. The study was followed by a 3 or 4-week recovery period. The objectives were, inter alia, to determine the potential toxicity of PTX-35 and to evaluate the potential reversibility of any findings following a 3 or 4 week dose-free period.

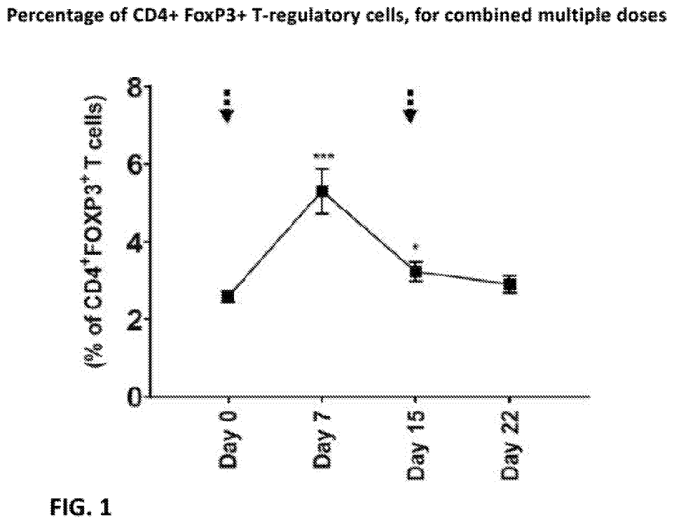

In the present study all doses were grouped together, for analysis purposes, to measure the pharmacodynamic (PD) response in blood leukocytes. Treatment of monkeys with PTX-35, expanded CD4⁺FOXP3⁺ regulatory T cells, seven-days post injection, as shown in FIG. 1 (p<0.001), where arrows indicate days of PTX-35 infusions (dose).

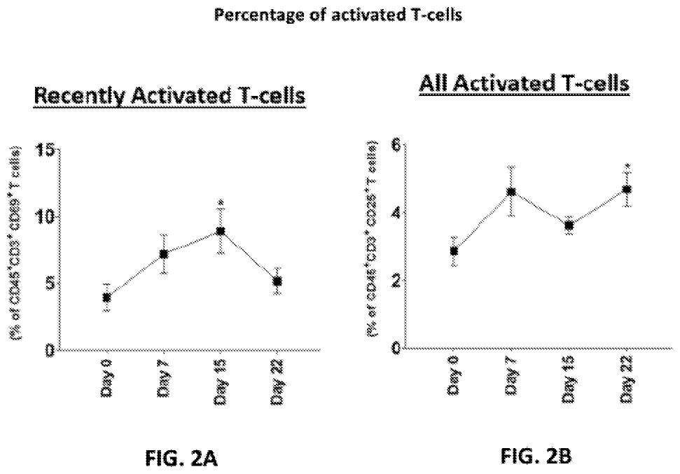

FIG. 2A illustrates the increase in the percent of recently activated T cells (% of CD45⁺CD3⁺CD69⁺ T cells) that peaks on day 15, and FIG. 2B illustrates the increase in the percent of all activated T cells (% of CD45⁺CD3⁺CD25⁺ T cells), which peaks on day 7 and then on day 22.

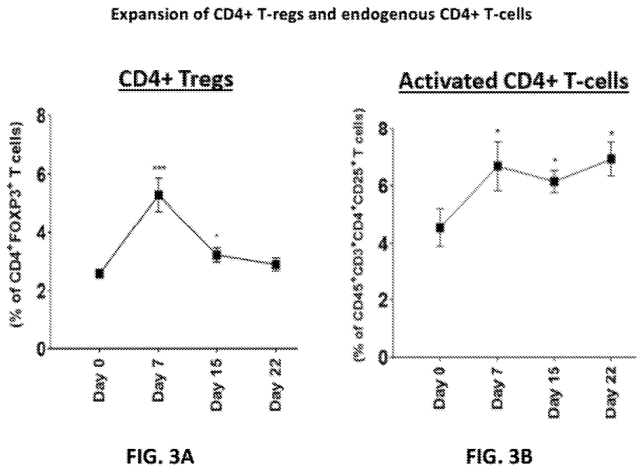

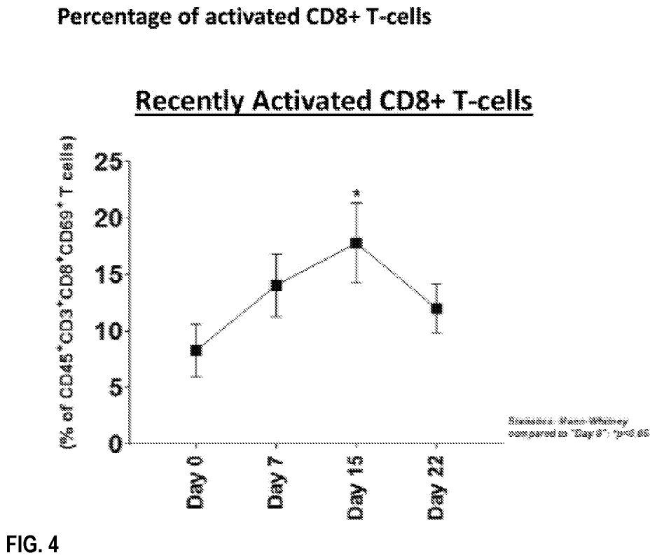

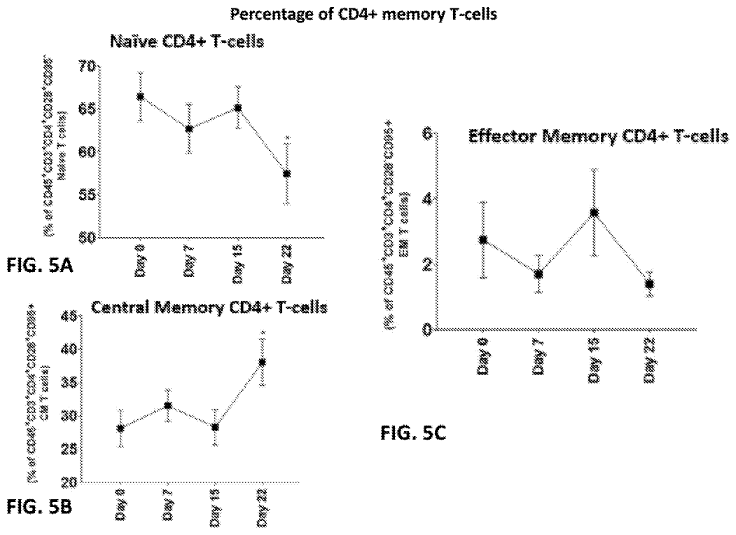

FIGS. 3A and 3B illustrate expansion of T-regs and endogenous CD4⁺ T cells, respectively. FIG. 3A illustrates the increase in the percent of CD4⁺ T-regs (% of CD4⁺FOXP3⁺ T cells) in the peripheral blood, which peaks on day 7. FIG. 3B illustrates the increase in the percent of activated CD4⁺ T cells (% of CD45⁺CD3⁺CD4⁺CD25⁺ T cells), which peaks on day 7 and then on day 22. FIG. 4 illustrates the increase in the percent of recently activated CD8⁺ T cells (% of CD45⁺CD3⁺CD8⁺CD69⁺ T cells) that peaks on day 15. The CD8⁺ T cells expand to the extent similar to the expansion of CD4⁺ T cells. FIG. 4 demonstrates that PTX-35 modulates activation state of T cells in non-human primates.

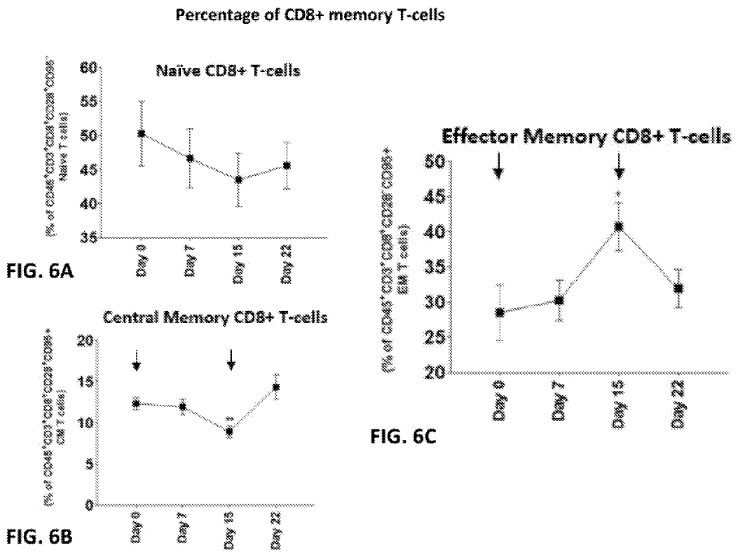

In the present study, a differential expansion of memory T cell subsets, effector memory and central memory T cells, is observed, as shown in FIGS. 5A-5C and 6A-6C. FIG. 5A shows the percentage of naïve CD4⁺ T cells (% of CD45⁺CD3⁺CD4⁺CD28⁺CD95⁺ naïve T cells), FIG. 5B shows the percentage of central memory CD4⁺ T cells (% of CD45⁺CD3⁺CD4⁺CD28⁺CD95⁺ CM (central memory) T cells), and FIG. 5C shows the percentage of effector memory CD4⁺ T cells (% of CD45⁺CD3⁺CD4⁺CD28⁺CD95⁺ EM (effector memory) T cells). FIGS. 5A-5C illustrate that central memory CD4⁺ T cell subsets show significant expansion after the second dose of PTX-35, at the expense of effector memory CD4⁺ T cells. Thus, for CD4⁺ T cells, the differential expansion of effector memory and central memory T cells provides an increase in a ratio of CD4⁺ central memory T cells to CD4⁺ effector memory T cells.

FIG. 6A shows the percentage of naïve CD8⁺ T cells (% of CD45⁺CD3⁺CD8⁺CD28⁺CD95⁺ naïve T cells), FIG. 6B shows the percentage of central memory CD8⁺ T cells (% of CD45⁺CD3⁺CD8⁺CD28⁺CD95⁺ CM (central memory) T cells), and FIG. 6C shows the percentage of effector memory CD8⁺ T cells (% of CD45⁺CD3⁺CD8⁺CD28⁺CD95⁺ EM (effector memory) T cells). FIGS. 6A-6C illustrate that central memory CD8⁺ T cell populations decrease upon stimulation with PTX-35, as effector memory CD8⁺ T cells increase. Thus, CD8⁺ T cells, the differential expansion of effector memory and central memory T cells provides an increase in a ratio of CD8⁺ effector memory to CD8⁺ central memory T cells. This expansion of effector memory CD8⁺ T cell subsets suggests combination advantages of PTX-35 with cancer vaccines or antigen-driven immunotherapy.

FIGS. 5A-5C and 6A-6C demonstrate that PTX-35 modulates memory state of T cells in non-human primates.

### Example 2: In Vitro Analysis of Human PTX-35 Regarding Costimulation to Provide Antigen Experienced T Cells in Human PBMC Cultures

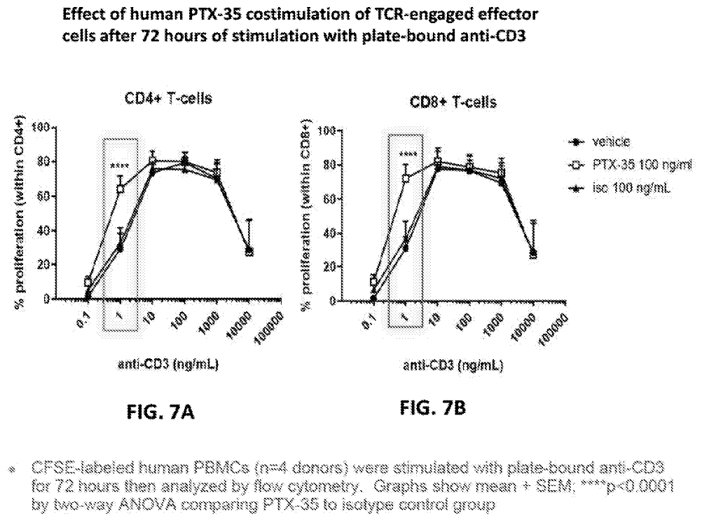

Carboxyfluorescein succinimidyl ester (CFSE)-labeled human peripheral blood mononuclear cells (PBMCs) from four different donors (n=4 biological replicates) were stimulated for 72 hours with serial dilutions of plate-bound anti-CD3 and 100 ng/mL of human PTX-35 or isotype control (hIgG2). Cells were then stained for viability, and CFSE dilution was assessed by flow cytometry with gating on CD4 versus CD8 within the viable cell population. FIG. 7A illustrates the proportion of CD4⁺ T cells that divided, and FIG. 7B illustrates the proportion of CD8⁺ T cells that divided. The graphs in FIGS. 7A and 7B show mean+SEM, and “****” denotes p<0.0001 by two-way ANOVA comparing PTX-35 to isotype control group. FIGS. 7A and 7B illustrate that human PTX-35 costimulation of TCR-engaged effector cells after stimulation with plate-bound anti-CD3 drives proliferation in human PBMCs. The results demonstrate that PTX-35 is a co-stimulatory molecule (anti-CD3 provides an artificial Ag signal), and that TNFRSF25 engagement in combination with antigen stimulation can enhance CD4⁺ and CD8⁺ T cell responses.

### Example 3: Successful Generation of a Mouse-Human Surrogate Antibody for PTX-35

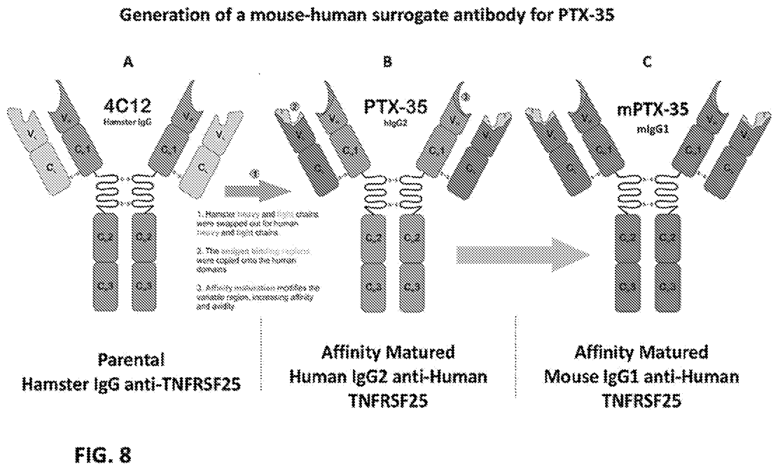

PTX-35 is a humanized, affinity matured, IgG2 monoclonal antibody derived from the hamster antibody 4C12 (see FIG. 8), a product of immunization and immortalization as a hybridoma cell line. PTX-35 is a functional agonist of human TNFRSF25. FIG. 8 illustrates schematically that a “parental” hamster IgG anti-TNFRSF25 antibody (4C12) was modified to generate affinity matured human IgG2 anti-human TNFRSF25 (PTX-35), as shown in panels A and B. In particular, hamster heavy and lights chains were swapped out for human heavy and light chains, the antigen binding regions were copied from 4C12 onto human domains, and affinity maturation was used to modify the variable regions of the heavy and the light chains, increasing affinity and avidity, as shown in FIG. 8.

Human PTX-35 is an IgG2, which shows poor binding affinity (and agonist activity) against mouse Fc-gamma receptors. See Dekkers et al. Affinity of human IgG subclasses to mouse Fc gamma receptors. MABS, 2017, Volume 9 (5): 767-773. Accumulated field knowledge in designing monoclonal antibodies targeting the TNF-family of cell surface receptors, such as 4-1BB, OX40, TNFR1 and CD40, has focused on the importance of target trimerization to allow for proper signaling. See Mayes et al. The promise and challenges of immune agonist antibody development in cancer. Nature Reviews Drug Discovery (2018), 17:509-527. A series of small in vitro investigations using 4C12 and PTX-35 demonstrated that (1) the affinity-matured variable domain of PTX-35 is different than 4C12, and results in a greater affinity for TNFRSF25, and (2) cross-linking plays an essential role in ability of a mAb to trimerize bound target. Thus, deficiencies in the Fc-affinity for Fc-receptors in vivo will compromise mechanism and thus pharmacodynamic effect.

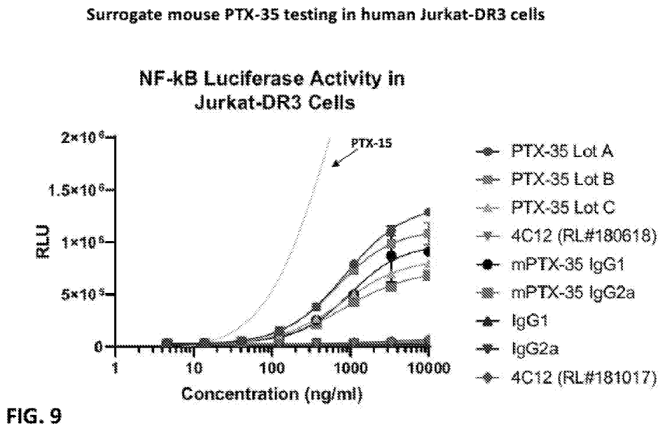

To overcome the above challenge, the inventors generated two mouse-human surrogate antibodies for human PTX-35 to produce a “tool reagent” that could be used for experimental purposes. Specifically, human PTX-35 variable domains were cloned onto a mouse backbone, IgG1 (similar to human IgG4, limited effector Fc function), shown in FIG. 8 (panel C), and mouse IgG2a (similar to human IgG1, greatest effector Fc function, CDC/ADCC). The CDRs of the human PTX-35 are preserved on mPTX-35. When the IgG1 and IgG2a PTX-35 mouse surrogates were tested in the Jurkat-DR3 assay, both induced DR3 signaling similar to that seen for human PTX-35, as shown in FIG. 9, discussed in more detail below. The surrogate mouse-human antibodies are generated to include the anti-human variable domain but on a mouse Ig heavy chain, to allow activity in mice. When FIR (Foxp3-RFP) mice were provided these antibodies at single 10 mg/kg doses, the mouse IgG1 version (IgG1-PTX-35 or mPTX-35) provided the most prominent in vivo expansion of Tregs in the FIR mice (not shown). This data illustrates that the generated mPTX-35 is a working surrogate antibody that may be combined with mouse HS-110 vaccines to develop a clinical path for cancer vaccine combinations.

FIG. 9 shows testing of surrogate mouse PTX-35 (mPTX-35) in human Jurkat cells expressing human DR3, also referred to as Jurkat-DR3 cells. The Jurkat-DR3 cells are a human T cell line that express the luciferase gene under the control of the NF-κB promoter that is activated upon stimulation of the death receptor 3 (DR3) signaling pathway. The purpose of this experiment was to test the effect of PTX-35 lots on the DR3 pathway. FIG. 9 shows NF-κB luciferase activity, in relative luminometer units (RLUs), depending on a concentration of the tested antibodies.

Jurkat-DR3 cells were treated with three different lots of human PTX-35 (clinical lot A (blue line), clinical lot B (red line), and clinical lot C (green line)), two lots of 4C12 (RL #180618 (magenta line) and RL #181017 (maroon line)), mPTX-35 with IgG1 isotype (black line), and mPTX-35 with IgG2a isotype (brown line). IgG1 and IgG2a (shown in dark blue and purple lines, respectively) were used as negative controls, and PTX-15 (human TL1A-Ig), shown with the thin blue line, was used as a positive control. Each antibody was diluted to a concentration of 30 ug/ml, which is three times the amount of the desired highest final concentration in the assay, and 100 ul (microliter) of each was transferred in triplicate to a 96-well plate. Three-fold serial dilutions were then performed for each antibody down to the lowest concentration of 4.57 ng/ml. Twenty-five microliters of each dilution was then transferred to a new 96-well assay plate. The Jurkat-DR3 cells were counted and resuspended to a concentration of 2×10⁶ cells/ml and 50 ul of cells (1×10⁵ cells) were dispensed into each assay well containing the antibody dilutions. This brought the final concentration of the antibodies down to a range of 10 μg/ml to 1.52 ng/ml. The treated cells were incubated for 6 hours at 37° C. with 5% CO₂. At the end of the 6-hour incubation, the assay plate was removed from the incubator and allowed to equilibrate to room temperature for 10 minutes. After equilibration, 75 ul of Promega Bio-Glo reagent was added to each well and incubated for 5 minutes. Luciferase expression was then quantified on a Promega GloMax plate reader. In FIG. 9, the graphs show mean±SD.

FIG. 9 illustrates that human Jurkat-DR3 show similar activity between human PTX-35 (clinical lots A, B, C) and mouse PTX-35 (mPTX-35) with IgG1 or IgG2a isotypes. Also, as mentioned above, the IgG1 and IgG2a PTX-35 mouse surrogates both induced DR3 signaling similar to human PTX-35.

### Example 4: Use of Radiation Model to Evaluate the Ability of TNFRSF25 and TNFRSF25+CTLA-4 Inhibitor to Decrease Tumor Growth

The purpose of this experiment was to use TNFRSF25 agonism using the mouse surrogate mAb, 4C12, in combination with a standard of care therapy, such as radiation, in mediating tumor growth. TNFRSF25 agonism was also evaluated in combination with CTLA-4 inhibitor.

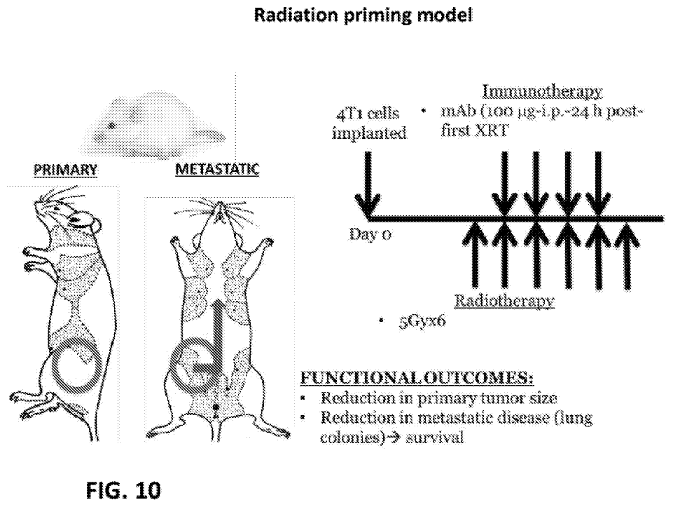

FIG. 10 shows a setup of the in vivo experiments using a radiation priming model. The experiments were conducted as follows:

Day 0: 4T1 tumor cells were injected in the fat pad of Balb/c mice.

Day 7-13: Mice were radiated with 5 Gy radiation for 6 days (5×6) amounting to a cumulative radiation dosage of 30 Gy.

Day 8: The following three series of treatment regimens represent independent experiments conducted serially (all antibodies were administered intraperitoneally at a dose of 100 μg/mouse):

a) Series 1
a. 5×6 radiation alone
b. 5×6 radiation+ISOtype control
c. 5×6 radiation+one-time dose of TNFRSF25 agonist (4C12);

b) Series 2
a. 5×6 radiation alone
b. 5×6 radiation+ISOtype control
c. 5×6 radiation+one-time dose of TNFRSF25 agonist (4C12)
d. 5×6 radiation+one-time dose of TNFRSF25 agonist+CTLA4 inhibitor
e. 5×6 radiation+CTLA4 inhibitor; and

c) Series 3
a. 5×6 radiation alone
b. 5×6 radiation+ISOtype control
c. 5×6 radiation+one-time dose of TNFRSF25 agonist (4C12)
d. 5×6 radiation+one-time dose of TNFRSF25 agonist+anti-PD-1
e. 5×6 radiation+anti-PD-1.

Tumor growth curves were measured at regular intervals. The functional outcome of this experiment was to evaluate the efficacy of the combination therapy in controlling tumor growth.

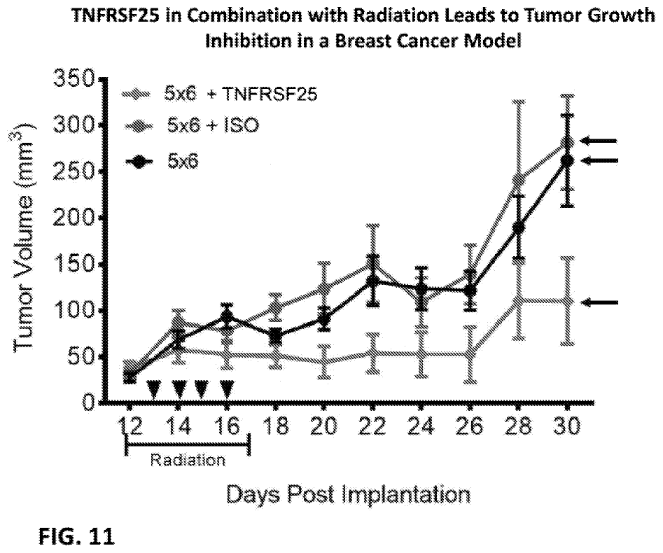

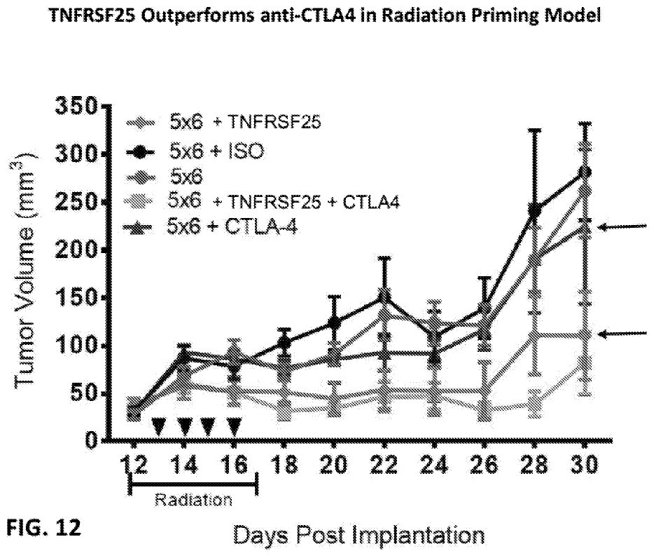

FIG. 11 shows results of the murine breast cancer model study (Series 1), where cumulative tumor growth curves were followed over the course of 30 days between the following treatment groups: Radiation alone, Radiation+Isotype, Radiation+TNFRSF25. Arrows indicate the time treatment was administered (radiation±agonist). FIG. 12 shows results of the mouse breast cancer model study (Series 2), where cumulative tumor growth curves are followed over the course of 30 days between the following groups: Radiation alone (5×6), Radiation+Isotype (5×6+ISO), Radiation+TNFRSF25 (5×6+TNFRSF25), Radiation+CTLA+TNFRSF25 (5×6+TNFRSF25+CTLA), and Radiation+CTLA-4 (5×6+CTLA) treatment groups. Arrows indicate the time treatment was administered (radiation±agonists). In FIGS. 11 and 12, the graphs show the tumor volume (in mm³) versus days post-implantation, in an in vivo study that uses a breast cancer model.

As shown in FIG. 11, while no treatment (“5×6”) and radiation+isotype (“5×6+ISO”) control mice showed increased tumor progression over the course of 30 days, it was observed that combination of radiation together with TNFRSF25 agonism (“5×6+TNFRSF25”) lead to a drastic decrease in tumor size with the average tumor size of 100 mm² on day 30, compared to its control counterparts that exhibited tumor sizes that were 2.5 fold higher at 250 mm² on day 30. Thus, the present study demonstrates that TNFRSF25 in combination with radiation leads to tumor growth inhibition.

FIG. 12 illustrates that, upon evaluating the treatment study involving radiation+CTLA-4 inhibitor (“5×6+CTLA-4”) vs radiation+TNFRSF25 agonist, it was observed that the addition of TNFRSF25 agonist to radiotherapy (either alone (“5×6+TNFRSF25”) or in combination with CTLA-4 inhibitor (“5×6+TNFRSF25+CTLA-4”)) showed a striking decrease in tumor growth curves, with an average tumor growth curve of 50 mm² at day 30 compared to a 2-fold increase in tumor growth with CTLA-4 inhibitor (200 mm² on day 30). FIG. 12 illustrates that TNFRSF25 outperforms anti-CTLA4 in the radiation priming model.

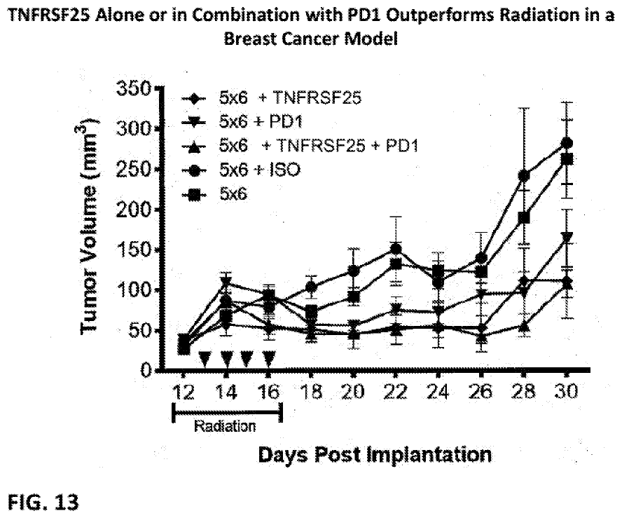

FIG. 13 illustrates that, upon evaluating the treatment study involving radiation+PD1 inhibitor (“5×6+PD1”) vs radiation+TNFRSF25 agonist (“5×6+TNFRSF25”) vs radiation+TNFRSF25 agonist+PD1 inhibitor (“5×6+TNFRSF25+PD1”), it was observed that the addition of TNFRSF25 agonist to radiotherapy, either alone or in combination with PD1 inhibitor, yielded a reduction in tumor volume versus radiation alone (100-150 mm² vs. 270 mm², respectively).

### Example 5: Study of T Cell Expansion Using a Combination of mHS-110 and mPTX

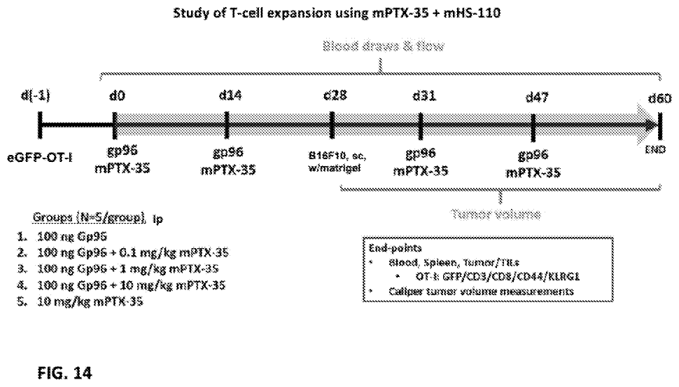

The objective of this study, illustrated in FIGS. 14-20, was to analyze whether TNFRSF25 agonist antibodies, such as mPTX-35 (mouse-IgG1-PTX-35, see FIGS. 8 and 9) in combination with cancer vaccines can be used for synergistic antigen-specific T cell expansion and tumor cell killing. HS-110 (Viagenpumatucel-L) is an allogeneic tumor cell vaccine expressing a recombinant secretory form of the heat shock protein gp96 fusion (gp96-Ig). More specifically, the purpose of this study was to test whether the combination of mPTX-35 with a gp96-Ig expressing allogenic mouse cell line, mHS-110, can enhance the expansion of antigen specific CD8⁺ T cells and delay tumor growth, using a prophylactic challenge model. Also, the goal was to determine whether DR3-agonism, in the context of antigen challenge, enhances T-cell expansion, using mPTX-35 rather than 4C12. In other words, the inventors assessed whether mouse PTX-35 (mPTX-35) (generated as shown, e.g., in FIG. 8) can be used, as a surrogate for human PTX-35, for T cell expansion and delay or reduction in tumor growth, in the same manner as hamster 4C12 antibody.

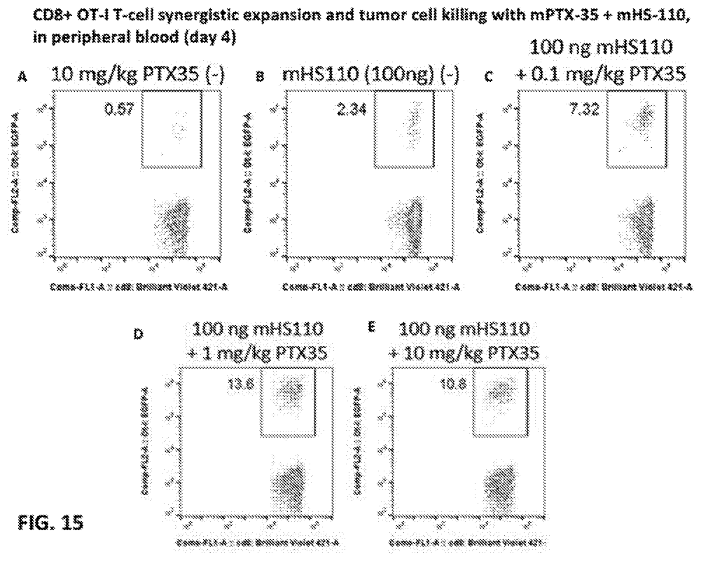

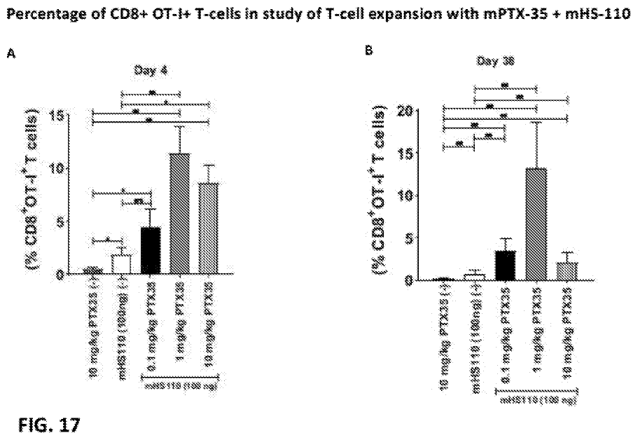

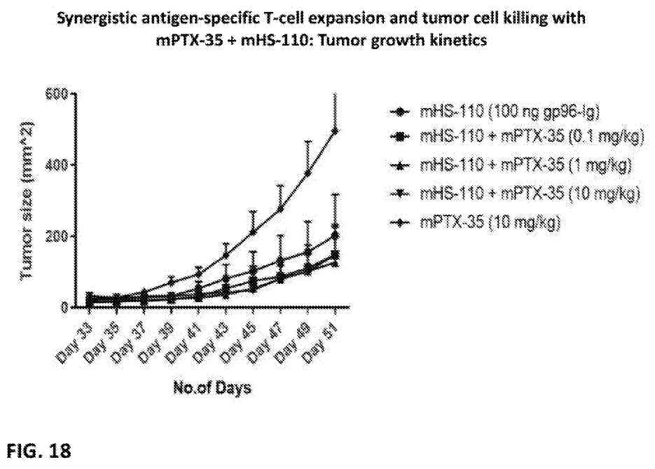

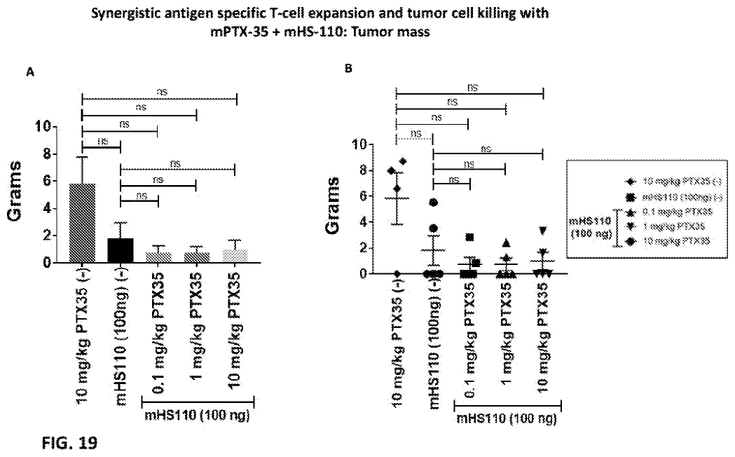

In the present study, a dose of mouse HS-110 (mHS-110, i.e. gp96-Ig) was 100 ng released in a 24-hour period per million cells in 1 mL of medium (which was defined as sub-optimal), and this dose was used in combination with mPTX-35 at the 0.1 mg/kg, 1 mg/kg and 10 mg/kg doses. FIG. 14 illustrates schematically a design of the present study of CD8+ OT-I, T cell expansion with prime and boost immunizations of mHS-110 with different doses of mPTX-35. FIGS. 15-20 illustrate results of the study of FIG. 14. FIG. 17 illustrates percentage of CD8+ OT-I⁺ T-cells on days 4 and 38. Results of measurement of tumor size (over time) and weight (at the end of the study on day 52) are shown in FIGS. 18 and 19, respectively.

T cell receptor (TCR) transgenic mouse CD8+ (OT-I) cells were isolated from OT-I-GFP (eGFP-OT-I) mice using Easy Sep Mouse CD8⁺ T Cell isolation kit (STEMCELL Technologies, cat #19853A) and injected into each C57BL/6 mouse intravenously (i.v.) through lateral tail vein, with 1 million OT-I cells suspended in HBSS (GIBCO 14175-095). Two days after injecting OT-I cells, all the mice were tail bled for baseline, and 4 hours later, mHS-110 (B16F10-OVA-gp96-Ig) cells were treated with 10 μg/mL of Mitomycin-C(Sigma-Aldrich cat #M0503) for 2.5 hours and given intraperitoneally (i.p.) to each group accordingly. Mice were divided into five groups, with 5 mice per group. Animals were dosed based on nanogram expression level (per 10⁶ cells per 24 hr) of gp96-Ig, with the study design shown in FIG. 14.

Mice were tail bled consecutively on days 0, 2, 4, 6, 8, 10, 12, and 14 post-immunization and days 16, 18, 20, 22, 24, 26, 28, 35, 38, 41, 45, 49, and 52 into heparinized PBS (10 units/ml) and lysed using ACK lysis buffer (150 mM NH₄Cl, 100 mM KHCO₃ and 10 mM EDTA 0.2 Na, pH 7.2) for 3 minutes and neutralized with 1×PBS. To distinguish the OT-I transferred cells, samples were centrifuged at 300×g for 5 minutes, supernatant was removed and the cell pellet stained with anti-CD3 (20 μg/ml), anti-CD44 (20 μg/ml), anti-KLRG1 (20 μg/ml) and anti-CD8 (5 μg/ml) antibody cocktail made in FACS buffer using Alexa Fluor 700 anti-mouse CD3 (BioLegend, cat #100216), PE-Cy7 anti-mouse CD44 antibody (BioLegend cat #103030), APC anti-mouse KLRG1 (BioLegend cat #138412) and Brilliant Violet 421 anti-mouse CD8alpha antibody (BioLegend cat #100738) for 30 minutes at 4° C.

Melanoma B16F10 cells were harvested and resuspended at a concentration of 5×10⁵ cells/100 μl in a volume of 80 μl HBSS and 20 μl Matrigel. C57BL/6 mice were subcutaneously injected with 100 μl of B16F10 cells (5×10⁵ cells/mouse) on the inner abdomen 29 days post-OT-I transfer and 28 days post-primary vaccination, designated “d28”, as shown in FIG. 14. The tumor size was measured and documented every 2 days with a caliper, starting on day 5, and calculated using the formula (A×B) (A as the largest and B as the smallest diameter of tumor). Tumor growth was documented as standard error mean. To record the survival of the tumor-bearing mice, either natural death or a tumor volume greater than 450 mm² leading to death was counted as death. Each experimental group included five animals.

The dose of gp96-Ig (mHS-110) of 100 ng, released in a 24-hour period per million cells in 1 mL of medium, was defined as sub-optimal. Thus, the study of FIG. 14 was designed with doses of gp96-Ig that would produce enough T cell stimulation to detect any synergistic activity that the addition of a TNFRSF25 agonist (e.g., mPTX-35) may produce. In particular, several doses of mPTX-35 were provided in the following groups (shown in FIG. 14): 100 ng mHS-110, 100 ng mHS-110+0.1 mg/kg mPTX-35, 100 ng mHS-110+1 mg/kg mPTX-35, 100 ng mHS-110+10 mg/kg mPTX-35, and 10 mg/kg mPTX-35. At the end of study, GFP CD3+CD8+CD44+blood, spleen, and tumor (tumor-infiltrating lymphocytes (TILs)) were measured, as well as memory marker KLRG1.

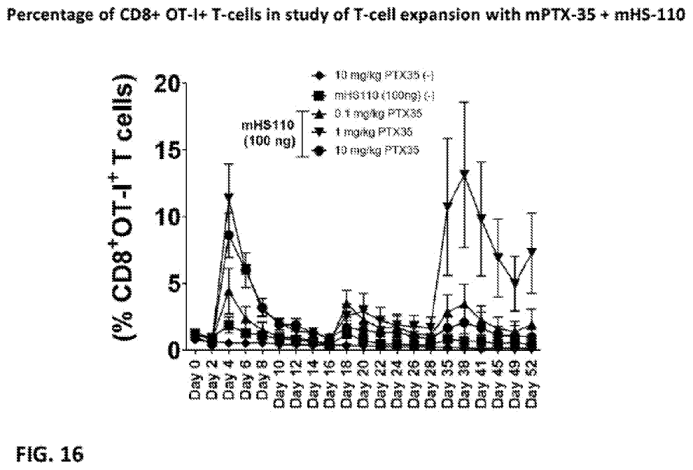

FIG. 15 shows flow cytometry gating strategy (day 4) and cellular expansion of CD8⁺ OT-I cells, with mPTX-35+mHS-110 in the following analyzed groups: 10 mg/kg mPTX-35 (panel 1), 100 ng mHS-110 (panel B), 100 ng mHS-110+0.1 mg/kg mPTX-35 (panel C), 100 ng mHS-110+1 mg/kg mPTX-35 (panel D), and 100 ng mHS-110+10 mg/kg mPTX-35 (panel E). The data in FIG. 15 illustrates synergistic expansion of CD8⁺ OT-I cells with mPTX-35+mHS-110, which is particularly prominent for 100 ng mHS-110+1 mg/kg mPTX-35 (panel D) and 100 ng mHS-110+10 mg/kg mPTX-35 (panel E). FIG. 16 shows cell expansion, as a percentage of CD8⁺ OT-I⁺ T cells, over time (0-52 days), in the same groups as shown in FIGS. 14 and 15. FIG. 17 shows percentage of CD8⁺ OT-I⁺ T cells on day 4 (panel A), and percentage of CD8⁺ OT-I⁺ T cells on day 38 (panel B). Graphs show mean+SEM, *p<0.05; **p<0.01 by Mann-Whitney, and “ns” indicates “not significant.” As illustrated in FIGS. 16 and 17, the TNFRSF25 agonist, mPTX-35, synergizes with a gp96-Ig-secreting cancer vaccine, mHS-110, to expand anti-tumor effector T cells. Specifically, on days 4 and 38, 100 ng mHS-110+1 mg/kg mPTX-35 and 100 ng mHS-110+10 mg/kg mPTX-35 treatment groups exhibit the prominent increase in the percentage of CD8+ OT-I⁺ T cells, as compared to 10 mg/kg mPTX-35 and 100 ng mHS-110 treatment groups.

Combination of mPTX-35 with mHS-110 increased the expansion of OVA-specific CD8⁺ T-cells, in an mPTX-35 dose dependent manner (FIG. 15), with the greatest expansion observed after the primary immunization and second boost, post-tumor implantation (FIGS. 16 and 17), on days 4 and 38, respectively. As shown in FIGS. 16 and 17, the cellular expansion was greatest for the 1 mg/kg dose of mPTX-35 with mHS-110 and greatly exceeded the additive value of mPTX-35 and mHS-110 treatment alone, strongly demonstrating that mPTX-35 synergizes with mHS-110 to expand antigen specific CD8⁺ T-cells.

FIG. 18 illustrates synergistic antigen-specific T cell expansion and tumor cell killing with mPTX-35 as tumor growth kinetics for the five groups studied in this example in a murine model. As shown in FIG. 18, a combination of mPTX-35 with mHS-110 delays tumor growth to a larger degree than mPTX-35 alone. Accordingly, as shown in FIGS. 15-18, using adoptively transferred, TCR-transgenic, OVA-antigen specific CD8⁺ T cells (OT-I) mHS-110 vaccination with mPTX-35 showed that TNFRSF25 engagement in the presence of antigen synergizes to rapidly expand antigen-experienced CD8⁺ T cells that exhibit enhanced recall and longevity. FIG. 19 shows tumor weight bar graphs (panel A) and tumor weight scatter plots (panel B) illustrating the end tumor mass (day 52) for the five studied groups, which also demonstrate that the combination of mPTX-35 with mHS-110 delays tumor growth.

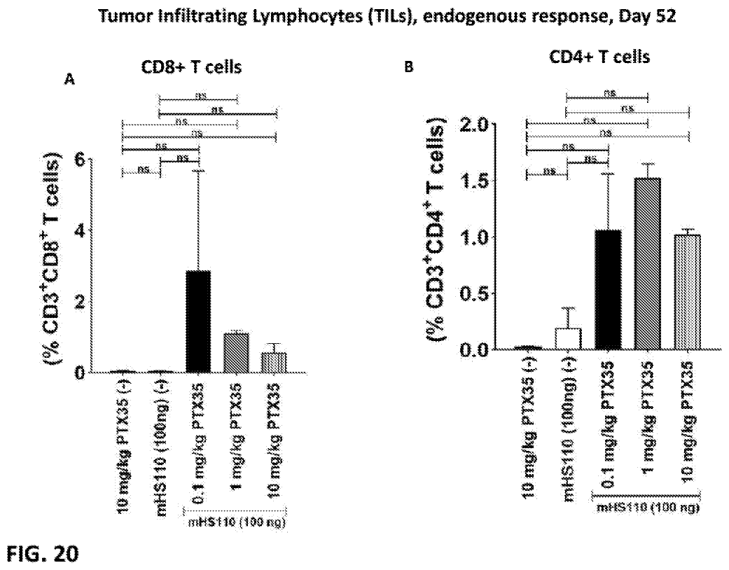

The use of a therapeutic model as in the present example, where tumors can grow, prior to immunization, allows for a greater therapeutic window and thus allows to evaluate the effect of a combination with mPTX-35. In the study of FIG. 14, from the tumors that remained on day 52 (see FIG. 16), from non-deceased animals, tumor infiltrating leukocytes (TILs) were extracted and analyzed by flow cytometry. Total CD4+ and CD8⁺ T cells, by percentage, were analyzed in the TILs of digested tumors. The MACS Miltenyl Biotec tumor dissociation kit was used for tumor digestion procedure (cat #130-096-730). The results of the analysis of TILs are shown in panel A (CD8⁺ T cells) and panel B (CD4⁺ T cells) of FIG. 20. As shown in FIG. 20, the number of CD4+ and CD8+TILs increases when mPTX-35 is administered along with mHS-110.

Results in FIGS. 15-20 demonstrate in vivo synergistic activity of the combination of TNFRSF25 agonist, mPTX-35, with the gp96-Ig secreting, cancer vaccine mHS-110 (e.g., suboptimal doses), to rapidly expand anti-tumor CD8⁺ T cells, and inhibit tumor growth. The most effective dose of mPTX-35 used in combination with 100 ng of gp96-Ig was 1 mg/kg, administered simultaneously with vaccination every two weeks, i.p. The results also demonstrate that DR3-agonism in the context of antigen challenge enhances T-cell expansion, using mPTX-35 rather than 4C12.

### Example 6: Study of Synergy of PTX-35 with Gp96-Ig (mHS-110)/OX40L-Ig (mHS-130

The objective of this study was to compare the effect of mHS-110, mHS-130, and mPTX-35, alone or in a combination, on expansion of OT-1 CD8⁺ T cells, and to evaluate the impact on tumor growth delay.

In this example, B16F10-OVA and OT-1 challenge model was used, along with the gp96-Ig expressing B16F10-OVA expressing cell line (mHS-110), and along with the OX40L-Ig (mHS-130) expressing cell line.

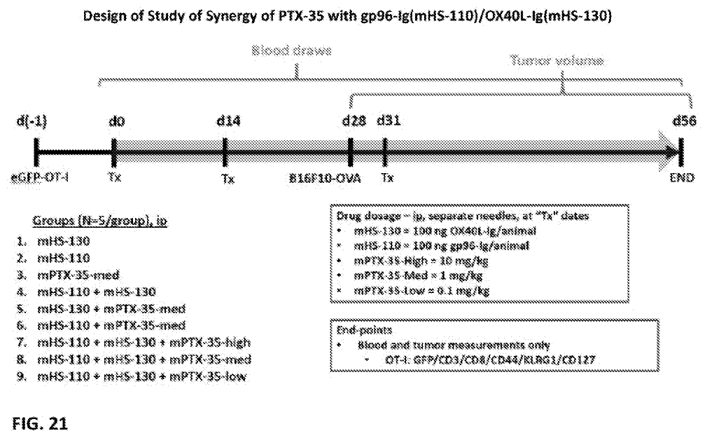

FIG. 21 illustrates the design of the synergistic activity of mPTX-35 with gp96-Ig (mHS-110) and OX40L-Ig (mHS-130), in vivo, for both expansion of CD8⁺ T-cells as well as delayed outgrowth of implanted syngeneic tumors. The study included the following groups (n=5/group): (1) mHS-130/OX40L-Ig (100 ng), (2) mHS-110/gp96-Ig (100 ng), (3) mPTX-35 (1 mg/kg), (4) mHS-110+mHS-130, (5) mHS-110+mPTX-35 (1 mg/kg), (6) mHS-130+mPTX-35 (1 mg/kg), (7) mHS-110+mHS-130+mPTX-35 (10 mg/kg), (8) mHS-110+mHS-130+mPTX-35 (1 mg/kg), and (9) mHS-110+mHS-130+mPTX-35 (0.1 mg/kg). The doses of gp96-Ig (mHS-110) and OX40L-Ig (mHS-130) were 100 ng per animal. The doses of mPTX35 were “mPTX35-Low” (0.1 mg/kg), “mPTX35-Med” (1 mg/kg), and “mPTX35-High” (10 mg/kg).

Cell Line Protein Expression Data for mHS-110 and mHS-130

The amount of murine gp96 protein expressed by the mHS-110 cells was determined by ELISA. For each sample to be tested, one million B16F10-Ova9 parental and mHS-110 cells were plated in a 6-well tissue culture plate in a total volume of 1 ml each. Cells were incubated at 37° C. with 5% CO₂ for 24 hours, at which point the supernatants were harvested. The supernatants were then centrifuged at 2500 rpm for 5 minutes to pellet any cell debris. Clarified supernatants were then transferred to new 1.5 ml tubes and stored at −80° C. Each sample tested was from a fresh vial of mHS-110 cells thawed and expanded.

To perform the ELISA, 96-well plates (Corning, cat #9018) were coated with 2 ug/ml of sheep anti-gp96 (R&D Systems, cat #AF7606) in carbonate-bicarbonate buffer. Plates were sealed and stored at 4° C. overnight. Plates were then washed 4 times with 1×TBST (VWR, cat #K873) and then blocked with 1× casein solution (Sigma, cat #B6429) for 1 hour at room temperature. The plates were then washed 4 times with 1×TBST and a human gp96-mouse Fc standard (Thermo, lot #2065447) was prepared in IMDM (Gibco, cat #12440-053) with 10% FBS (Gibco, cat #10082-147). A 1000 ng/ml human-gp96-mFc standard solution was made and 2-fold serial dilutions were performed down to 0.977 ng/ml. Sample supernatants were loaded onto the ELISA plates starting at a 1:2 dilution and then 2-fold serial dilutions were performed to a highest dilution of 1:16. Plates were sealed and incubated for 1 hour at room temperature and then washed 4 times with 1×TBST. The detection antibody, goat anti-mouse IgG (Fc)-HRP (Jackson ImmunoResearch Laboratories, Inc., cat #115-036-008) was diluted 1:5,000 (80 ng/ml) in 1×TBST and added to the ELISA plates. Plates were then sealed and incubated in the dark for 1 hour at room temperature. After washing the plates 4 times with 1×TBST, TMB substrate (SeraCare, cat #5120-0076) was added to each well and incubated in dark for 15 minutes at room temperature. Reactions were then stopped with 1N sulfuric acid and plates read on the Biotek ELx800 plate reader. Concentrations of gp96 expressed from each sample were then determined based off the standard curve. This protocol was based on the human gp96-Ig protocol. For this study, the following protein levels were provided as shown in Table 1 below:

TABLE 1
Expression of mouse gp96-Ig from mHS-110 per
million cells in a 24-hour period
Mouse gp96-Ig (nanograms/mL per million cells in 24 hrs)

| | | | | Average |
|---|---|---|---|---|
| Repli- | Prime Dose: | 1^{st} Boost Dose: | 2^{nd} Boost Dose: | Expres- |
| cate | Concentration | Concentration | Concentration | sion |
| 1 | 127.65 | 72.50 | 91.90 | 97.35 |
| 2 | 129.07 | 71.71 | 85.04 | 100.39 |
| 3 | 127.65 | 74.07 | 92.67 | 100.86 |
| 4 | 131.45 | 65.08 | 96.52 | 97.68 |
| 5 | 125.12 | 74.10 | 66.97 | 88.73 |
| 6 | 126.38 | 68.08 | 90.59 | 95.02 |
| Mean | 127.89 | 70.92 | 87.28 | 99.41 |
| STDEV | 2.20 | 3.61 | 10.62 | 5.48 |
| CV % | 1.72 | 5.10 | 12.17 | 6.33 |

The amount of mouse OX40L protein expressed by the mHS-130 cells was also determined by ELISA. For each sample to be tested, one million B16F10-Ova9 parental and mHS-130 cells were plated in a 6-well tissue culture plate in a total volume of 1 ml each. Cells were incubated at 37° C. with 5% CO₂ for 24 hours at which point the supernatants were harvested. Supernatants were then centrifuged at 2500 rpm for 5 minutes to pellet any cell debris. Clarified supernatants were then transferred to new 1.5 ml tubes and stored at −80° C. Samples collected were from freshly thawed vials of cells.

To perform the ELISA, 96-well plates were coated with 2.5 ug/ml His-tagged mouse OX40 protein (Acro Biosystems, cat #OXO-M5228) in PBS. Plates were sealed and stored at 4° C. overnight. Plates were then washed 4 times with 1×TBST and then blocked with 1% BSA (Sigma, cat #A2153) for 1 hour at room temperature. The plates were then washed 4 times with 1×TBST and a mouse IgG1-mouse OX40L standard (Thermo, lot #2214217) was prepared in IMDM with 10% FBS. A 2000 ng/ml mIgG1-mOX40L standard solution was made and 2-fold serial dilutions were performed down to 1.95 ng/ml. Sample supernatants were loaded onto the ELISA plates and 2-fold serial dilutions were performed. Plates were sealed and incubated for 90 minutes at 37° C. and then washed 4 times with 1×TBST. The detection antibody, goat anti-mouse IgG (Fc)-HRP (Jackson ImmunoResearch Laboratories, Inc., cat #115-036-008), was diluted 1:5000 in 1×PBS/0.05% Tween 20/0.1% BSA and added to the ELISA plates. Plates were then sealed and incubated in the dark for 1 hour at room temperature. After washing the plates 4 times with 1×TBST, TMB substrate was added to each well and incubated in dark for 10 minutes at room temperature. Reactions were then stopped with 1N sulfuric acid and plates read on the Biotek ELx800 plate reader. Concentrations of OX40L expressed from each sample were then determined based off the standard curve. This protocol is based on human OX40L protocol. For this study, the following protein levels were provided as shown in Table 2 below:

TABLE 2
Expression of mouse OX40L-Ig from mHS-130 per
million cells in a 24-hour period
Mouse OX40L-Ig (nanograms/mL per million cells in 24 hrs)

| | | | | Average |
|---|---|---|---|---|
| Repli- | Prime Dose: | 1^{st} Boost Dose: | 2^{nd} Boost Dose: | Expres- |
| cate | Concentration | Concentration | Concentration | sion |
| 1 | 325.90 | 492.77 | 339.78 | 386.15 |
| 2 | 366.85 | 487.84 | 371.56 | 427.35 |
| 3 | 374.56 | 423.26 | 378.75 | 398.91 |
| 4 | 372.89 | 590.55 | 416.36 | 481.72 |
| 5 | 392.79 | 475.08 | 402.53 | 423.47 |
| 6 | 415.34 | 452.81 | 408.43 | 425.53 |
| Mean | 374.72 | 487.05 | 386.24 | 416.00 |
| STDEV | 29.75 | 56.80 | 28.62 | 38.39 |
| CV % | 7.94 | 11.66 | 7.41 | 9.00 |

OT-1 Purification, Adoptive T-Cell Transfer, mHS-110/mPTX-35 Dosing, and Flow Cytometry Staining

As shown in FIG. 21, T cell receptor (TCR) transgenic mouse CD8+ (OT-I) cells were isolated from in-house bred OT-I-GFP mice using Easy Sep Mouse CD8 T Cell isolation kit (cat #19853A) and injected into each C57BL/6 mouse intravenously (i.v) through retro-orbital vein with 1 million OT-I cells suspended in HBSS (GIBCO 14175-095). Two days after injecting OT-I cells, all the mice were tail bled for baseline and 4 hours later, mHS-110 (B16F10-OVA-gp96-Ig) cells were treated with 10 μg/mL of Mitomycin-C(Sigma-Aldrich cat #M0503) for 3 hours and given intraperitoneally (i.p.) to each group accordingly along with different doses of mPTX35 (“mPTX35-Low” or “mPTX35low” (0.1 mg/kg), “mPTX35-Med” or “mPTX35med” (1 mg/kg), and “mPTX35-High” or “mPTX35hi” (10 mg/kg)). Mice were divided into 9 groups with 5 mice per group, and the animals were dosed based on nanogram expression level (per 10⁶ cells per 24 hr) of gp96-Ig and OX40L-Ig per Tables 1 and 2 and the study design shown in FIG. 21.

Mice were tail bled consecutively on days 0, 3, 5, 7, 10, 12, and 14 post-immunization and days 17, 19, 21, 24, 26, 28, 31, 34, 38, 41, and 46 into heparinized PBS (10 units/ml) and lysed using ACK lysis buffer (150 mM NH₄Cl, 100 mM KHCO3 and 10 mM EDTA 0.2 Na, pH 7.2) for 2 minutes and neutralized with 1×PBS. To distinguish OT-I transferred cells, samples were centrifuged at 300×g for 5 minutes, supernatant was removed and the cell pellet stained with anti-CD3 (20 μg/ml), anti-CD44 (20 μg/ml), anti-CD127 (20 μg/ml), anti-KLRG1 (20 μg/ml) and anti-CD8 (5 μg/ml) antibody cocktail made in FACS buffer using Alexa Fluor 700 anti-mouse CD3 (BioLegend cat #100216), PE-Cy7 anti-mouse CD44 antibody (BioLegend Cat #103030), PE anti-mouse CD127 (BioLegend cat #135010), APC anti-mouse KLRG1 (BioLegend Cat #138412) and Brilliant Violet 421 anti-mouse CD8alpha antibody (BioLegend Cat #100738) for 30 minutes at 4° C. All samples were centrifuged and washed with FACS buffer after staining and 300 μl of FACS buffer was added and 30,000 CD3 gated events were collected on the Sony Flow Cytometer (SH-800).

B16F10-OVA Tumor Challenge and Volume Calculations

Melanoma B16F10 cells were harvested and resuspended at a concentration of 5×10⁵ cells/100 μl in a volume of 80 μl HBSS and 20 μl Matrigel. C57BL/6 mice were subcutaneously injected with 100 μl of B16F10 cells (5×10⁵ cells/mouse) on the inner abdomen 29 days post-OT-I transfer and 28 days post-primary vaccination, designated “day 28”, as shown in FIG. 21. The tumor size was measured and documented every 2 days with a caliper, starting on day 5, and calculated using the formula (A×B) (A as the largest and B as the smallest diameter of tumor). Tumor growth was documented as standard error mean. To record the survival of the tumor-bearing mice, either natural death or a tumor volume greater than 450 mm² leading to death was counted as death. Each experimental group included five animals.

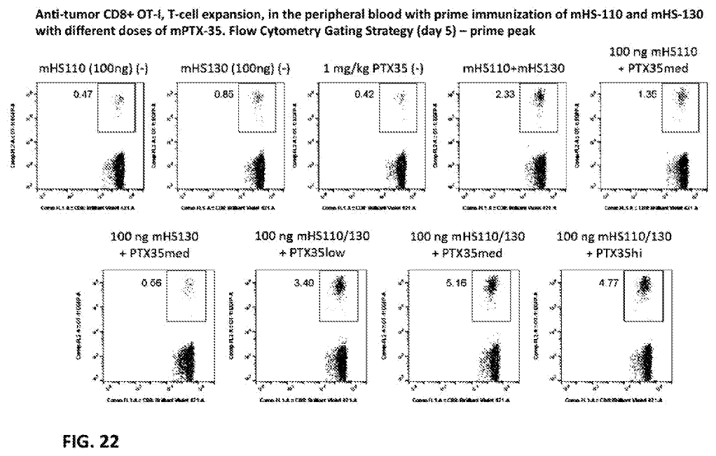

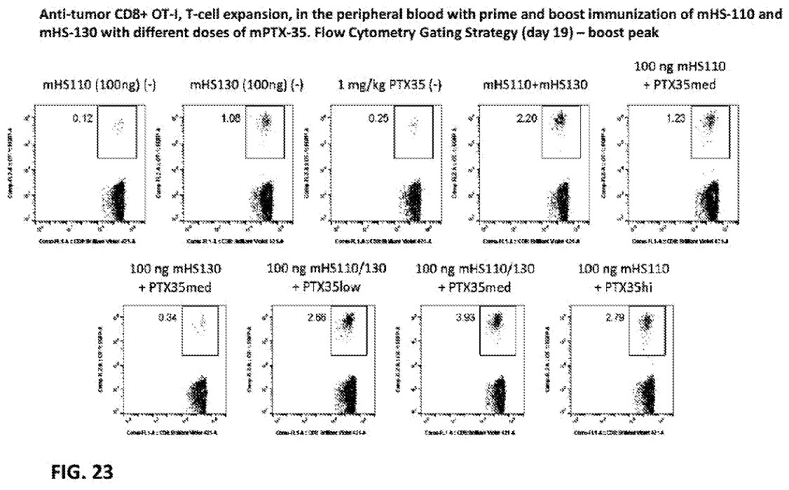

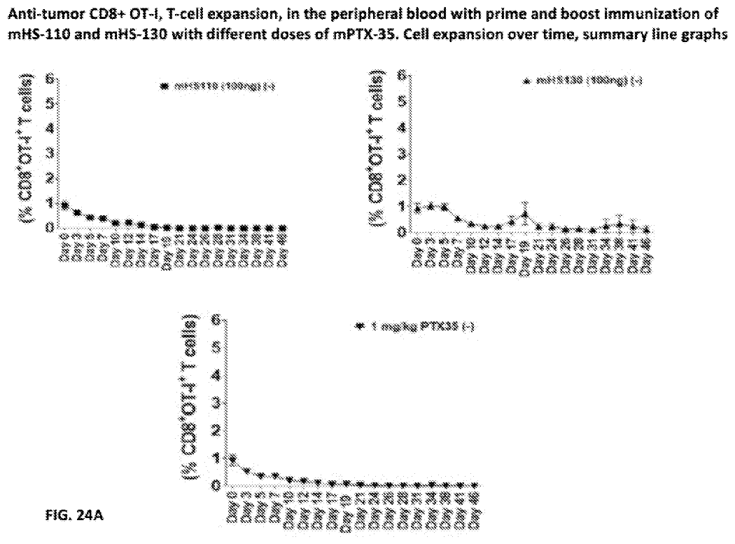

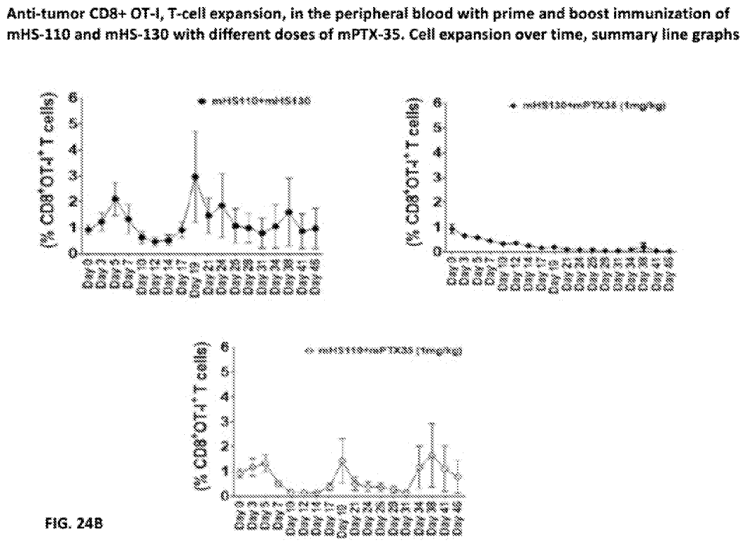

FIGS. 22 to 31 illustrate anti-tumor CD8+ OT-I, T-cell expansion, in the peripheral blood with prime and boost immunization of mHS-110 and mHS-130 with different doses of mPTX-35. FIG. 22 shows flow cytometry gating strategy (day 5)—prime peak, FIG. 23 shows flow cytometry gating strategy (day 19, boost peak), and FIGS. 24A, 24B, and 24C illustrate cell expansion over time as the summary line graph for OT-1 cells. As shown in FIGS. 22, 23, and 24A-24C, combination of mPTX-35 with mHS-110 and mHS-130 (which are, in this example, separate cell lines secreting gp96-Ig and OX40L-Ig, respectively) led to an mPTX-35 increase in the primary immune response and secondary boost peaking at days 5 and 19, respectively. At the doses of mHS-110 and mHS-130 used in this example (100 ng of gp96-Ig and OX40L-Ig, respectively), both vaccines alone were not enough to expand OT-1 cells in mice. The same was observed for the dose of 1 mg/kg of mPTX-35. Combination of mHS-110 and mHS-130 provided the prime/boost response. Combining mHS-130 (OX40L-Ig) with mPTX-35 at 1 mg/kg did not expand OT-1 cells; however, combination with mHS-110 did, as shown in FIGS. 24B and 24C. It was observed, based on the internal quality control ELISAs, that the expression of OVA in the mHS-130 line is lower than that of the mHS-110 (about 10× lower, data not shown). However, the expression of OX40L-Ig and the expression of gp96-Ig were measured, and were both provided at the same level of 100 ng. Thus, it was observed that the combination of an anti-TNFRSF25 agonist mAb and an OX40-stimulating ligand does not lead to the expansion of anti-tumor CD8⁺ T cells, but the introduction of gp96-Ig does. This suggests, without wishing to be bound by the theory, that gp96-Ig provides a necessary stimulation step that helps expand CD8⁺ T-cells over that of other molecules.

The greatest expansion of CD8+ OT-1⁺ T cells was observed in the peripheral blood when mPTX-35 was combined with both mHS-110 and mHS-130, as shown in FIGS. 22-30. This expansion was observed for both the primary and secondary challenge and was greatest for the 1 mg/kg dose of mPTX-35, consistent with the studies described above where 1 mg/kg of the TNFRSF25 agonist mAb gave the most prominent response against tumor growth. At the same time, 0.1 mg/kg of mPTX-35 was enough to produce a lasting effect that produced a similar curve and about 60% of the area under the curve (AUC) of both 1 mg/kg and 10 mg/kg of mPTX-35, as shown in FIG. 24C, suggesting that the appropriate dose of mPTX-35 in combination with OX40L-Ig and gp96-Ig is between 0.1 mg/kg and 1 mg/kg. The second immunization boost also produced the induction of OT-I T-cells, though less pronounced than the first and second vaccination. It is interesting to note, however, that this tertiary expansion of OT-1 cells was greatest with mHS-110 and mPTX-35 alone, minus mHS-130, as shown in FIGS. 24B and 24C. This may suggest, without wishing to be bound by the theory, that there is interaction of OX40L-Ig altering the kinetics of T-cell expansion in the presence of TNFRSF25 agonism and OX40L.

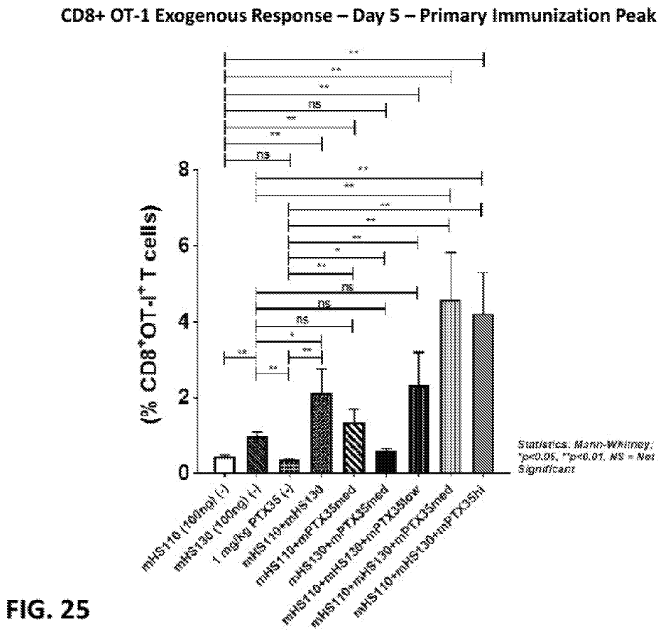

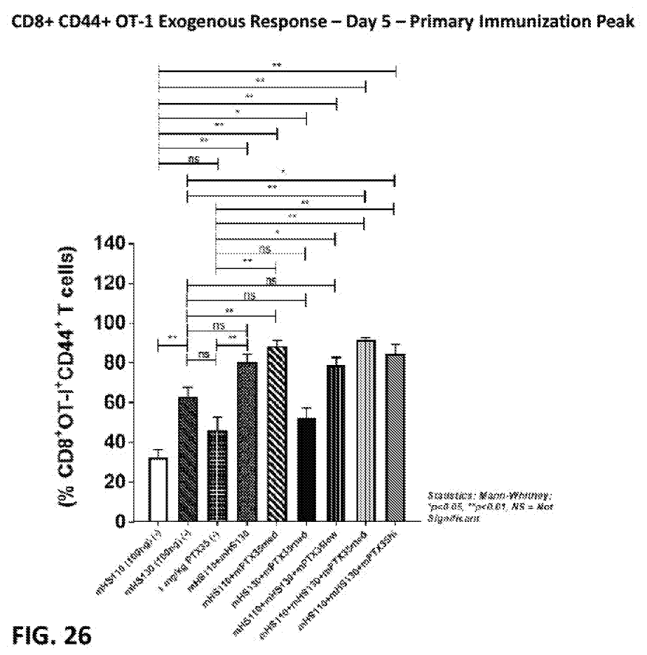

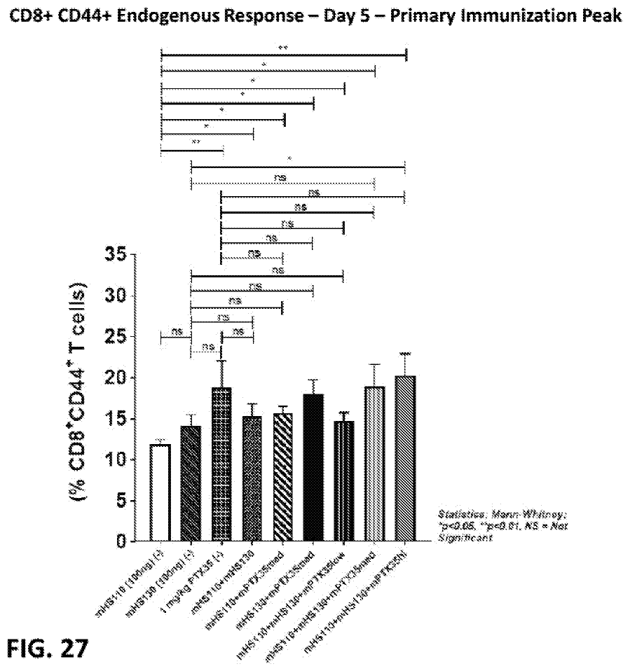

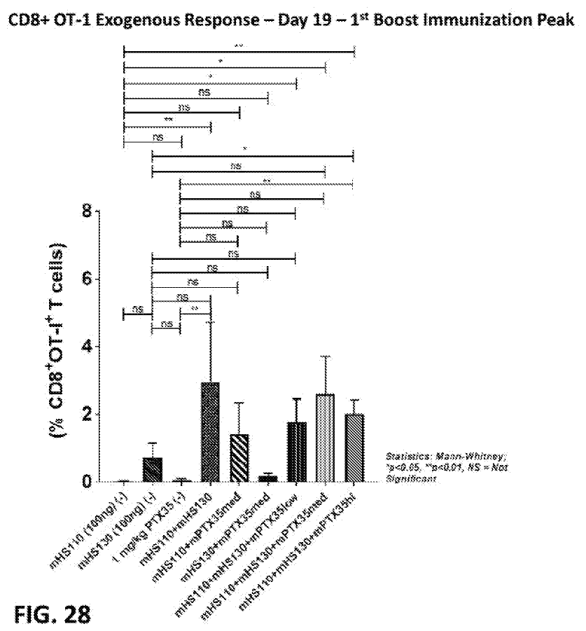

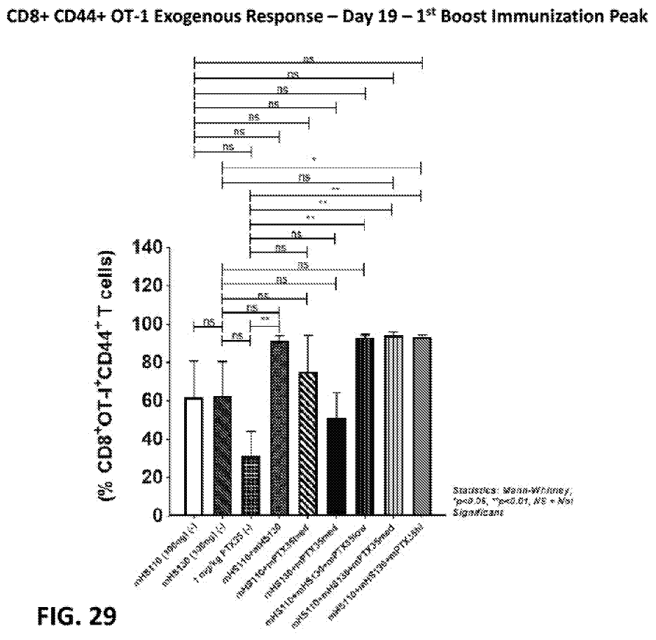

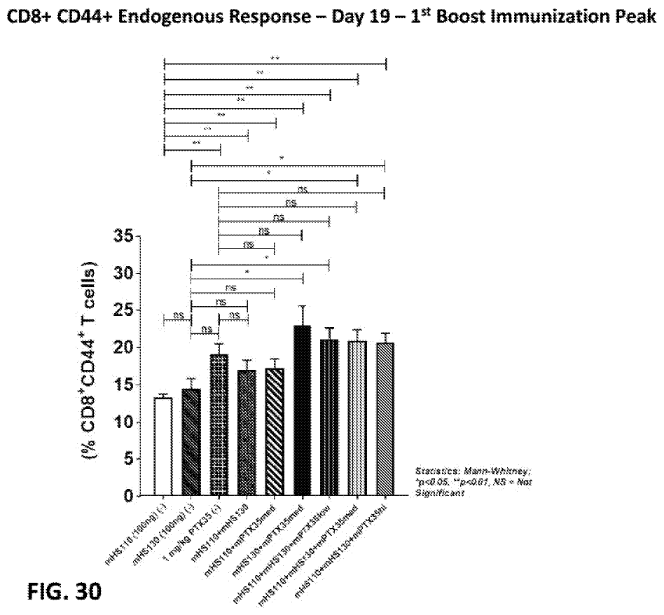

At peak OT-1 expansion in the blood of immunized mice, the triple treatment combinations (mHS-110+mHS-130+mPTX35low, mHS-110+mHS-130+mPTX35med, and mHS-110+mHS-130+mPTX35hi) all significantly increased OT-I expansion, as shown in FIG. 25 illustrating percent of gated CD8+ OT-1⁺ T cells from peripheral blood on day 5 after the primary immunization. It was discovered, surprisingly, that at the 1 mg/kg dose of mPTX-35 in combination with mHS-110/mHS-130, this combination synergistically enhanced cellular expansion of CD8⁺ T-cells in vivo. The percent of effector OT-1 cells alone, as shown in FIG. 26, evaluated for mPTX-35 in combination with mHS-110 alone was comparable to the triple combinations (mPTX-35+mHS-110+mHS-130), which suggests that OX40L-Ig may not be needed to induce effector T cells when both gp96 and TNFRSF25 ligation are present. When considering the percent of gated CD8⁺CD44⁺ endogenous T-cells on day 5, polyclonal responses mask individual contributing effects, but the dose responses of mPTX-35 in combination with mHS-110/130 are still obvious and significant over that of mHS-110 or mHS-130 alone (FIG. 27). With cellular expansion peaking on day 19, after the second immunization (secondary immune response), all three groups containing triple combinations of mPTX-35, mHS-110 and mHS-130 continue to show greater than 1% of all CD3⁺ T-cells in the peripheral blood specific for tumor antigen, as shown in FIG. 28. FIG. 28 also shows that the groups treated with the double combination of mHS-110 plus mHS-130 and with the double combination mHS-110 plus mPTX-35 also demonstrate greater than 1% of all CD3⁺ T-cells in the peripheral blood specific for tumor antigen. This trend continues with effector OT-1 cells for all three triple combinations and double combinations with mHS-110/130 and mPTX-35, as shown in FIG. 29. Endogenous, total effector CD8⁺ T-cells show a similar response, however less distinct, since polyclonal in nature, the combination of mPTX-35 with vaccination does in fact drive the expansion of effector CD8⁺ T cells above single agent controls alone (FIG. 30).

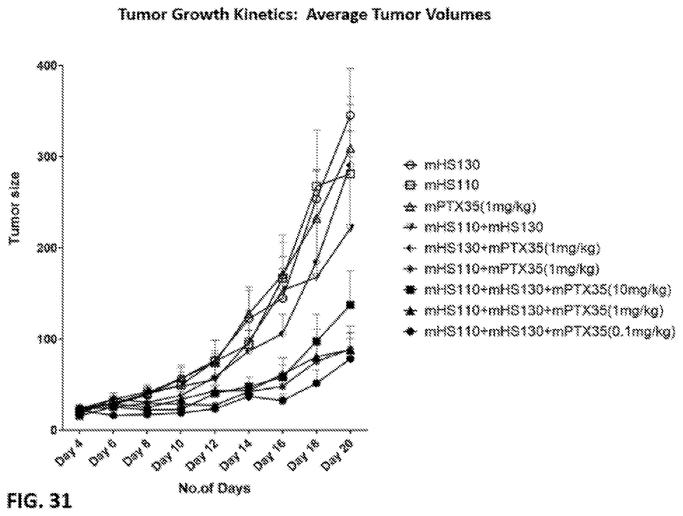

As shown in FIG. 21, on day 28 of the study, 14 days after the first boost immunization, mice were challenged with 500,000 B16F10-OVA expressing tumor cells, and tumor delay was monitored over time. As shown in FIG. 31 illustrating average tumor volumes on day 4 (study day 32) through day 20 (study day 48), all single-agent controls succumbed to tumor growth by day 20 post-initial tumor transplant. The double combination of mHS-110 with mHS-130 showed some control. As demonstrated with CD8⁺ T cell expansion, the addition of gp96-Ig to TNFRSF25 agonism shows a better response for tumor growth inhibition than that of OX40L-Ig. When all three treatments were combined, 0.1 mg/kg, 1 mg/kg, and 10 mg/kg of mPTX-35 were able to inhibit the outgrowth of tumor, as shown in FIG. 31.

Thus, the results of the study in the present example (see FIG. 21), as shown in FIGS. 22-31, demonstrate that adding gp96-Ig (mHS-110) to mPTX-35 (TNFRSF25 agonism) greatly enhances anti-tumor immunity, with the effect being greater than the effect of the addition of OX40L-Ig (mHS-130) to mPTX-35. However, the combination of all three (mHS-110+mHS-130+mPTX-35) expands T cells to the greater degree and can protect against tumor outgrowth more effectively, as compared to the effects of double combinations (vaccine plus mPTX-35) alone. Also, upon the second boost (third immunization) with mHS-110 and mPTX-35, but not mHS-130, CD8⁺ T cells show a characteristic surge expansion not observed with the triple or single treatments. Using the B16F10-OVA and OT-I challenge model along with the gp96-Ig expressing B16F10-OVA expressing cell line (mHS-110), and another expressing OX40L-Ig (mHS-130), this study demonstrates the first synergistic activity in vivo of mPTX-35 with the 100 ng doses of gp96-Ig and OX40L-Ig, for both expansion of CD8⁺ T cells as well as delayed outgrowth of implanted syngeneic tumors. Accordingly, this study shows that the combination of two costimulation agonist molecules, specifically targeting OX40 and TNFRSF25, can synergize with gp96-Ig cancer immunotherapy to prevent the outgrowth of aggressive tumor types and greater expand antigen specific CD8⁺ T cells.

### Example 7: A Further Study of Synergy of PTX-35 with Gp96-Ig (mHS-110)/OX40L-Ig (mHS-130

The objective of this study was to assess the role of mHS-110 and mHS-130 in combination with an agonist TNFRSF25 monoclonal antibody (mAb), PTX-35.

In this example, to study expansion, contraction, and maintenance of tumor-specific CD8+ T cell responses, mHS-110 and/or mHS-130, in combination with different doses of mouse-IgG1-PTX-35 (mPTX-35) were administered to C57BL/6 mice that were adoptively transferred with syngeneic OVA-specific T cells (OT-I). Mice were then challenged with murine melanoma tumors (B16F10-OVA) to characterize the tumor-specific immune cells in the periphery, spleen, and tumor-microenvironment that were involved in tumor regression. Combination of mPTX-35 with mHS-110 and mHS-130 increased the expansion of tumor-specific CD8+ T-cells, in an mPTX-35 dose-dependent manner. This cellular expansion was significantly higher in the 1 mg/kg dose of mPTX-35 and far exceeded the additive value of mPTX-35, mHS-130, and mHS-110 treatment alone. Systemic administration of mPTX35, in combination with mHS-110 and mHS-130, led to a significant increase in the expansion of activated CD8⁺ T cells in the blood and stimulated activation of effector memory CD8 T cells residing in the spleen. Importantly, this combination resulted in higher frequencies of tumor infiltrating lymphocytes (TILs), which enhanced regression of established B16F10-OVA tumors and increased overall survival. These results strongly suggest that mPTX35 synergizes with mHS-110 and mHS-130 to amplify activated tumor-specific CD8⁺ T cells, program a strong memory response, and allow for tumor regression.

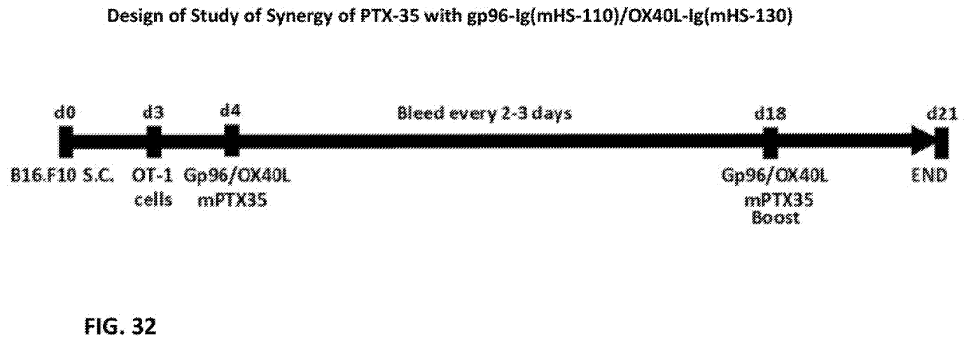

FIG. 32 illustrates a design of the study assessing the effect of combining PTX-35 with gp96-Ig/OX40L-Ig. C57BL/6 mice (n=5 mice per group) were injected subcutaneously (S.C.) with 500,000 B16.F10 melanoma tumors and 3 days later adoptively transferred with one-million syngeneic OT-I transgenic CD8 T cells. Mice were subsequently treated on days 4 (gp96-Ig/OX40L-Ig+mPTX35) and 18 (gp96-Ig/OX40L-Ig+mPTX35, boost) post tumor inoculation and monitored for tumor growth inhibition (TGI). The mice were sacrificed on day 21 post-tumor inoculation when the mice reached tumor burden. The study included the following six groups: (1) vehicle (PBS), (2) 1 mg/kg PTX-35 (−), (3) mHS-110+mHS-130 (0 PTX-35), (4) mHS-110+mHS-130+0.1 mg/kg PTX-35, (5) mHS-110+mHS-130+1 mg/kg PTX-35, (6) mHS-110+mHS-130+10 mg/kg PTX-35.

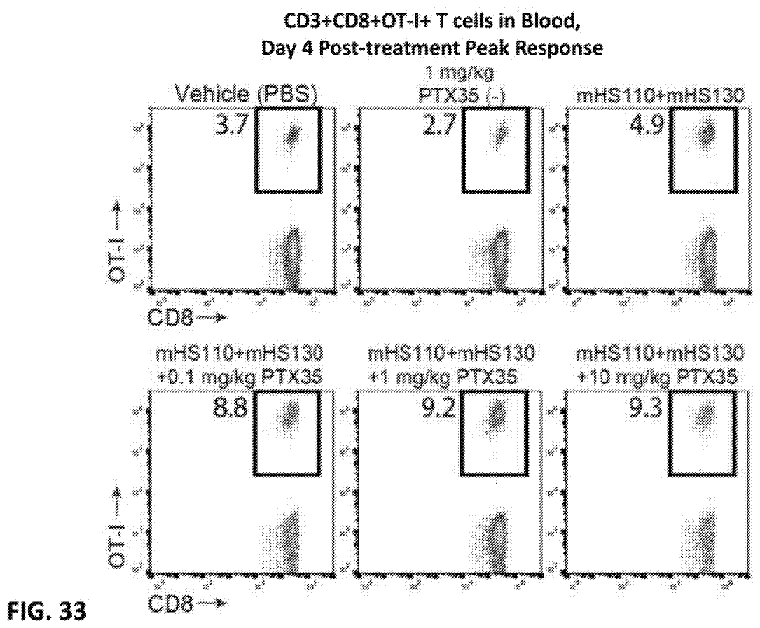

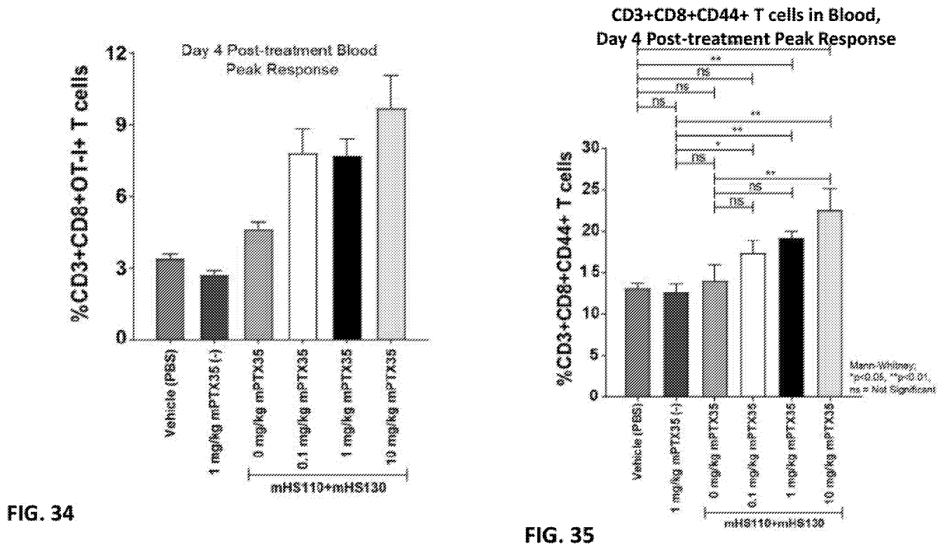

FIG. 33 shows a percentage of CD3+CD8+ OT-I⁺ T cells, as a representative FACS plot, that were enumerated in the peripheral blood during the peak of CD8 T cell responses on day 4 post-treatment in the following groups: (I) top panels: vehicle (PBS), 1 mg/kg PTX-35, mHS-110+mHS-130; and (II) bottom panels: mHS-110+mHS-130+0.1 mg/kg PTX-35, mHS-110+mHS-130+1 mg/kg PTX-35, and mHS-110+mHS-130+10 mg/kg PTX-35. FIG. 34 illustrates a percentage of CD3+CD8+ OT-I+ T cells during the peak of CD8 T cell responses on day 4 post-treatment in the same groups as in FIG. 33, as the mean±SEM.

Table 3 below shows statistics for percent of CD3+CD8+ OT-I+ T cells in blood, where the Mann-Whitney two-tailed test was used to determine statistical significance: *p<0.05, **p<0.01, “NS”=Not Significant.

TABLE 3
Statistics for percent of CD3+CD8+OT-I + T cells in blood.

| | | | | mHS-110 + | mHS-110 + | mHS-110 + |
|---|---|---|---|---|---|---|
| | Vehicle | 1 mg/kg | mHS-110 + | mHS-130 + 0.1 | mHS-130 + 1 | mHS-130 + 10 |
| Group | (PBS) | PTX-35 (−) | mHS-130 | mg/kg mPTX-35 | mg/kg mPTX-35 | mg/kg mPTX-35 |
| Vehicle (PBS) | — | NS | ** | ** | ** | ** |
| 1 mg/kg PTX-35 | NS | — | ** | ** | ** | ** |
| (−) | | | | | | |
| mHS-110 + | ** | ** | — | * | * | ** |
| mHS-130 | | | | | | |
| mHS-110 + | ** | ** | * | — | NS | NS |
| mHS-130 + 0.1 | | | | | | |
| mg/kg mPTX-35 | | | | | | |
| mHS-110 + | ** | ** | * | NS | — | NS |
| mHS-130 + 1 | | | | | | |
| mg/kg mPTX-35 | | | | | | |
| mHS-110 + | ** | ** | ** | NS | NS | — |
| mHS-130 + 10 | | | | | | |
| mg/kg mPTX-35 | | | | | | |

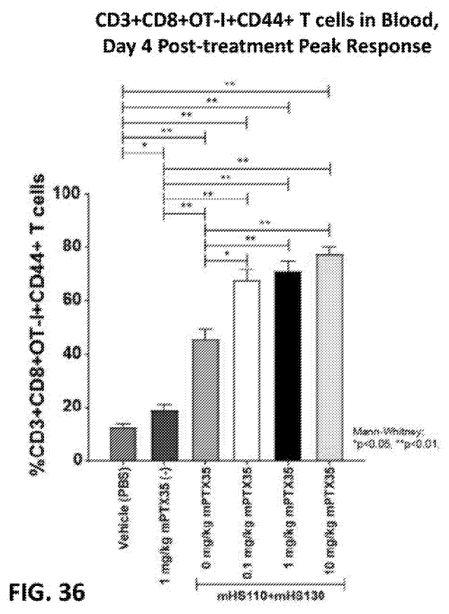

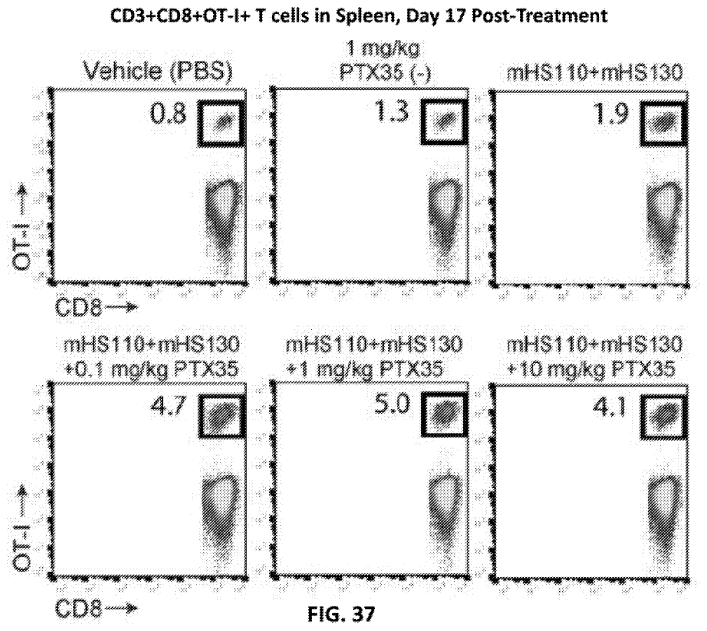

FIG. 35 shows a percentage of CD3+CD8+CD44+ T cells and FIG. 36 shows a percentage of CD3+CD8+ OT-I+CD44+ T cells, which were enumerated during the peak of CD8 T cell responses on day 4 post-treatment and are presented as the mean±SEM.

FIGS. 32 to 36 illustrate that PTX-35 synergizes with mHS-110/mHS-130 to enhance activated tumor-specific CD8 T cells.

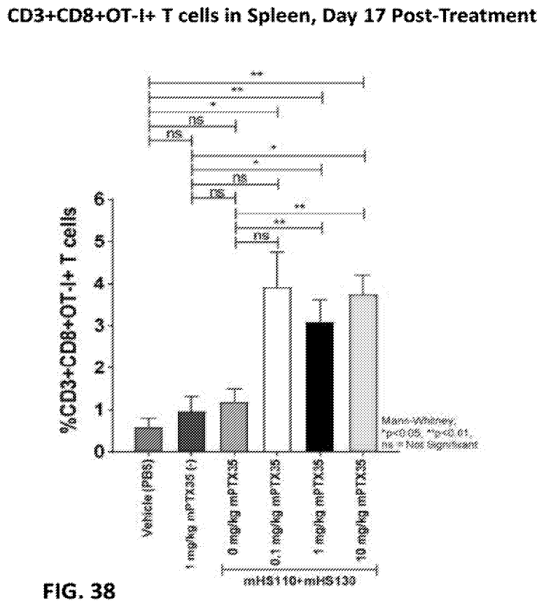

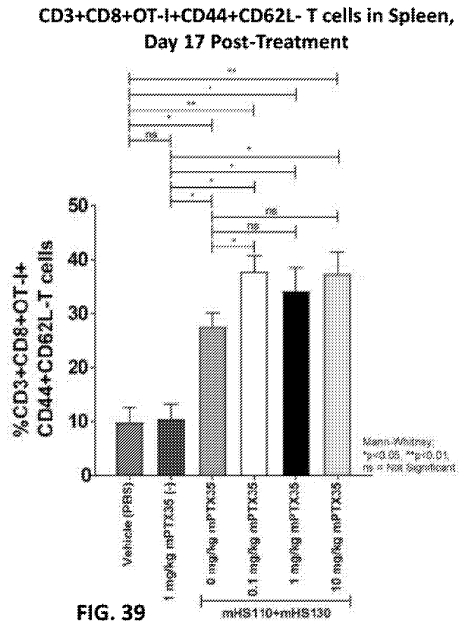

The present study also evaluated the effect of PTX-35 plus mHS-110/mHS-130 on effector memory CD8 T cell responses. C57BL/6 mice (n=5 mice per group) bearing 1B16F110 melanoma tumors and adoptively transferred with one-million syngeneic OT-I transgenic CD8 T cells were sacrificed on day 21 post-tumor inoculation when mice reached tumor burden. FIG. 37 shows representative FACS plots illustrating the percentage of CD3+CD8+ OT-I+ T cells in the spleen on day 17 post-treatment, and FIG. 38 illustrates the percentage of CD3+CD8+ OT-I+ T cells in the spleen on day 17 post-treatment, presented as the mean±SEM of the experiment. FIG. 39 are bar charts illustrating the percentage of CD3+CD8+ OT-I+ effector memory cells (% CD3+CD8+ OT-I⁺ CD44+CD62L− T cells) enumerated during during the peak of CD8 T cell responses on day 17 post-treatment, which are presented as the mean±SEM of an experiment containing 5 mice/group. Mann-Whitney two-tailed test was used to determine statistical significance. *p<0.05, **p<0.01, NS=Not Significant. FIGS. 37, 38 and 39 illustrate that PTX-35 plus mHS-110/mHS-130 augments effector memory CD8 T cell responses.

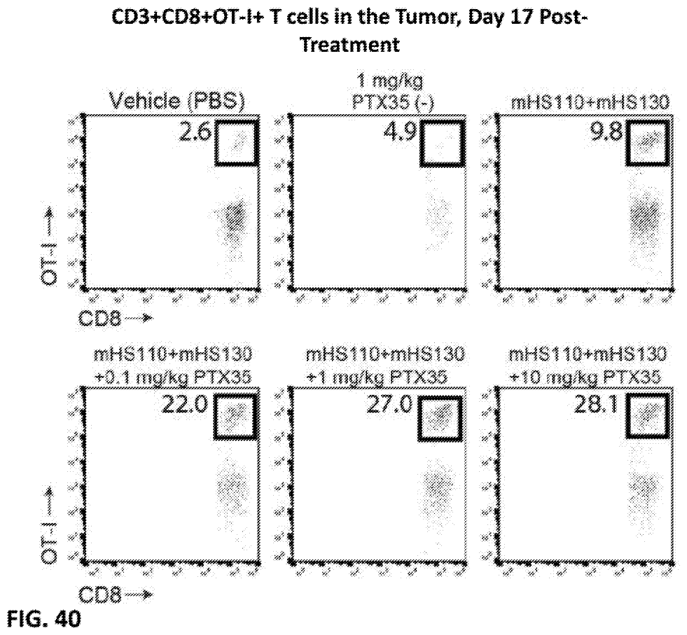

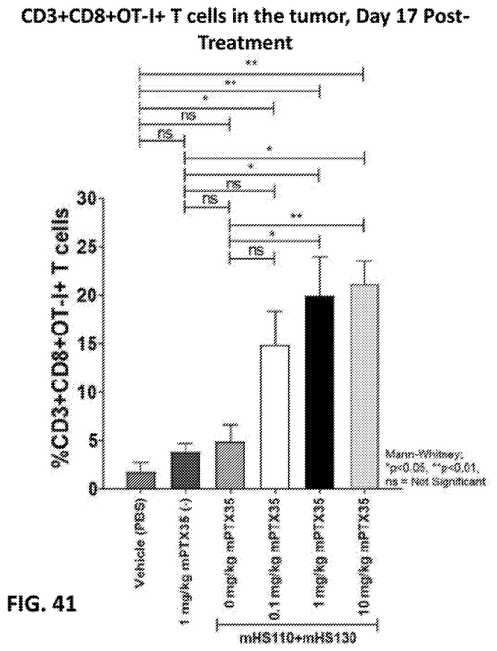

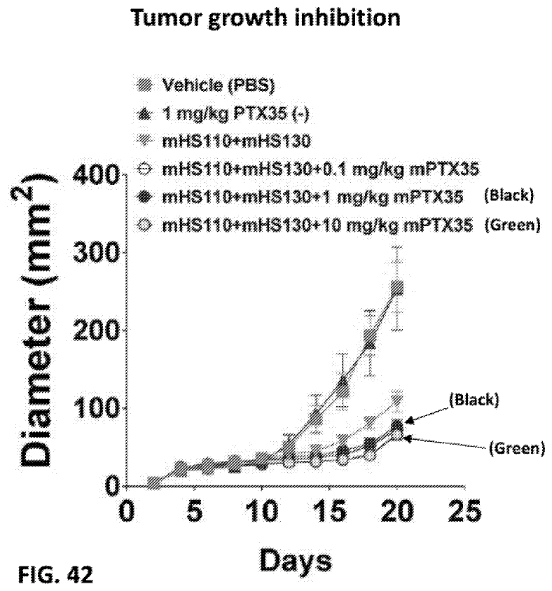

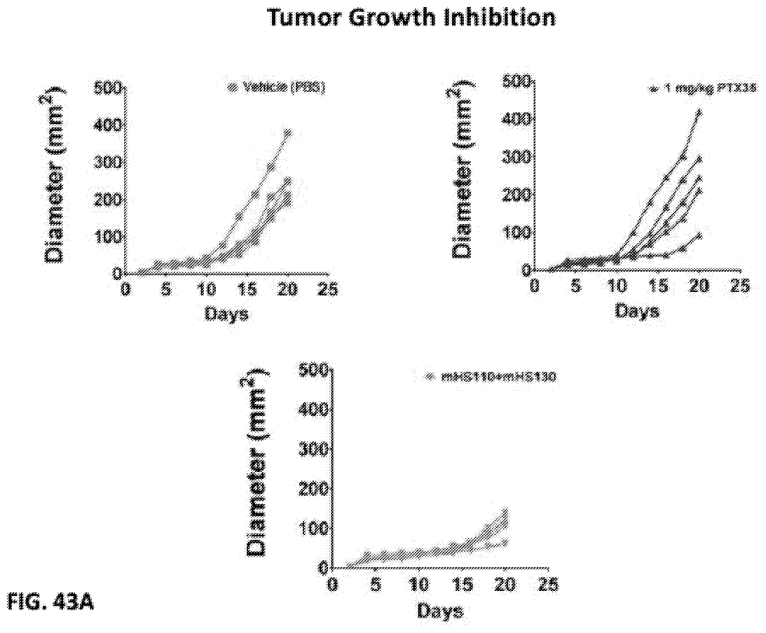

FIG. 40 shows representative FACS plots illustrating the percentage of CD3+CD8+ OT-I⁺ T cells in the tumor on day 17 post-treatment, and FIG. 41 illustrates the percentage of CD3+CD8+ OT-I⁺ T cells in the tumor on day 17 post-treatment, presented as the mean±SEM of the experiment containing 5 mice per group. FIGS. 40 and 41 illustrate that tumor-infiltrating lymphocytes (TILs) are markedly increased after PTX-35 addition.

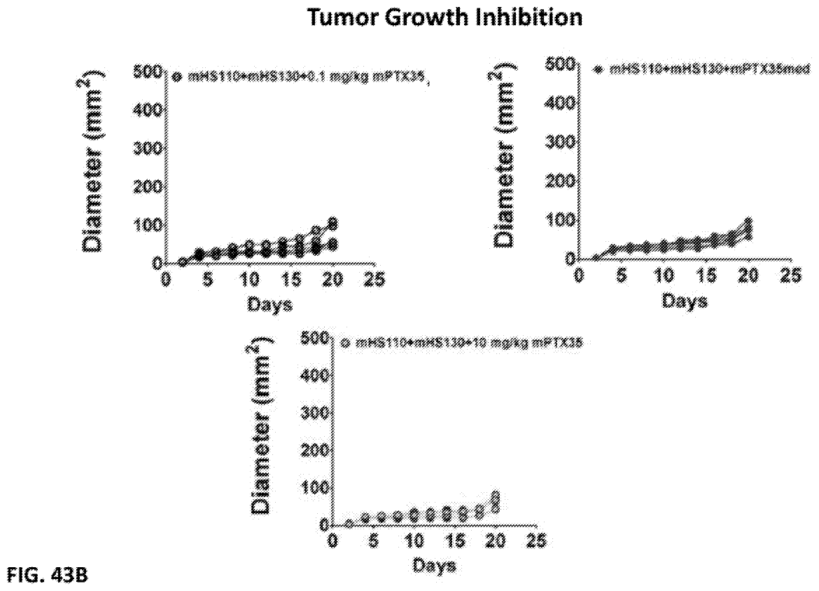

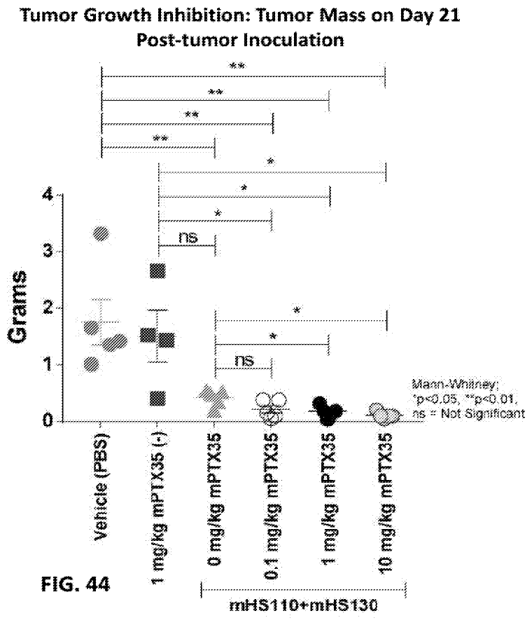

Furthermore, FIGS. 42 to 44 illustrate that the significantly increased levels of TILs result in substantial tumor growth inhibition. The tumor size was measured every 2 days with a caliper, starting on day 2, and calculated using the formula (L×S), where L is the largest diameter of tumor and S is the smallest diameter of tumor. FIG. 42, illustrating tumor growth (in diameter, mm²), over days 2-21 of the study, shows that the tumor diameter was significantly smaller in the mHS-110+mHS-130+10 mg/kg PTX-35 (shown with green circles), mHS-110+mHS-130+1 mg/kg PTX-35 (shown with black circles), and mHS-110+mHS-130+0.1 mg/kg PTX-35 groups (shown with white circles). FIGS. 43A and 43B illustrate tumor growth (in diameter, mm²), over days 2-21. The tumor size was measured every 2 days with a caliper, starting on day 2, for each mouse (n=5) in each of the groups studied. In this study, tumors were extracted on day 21 post-tumor inoculation when mice reached tumor burden and tumor mass (in grams) was recorded, as shown in FIG. 44. Table 4 below illustrates 2-way ANOVA statistics performed to determine statistical significance. *p<0.05, **p<0.01, ***p<0.001, ****p<0.0001, NS=Not Significant.

TABLE 4
Statistics: 2-way ANOVA; group vs. Vehicle (PBS).

| | Day | | | | | | | | | |
|---|---|---|---|---|---|---|---|---|---|---|
| Group | 2 | 4 | 6 | 8 | 10 | 12 | 14 | 16 | 18 | 20 |
| 1 mg/kg PTX-35 (−) | NS | NS | NS | NS | NS | NS | NS | NS | NS | NS |
| mHS-110 + mHS-130 | NS | NS | NS | NS | NS | NS | NS | ** | **** | **** |
| mHS-110 + mHS-130 + 0.1 mg/kg mPTX-35 | NS | NS | NS | NS | NS | NS | NS | *** | **** | **** |
| mHS-110 + mHS-130 + 1 mg/kg mPTX-35 | NS | NS | NS | NS | NS | NS | NS | *** | **** | **** |
| mHS-110 + mHS-130 + 10 mg/kg mPTX-35 | NS | NS | NS | NS | NS | NS | * | **** | **** | **** |

### Example 8: A Study of Synergy of Checkpoint Inhibition (CPI) with Gp96-Ig (mHS-110) and/or PTX-35

The objective of this study was to determine whether the addition of a checkpoint inhibitor to a cancer vaccine and a TNFRSF25 agonist can reduce tumor burden when treatment was provided in a therapeutic manner. More particularly, an impact on tumor growth when treatment included mHS-110, mPTX-35 and a checkpoint inhibitor, anti-PD-1, the dose of which was based on published studies for full blockade of mouse PD-1, was determined.

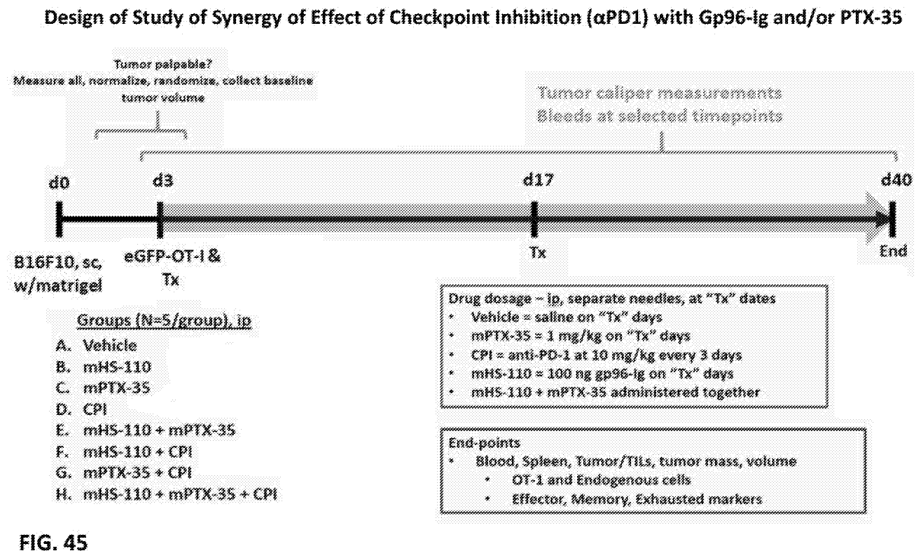

FIG. 45 illustrates the design of the present study. OT-1 purification, adoptive T-cell transfer, mHS-110/mPTX-35 dosing, and flow cytometry staining T-cell receptor (TCR) transgenic mouse CD8+ (OT-I) cells were isolated from in-house bred OT-I-GFP mice using Easy Sep Mouse CD8 T Cell isolation kit (cat #19853A) and injected into each C57BL/6 mouse intravenously (i.v) with 1 million OT-1 cells suspended in HBSS (GIBCO 14175-095). One day after injecting OT-1 cells, all the mice were tail bled for baseline and 4 hours later, mHS-110 (B16F10-OVA-gp96-Ig) cells were treated with 10 μg/mL of Mitomycin-C(Sigma-Aldrich cat #M0503) for 3 hours and given intraperitoneally (i.p.) to each group accordingly along with different doses of mPTX35 (mPTX35low (0.1 mg/kg), mPTX35med (1 mg/kg), and mPTX35hi (10 mg/kg). Mice were divided into 6 groups following groups with 5 mice per group: (A) vehicle, (B) mHS-110, (C) mPTX-35, (D) CPI, (E) mHS-110+mPTX-35, (F) mHS-110+CPI, (G) mPTX-35+CPI, and (H) mHS-110+mPTX-35+CPI, as shown in FIG. 45. The vehicle is saline, mPTX-35 was administered at a dose of 1 mg/kg, CPI=anti-PD1 at 10 mg/kg every 3 days, mHS-110=100 ng gp96-Ig.

Animals were dosed based on nanogram expression level (per 10⁶ cells per 24 hr) of gp96-Ig per Table 5 below.

TABLE 5
Expression of mouse gp96-Ig from mHS-110 per
million cells in a 24-hour period
Mouse gp96-Ig (nanograms/mL per million cells in 24 hrs)

| | Prime Dose: | 1^{st} Boost Dose: | Average |
|---|---|---|---|
| Replicate | Concentration | Concentration | Expression |
| 1 | 79.12 | 68.27 | 95.05 |
| 2 | 77.63 | 66.10 | 100.94 |
| 3 | 76.13 | 66.82 | 97.25 |
| 4 | 76.28 | 69.88 | 100.09 |
| 5 | 77.77 | 68.50 | 97.30 |
| 6 | 76.28 | 67.11 | 93.14 |
| Mean | 77.20 | 67.78 | 97.29 |
| STDEV | 1.18 | 1.36 | 2.94 |
| CV % | 1.53 | 2.01 | 3.02 |

Mice were tail bled consecutively on days 0, 4, 7, 11, and 15 post-immunization into heparinized PBS (10 units/ml) and lysed using ACK lysis buffer (150 mM NH4Cl, 100 mM KHCO3 and 10 mM EDTA 0.2 Na, pH 7.2) for 2 minutes and neutralized with 1×PBS. To distinguish OT-I transferred cells, samples were centrifuged at 300×g for 5 minutes, supernatant was removed and the cell pellet stained with anti-CD3 (20 μg/ml), anti-CD44 (20 μg/ml), anti-CD127 (20 μg/ml), anti-KLRG1 (20 μg/ml) and anti-CD8 (5 μg/ml) antibody cocktail made in FACS buffer using Alexa Fluor 700 anti-mouse CD3 (BioLegend cat #100216), PE-Cy7 anti-mouse CD44 antibody (Biolegend Cat #103030), PE anti-mouse CD127 (BioLegend cat #135010), APC anti-mouse KLRG1 (Biolegend Cat #138412) and Brilliant Violet 421 anti-mouse CD8alpha antibody (BioLegend Cat #100738) for 30 minutes at 4° C. All samples were centrifuged and washed with FACS buffer after staining and 300 μl of FACS buffer was added and 30,000 CD3 gated events were collected on the Sony Flow Cytometer (SH-800).

B16F10-OVA Tumor Challenge and Volume Calculations

Melanoma B16F10 cells were harvested and resuspended at a concentration of 5×10⁵ cells/100 μl in a volume of 80 μl HBSS and 20 μl Matrigel. C57BL/6 mice were subcutaneously injected with 100 μl of B16F10 cells (5×10⁵ cells/mouse) on the inner abdomen 3 days pre-OT-I transfer, as shown in the study design FIG. 45.

Checkpoint Inhibition: Anti-PD-1 Antibody

The present study uses anti-mouse PD-1 antibody (CD279) from BioXCell (Cat #BE0146) at 300 ug per mouse every 3 days by i.p.

Cell Line Protein Expression Data for mHS-110

The amount of murine gp96 protein expressed by the mHS-110 cells was determined by ELISA. For each sample to be tested, one million B16F10-Ova9 parental and mHS-110 cells were plated in a 6-well tissue culture plate in a total volume of 1 ml each. Cells were incubated at 37° C. with 5% CO₂ for 24 hours at which point the supernatants were harvested. The supernatants were then centrifuged at 2500 rpm for 5 minutes to pellet any cell debris. Clarified supernatants were then transferred to new 1.5 ml tubes and stored at −80° C. Each sample tested was from a fresh vial of mHS-110 cells thawed and expanded.

To perform the ELISA, 96-well plates (Corning, cat #9018) were coated with 2 ug/ml of sheep anti-gp96 (R&D Systems, cat #AF7606) in carbonate-bicarbonate buffer. Plates were sealed and stored at 4° C. overnight. Plates were then washed 4 times with 1×TBST (VWR, cat #K873) and then blocked with 1× casein solution (Sigma, cat #B6429) for 1 hour at room temperature. The plates were then washed 4 times with 1×TBST and a human gp96-mouse Fc standard (Thermo, lot #2065447) was prepared in IMDM (Gibco, cat #12440-053) with 10% FBS (Gibco, cat #10082-147). A 1000 ng/ml human-gp96-mFc standard solution was made and 2-fold serial dilutions were performed down to 0.977 ng/ml. Sample supernatants were loaded onto the ELISA plates starting at a 1:2 dilution and then 2-fold serial dilutions were performed to a highest dilution of 1:16. Plates were sealed and incubated for 1 hour at room temperature and then washed 4 times with 1×TBST. The detection antibody, goat anti-mouse IgG (Fc)-HRP (Jackson Immunoresearch, cat #115-036-008) was diluted 1:5,000 (80 ng/ml) in 1×TBST and added to the ELISA plates. Plates were then sealed and incubated in the dark for 1 hour at room temperature. After washing the plates 4 times with 1×TBST, TMB substrate (SeraCare, cat #5120-0076) was added to each well and incubated in dark for 15 minutes at room temperature. Reactions were then stopped with 1N sulfuric acid and plates read on the Biotek ELx800 plate reader. Concentrations of gp96 expressed from each sample were then determined based off the standard curve.

Results

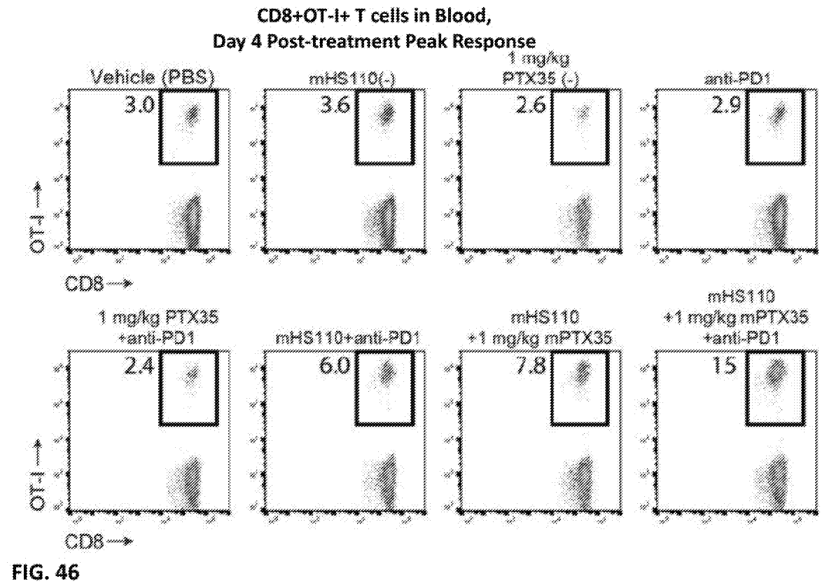

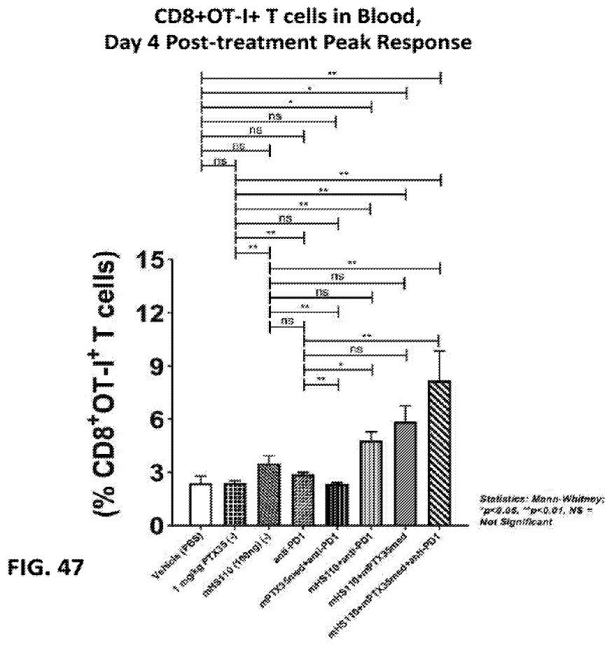

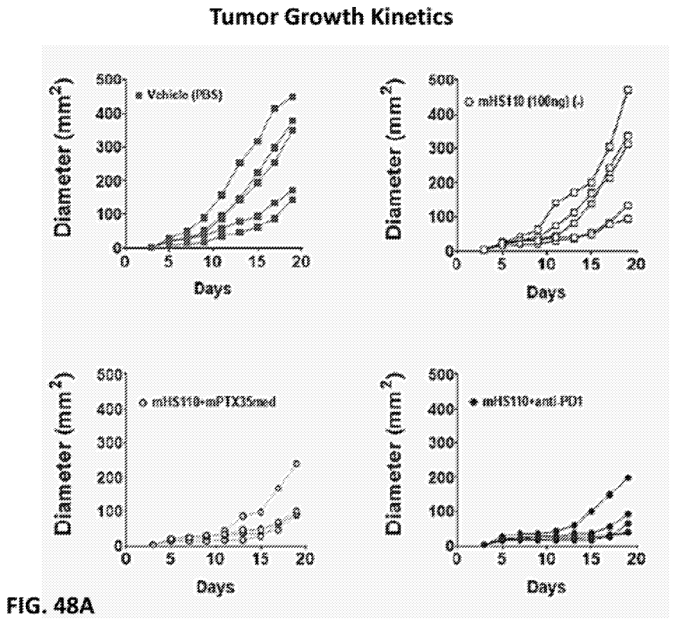

The percentage of CD8+ OT-I+ T cells was enumerated in the peripheral blood during the peak of CD8 T cell responses on day 4 post-treatment. FIGS. 46 and 47 illustrate anti-tumor CD8+ OT-I, T cell expansion, in the peripheral blood with prime and boost immunization of mHS-110 and anti-PD-1 with different doses of mPTX-35. CD8+ OT-I⁺ T cells are presented as a representative FACS plot (FIG. 46) or the mean±SEM of an experiment (FIG. 47). The most prominent cellular expansion of tumor-specific CD8⁺ T-cells was observed when mHS-110 was combined with mPTX-35 and anti-PD-1. Cellular expansion was modest and similar between groups treated with mHS-110 cancer vaccine and mPTX-35 alone vs. anti-PD-1 alone.

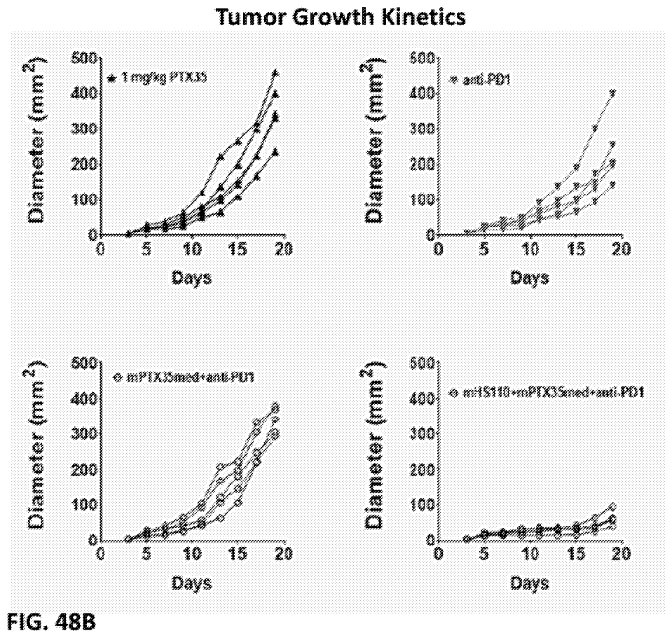

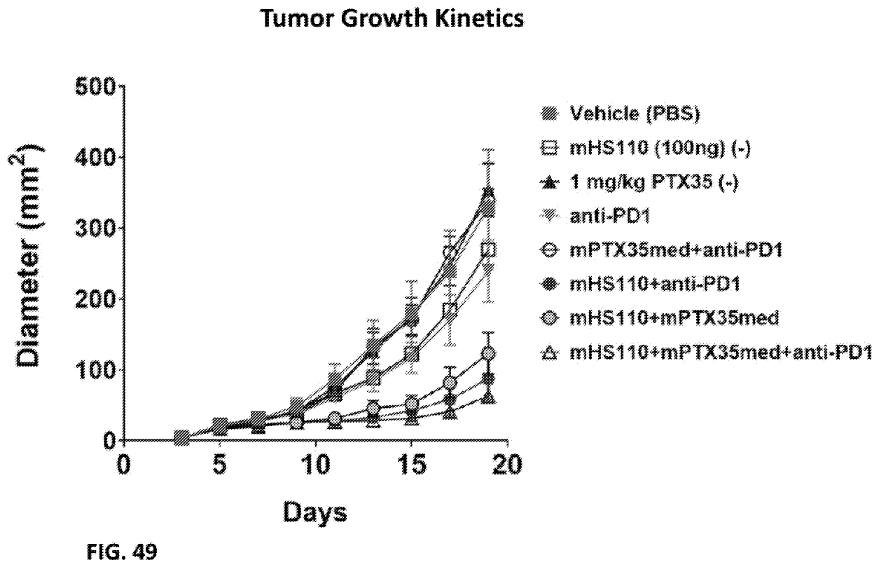

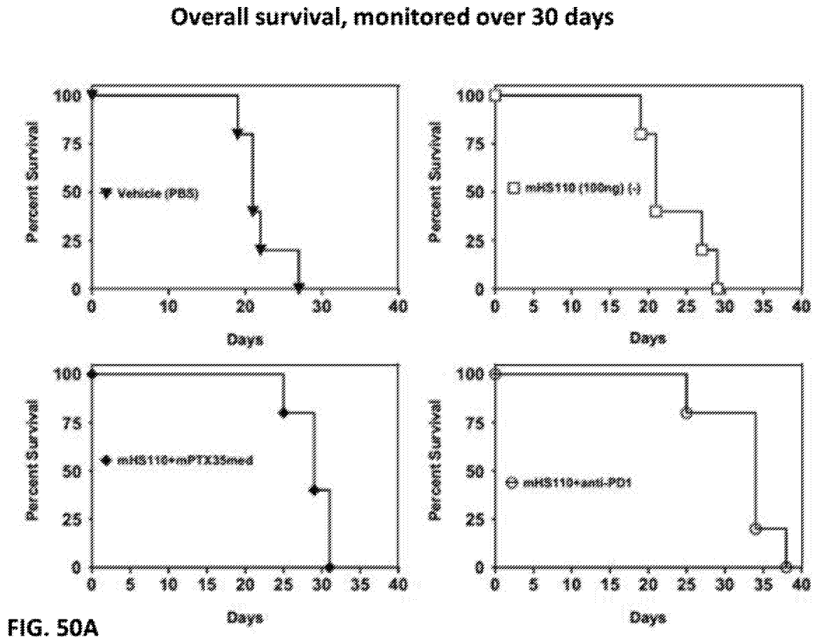

The tumor size was measured with a caliper and documented every 2 days, starting on day 4, and calculated using the formula (A×B) (A as the largest and B as the smallest diameter of tumor). Tumor growth was documented as standard error mean. FIGS. 48A and 48B illustrate tumor growth curves, in diameter (mm²), over days 3-19 for each group of the study of FIG. 45. FIG. 49 illustrates average tumor growth curves, in diameter (mm²), over days 3-219, for each group of the study of FIG. 45. Table 6 below shows a 2-way ANOVA statistics performed to determine statistical significance. *p<0.05, **p<0.01, ***p<0.001, ****p<0.0001, NS=Not Significant.

TABLE 6
Statistics: 2-way ANOVA; group vs. Vehicle (PBS)

| | Day | | | | | | | | |
|---|---|---|---|---|---|---|---|---|---|
| Group | 3 | 5 | 7 | 9 | 11 | 13 | 15 | 17 | 19 |
| mHS-110 (100 ng) (−) | NS | NS | NS | NS | NS | NS | NS | NS | NS |
| 1 mg/kg PTX-35 (−) | NS | NS | NS | NS | NS | NS | NS | NS | NS |
| anti-PD1 | NS | NS | NS | NS | NS | NS | NS | NS | NS |
| 1 mg/kg mPTX-35 + anti-PD1 | NS | NS | NS | NS | NS | NS | NS | NS | NS |
| mHS-110 + anti-PD1 | NS | NS | NS | NS | NS | * | *** | **** | **** |
| mHS-110 + 1 mg/kg mPTX-35 | NS | NS | NS | NS | NS | NS | ** | **** | **** |
| mHS-110 + 1 mg/kg mPTX-35 + anti-PD1 | NS | NS | NS | NS | NS | * | **** | **** | **** |

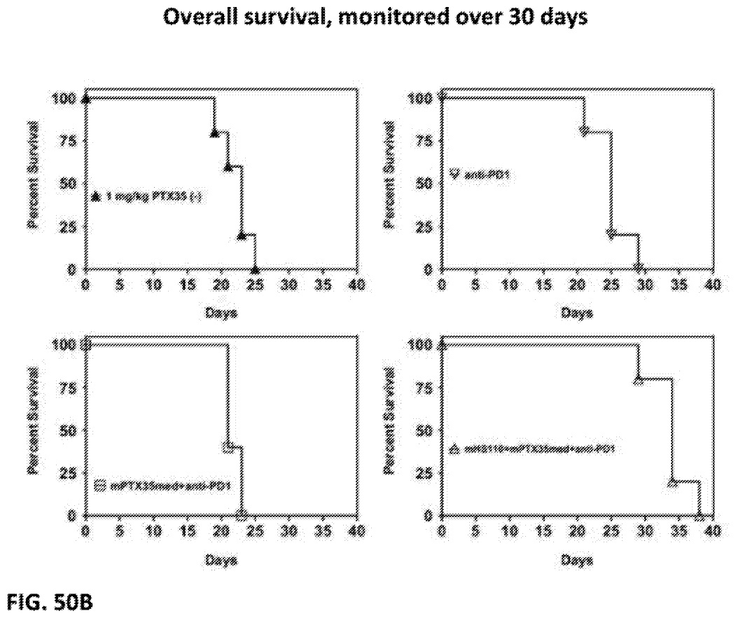

To record the survival of the tumor-bearing mice, either natural death or a tumor volume greater than 450 mm² leading to death was counted as death. Each experimental group included five animals. Survival analysis as graphed by Kaplan-Meier curves showed that tumor growth volume and cellular expansion directly correlated to survival, as shown in FIGS. 50A and 50B illustrating end of study survival curves out to day 40 post-tumor inoculation. The graphs in FIGS. 50A and 50B show mean±SEM, performed statistical analysis was non-parametric t-test, Mann-Whitey (two tailed); *p<0.05, **p<0.01, ***p<0.001, ns=p>0.05=not significant. The most prominent overall survival was observed with the triple (mHS-110+1 mg/kg mPTX-35+anti-PD1) combination (FIG. 50B) and with mHS-110 combined with a checkpoint inhibitor, anti-PD-1 (mHS-110+anti-PD1) (FIG. 50A).

The results of this example demonstrate that checkpoint inhibition (αPD1) with gp96-Ig and/or PTX-35 results in a significant increase in antigen-specific CD8 T cell responses, reduction in tumor growth, and an increase in overall survival. Therapeutic treatment of established B16F10-OVA tumors in mice showed that mHS-110 plus mPTX-35 was effective, and the most prominent activity was observed when mHS-110 was combined with anti-PD-1 and mPTX-35 for tumor volume growth reduction. Thus, it was determined that CPI synergizes effectively with PTX-35 and mHS-110. Therefore, without wishing to be bound by the theory, it can be suggested that combinations of PTX-35 and mHS-110 treatments along with CPI may translate into an efficacious approach to treating human cancers.

In conclusion, 1) mHS-110 combined with checkpoint blockade reproduces what is seen in the clinic for the same combination; 2) mHS-110 combination with a checkpoint inhibitor or TNFRSF25 agonist provides similar efficacy; and 3) the triple combination of mHS-110 plus a checkpoint inhibitor and a TNFRSF25 agonist shows superior activity for both cellular expansion and survival.

### Example 9: CD8⁺ T Cell Expansion Kinetics and Therapeutic Treatment of Established Tumors Using a Combination of mHS-110 (Gp96-Ig), mHS-130 (OX40L-Ig) and mPTX-35 (Anti-TNFRSF25 mAb

The objective of this study was to determine how the triple-treatment combination of mHS-110 with mHS-130 and mPTX-35 compared to the double and single combination of each vaccine and agonist antibody for the expansion of OT-1 CD8+ T-cells, and impact on tumor growth in the therapeutic setting against established tumors. It was determined that the triple combination of two costimulation agonist molecules, OX40L-Ig and anti-TNFRSF25 (mPTX-35) with gp96-Ig significantly impaired the growth of established, aggressive B16.F10-OVA tumors in vivo, and this accompanied expansion of antigen specific CD8+ T-cells.

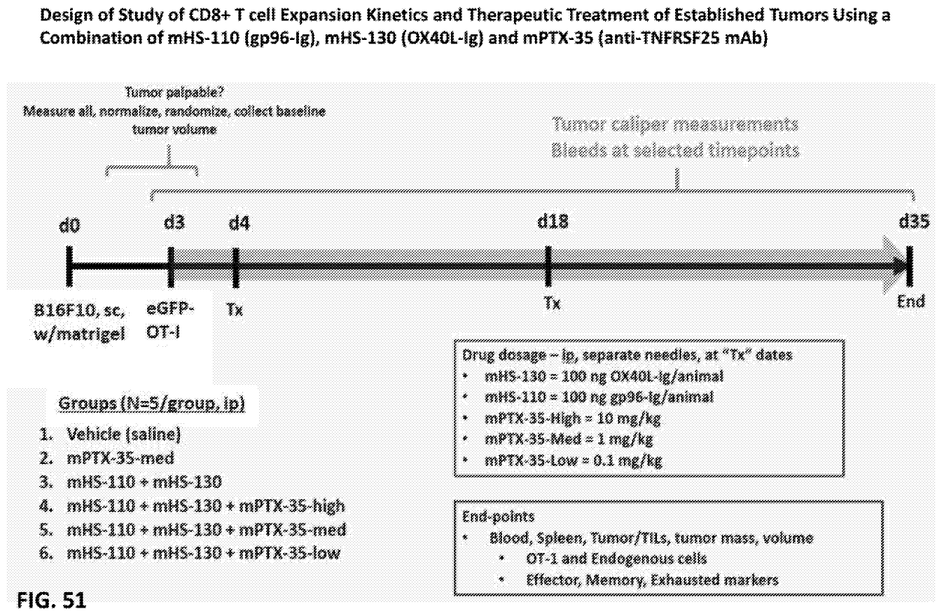

A design of the present study is shown in FIG. 51. T-cell receptor (TCR) transgenic mouse CD8+ (OT-I) cells were isolated from in-house bred OT-I-GFP mice using Easy Sep Mouse CD8 T Cell isolation kit (cat #19853A) and injected into each C57BL/6 mouse intravenously (i.v) with 1 million OT-1 cells suspended in HBSS (GIBCO 14175-095). One day after injecting OT-1 cells, all the mice were tail bled for baseline and 4 hours later, mHS-110 (B16F10-OVA-gp96-Ig) cells were treated with 10 μg/mL of Mitomycin-C(Sigma-Aldrich cat #M0503) for 3 hours and given intraperitoneally (i.p.) to each group accordingly along with different doses of mPTX-35 (mPTX35low (0.1 mg/kg), mPTX35med (1 mg/kg), and mPTX35hi (10 mg/kg). Mice were divided into 6 groups with 5 mice per group. Animals were dosed based on nanogram expression level (per 10⁶ cells per 24 hr) of gp96-Ig per Table 7 below and the study design shown in FIG. 51.

TABLE 7
Expression of mouse gp96-Ig from mHS-130 per
million cells in a 24-hour period
Mouse OX40L-Ig (nanograms/mL per million cells in 24 hrs)

| | Prime Dose: | 1^{st} Boost Dose: | Average |
|---|---|---|---|
| Replicate | Concentration | Concentration | Expression |
| 1 | 339.78 | 391.70 | 365.74 |
| 2 | 371.56 | 424.30 | 397.93 |
| 3 | 378.75 | 388.55 | 383.65 |
| 4 | 416.36 | 447.92 | 432.14 |
| 5 | 402.53 | 452.32 | 427.43 |
| 6 | 408.43 | 403.13 | 405.78 |
| Mean | 386.24 | 417.99 | 402.12 |
| STDEV | 28.62 | 27.90 | 28.26 |
| CV % | 7.41 | 6.68 | 7.05 |

Mice were tail bled consecutively on days 0, 4, 7, 11, and 15 post-immunization into heparinized PBS (10 units/ml) and lysed using ACK lysis buffer (150 mM NH4Cl, 100 mM KHCO3 and 10 mM EDTA 0.2 Na, pH 7.2) for 2 minutes and neutralized with 1×PBS. To distinguish OT-I transferred cells, samples were centrifuged at 300×g for 5 minutes, supernatant was removed and the cell pellet stained with anti-CD3 (20 μg/ml), anti-CD44 (20 μg/ml), anti-CD127 (20 μg/ml), anti-KLRG1 (20 μg/ml) and anti-CD8 (5 μg/ml) antibody cocktail made in FACS buffer using Alexa Fluor 700 anti-mouse CD3 (Biolegend cat #100216), PE-Cy7 anti-mouse CD44 antibody (Biolegend Cat #103030), PE anti-mouse CD127 (Biolegend cat #135010), APC anti-mouse KLRG1 (Biolegend Cat #138412) and Brilliant Violet 421 anti-mouse CD8alpha antibody (Biolegend Cat #100738) for 30 minutes at 4° C. All samples were centrifuged and washed with FACS buffer after staining and 300 μl of FACS buffer was added and 30,000 CD3 gated events were collected on the Sony Flow Cytometer (SH-800).

B16F10-OVA Tumor Challenge and Volume Calculations

Melanoma B16F10 cells were harvested and resuspended at a concentration of 5×10⁵ cells/100 μl in a volume of 80 μl HBSS and 20 μl Matrigel. C57BL/6 mice were subcutaneously injected with 100 μl of B16F10 cells (5×10⁵ cells/mouse) on the inner abdomen 3 days pre-OT-I transfer, as shown in the study design FIG. 51. The tumor size was measured and documented every 2 days with a caliper, starting on day 4, and calculated using the formula (A×B) (A as the largest and B as the smallest diameter of tumor). Tumor growth was documented as standard error mean. To record the survival of the tumor-bearing mice, either natural death or a tumor volume greater than 450 mm² leading to death was counted as death. Each experimental group included five animals.

Tumor Tissue Digestion for Tumor Infiltrating Lymphocytes (TILs)

The MACS Miltenyl Biotec Tumor Dissociation Kit was Used for this Procedure (Cat #130-096-730).

Cell Line Protein Expression Data for mHS-110 and mHS-130

The amount of murine gp96 protein expressed by the mHS-110 cells was determined by ELISA. For each sample to be tested, one million B16F10-Ova9 parental and mHS-110 cells were plated in a 6-well tissue culture plate in a total volume of 1 ml each. Cells were incubated at 37° C. with 5% C02 for 24 hours at which point the supernatants were harvested. Supernatants were then centrifuged at 2500 rpm for 5 minutes to pellet any cell debris. Clarified supernatants were then transferred to new 1.5 ml tubes and stored at −80° C. Each sample tested was from a fresh vial of mHS-110 cells thawed and expanded.

To perform the ELISA, 96-well plates (Corning, cat #9018) were coated with 2 ug/ml of sheep anti-gp96 (R&D Systems, cat #AF7606) in carbonate-bicarbonate buffer. Plates were sealed and stored at 4° C. overnight. Plates were then washed 4 times with 1×TBST (VWR, cat #K873) and then blocked with 1× casein solution (Sigma, cat #B6429) for 1 hour at room temperature. The plates were then washed 4 times with 1×TBST and a human gp96-mouse Fc standard (Thermo, lot #2065447) was prepared in IMDM (Gibco, cat #12440-053) with 10% FBS (Gibco, cat #10082-147). A 1000 ng/ml human-gp96-mFc standard solution was made and 2-fold serial dilutions were performed down to 0.977 ng/ml. Sample supernatants were loaded onto the ELISA plates starting at a 1:2 dilution and then 2-fold serial dilutions were performed to a highest dilution of 1:16. Plates were sealed and incubated for 1 hour at room temperature and then washed 4 times with 1×TBST. The detection antibody, goat anti-mouse IgG (Fc)-HRP (Jackson Immunoresearch, cat #115-036-008) was diluted 1:5,000 (80 ng/ml) in 1×TBST and added to the ELISA plates. Plates were then sealed and incubated in the dark for 1 hour at room temperature. After washing the plates 4 times with 1×TBST, TMB substrate (SeraCare, cat #5120-0076) was added to each well and incubated in dark for 15 minutes at room temperature. Reactions were then stopped with 1N sulfuric acid and plates read on the Biotek ELx800 plate reader. Concentrations of gp96 expressed from each sample were then determined based off the standard curve. This protocol was originally based on the human gp96-Ig protocol #HBI-TM-0005.

The amount of mouse OX40L protein expressed by the mHS-130 cells was also determined by ELISA. For each sample to be tested, one million B16F10-Ova9 parental and mHS-130 cells were plated in a 6-well tissue culture plate in a total volume of 1 ml each. Cells were incubated at 37° C. with 5% C02 for 24 hours at which point the supernatants were harvested. Supernatants were then centrifuged at 2500 rpm for 5 minutes to pellet any cell debris. Clarified supernatants were then transferred to new 1.5 ml tubes and stored at −80° C. Samples collected were from freshly thawed vials of cells.

To perform the ELISA, 96-well plates were coated with 2.5 ug/ml His-tagged mouse OX40 protein (Acro Biosystems, cat #OXO-M5228) in PBS. Plates were sealed and stored at 4° C. overnight. Plates were then washed 4 times with 1×TBST and then blocked with 1% BSA (Sigma, cat #A2153) for 1 hour at room temperature. The plates were then washed 4 times with 1×TBST and a mouse IgG1-mouse OX40L standard (Thermo, lot #2214217) was prepared in IMDM with 10% FBS. A 2000 ng/ml mIgG1-mOX40L standard solution was made and 2-fold serial dilutions were performed down to 1.95 ng/ml. Sample supernatants were loaded onto the ELISA plates and 2-fold serial dilutions were performed. Plates were sealed and incubated for 90 minutes at 37° C. and then washed 4 times with 1×TBST. The detection antibody, goat anti-mouse IgG (Fc)-HRP (Jackson Immunoresearch, cat #115-036-008), was diluted 1:5000 in 1×PBS/0.05% Tween 20/0.1% BSA and added to the ELISA plates. Plates were then sealed and incubated in the dark for 1 hour at room temperature. After washing the plates 4 times with 1×TBST, TMB substrate was added to each well and incubated in dark for 10 minutes at room temperature. Reactions were then stopped with 1N sulfuric acid and plates read on the Biotek ELx800 plate reader. Concentrations of OX40L expressed from each sample were then determined based off the standard curve.

Results

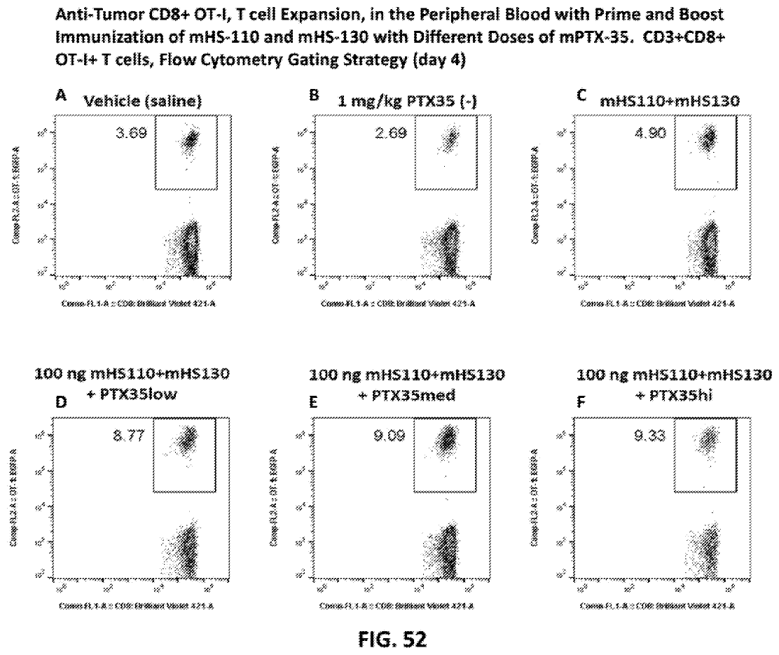

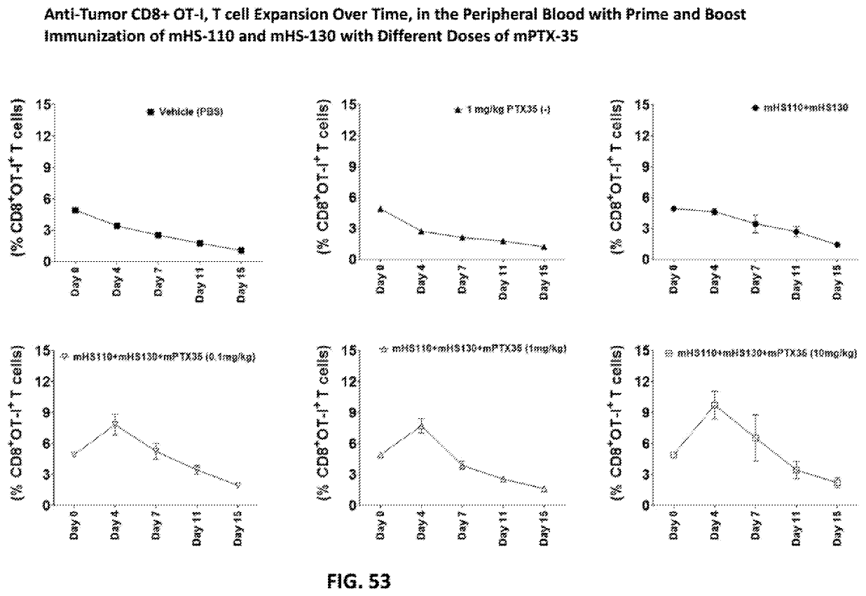

As shown in FIGS. 52 and 53, combination with mPTX-35 and anti-cancer vaccination led to tumor growth inhibition directly proportional to anti-tumor CD8⁺ T-cell expansion. Representative flow cytometry dot plot examples (the peak expansion, day 4) are shown in FIG. 52 illustrating anti-tumor CD8+ OT-I T cell expansion, in the peripheral blood with immunization with mHS-110 and mHS-130 with different doses of mPTX-35, in the following groups: Vehicle (PBS) (panel A), PTX-35 (1 mg/kg) (panel B), mHS-110+mHS-130 (panel C), mHS-110+mHS-130+mPTX-35 (0.1 mg/kg, “low”) (panel D), mHS-110+mHS-130+mPTX-35 (1 mg/kg, “med”) (panel E), and mHS-110+mHS-130+mPTX-35 (10 mg/kg, “hi”) (panel F). Total live cells were gated by SSC and FSC parameters, then cells were gated on CD3+ T-cell events, then gated by eGFP+ OT-1+ CD8+ T cell events.

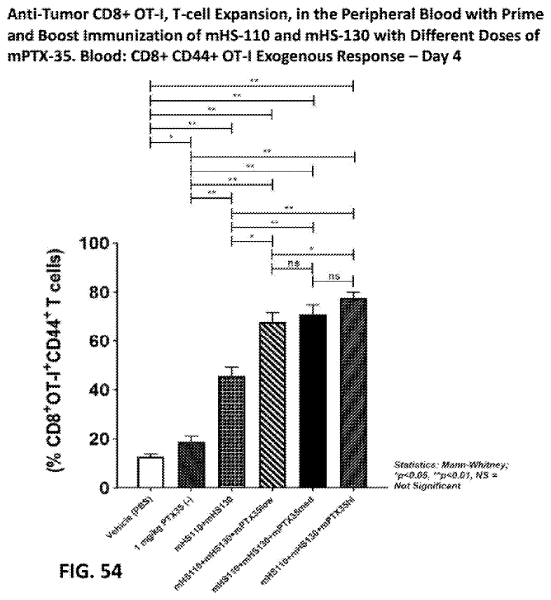

In this study, the peak expansion with mHS-110 and mHS-130 co-immunization occurred on day 4, as shown in FIG. 53 showing summary line graphs illustrating anti-tumor CD8+ OT-I T cell expansion over time (graphs show mean±SEM for each group on each day by peripheral blood compartment). The most prominent T cell expansion was observed for the 1 mg/kg dose of mPTX-35 in combination with OX40L-Ig and gp96-Ig (FIG. 53). However, the expansion of such CD8+ T-cells was similar between all three doses of mPTX-35, including 0.1 mg/kg, which may suggest that the optimal immunology dose may be lower. Effector T-cell generation was directly proportional to TNFRSF25 agonist exposure, when combined with OX40L-Ig and gp96-Ig secreting cellular vaccines, as shown in FIG. 54 illustrating OT-1 cells gated by CD44^{hi} events on day 4, in the peripheral blood.

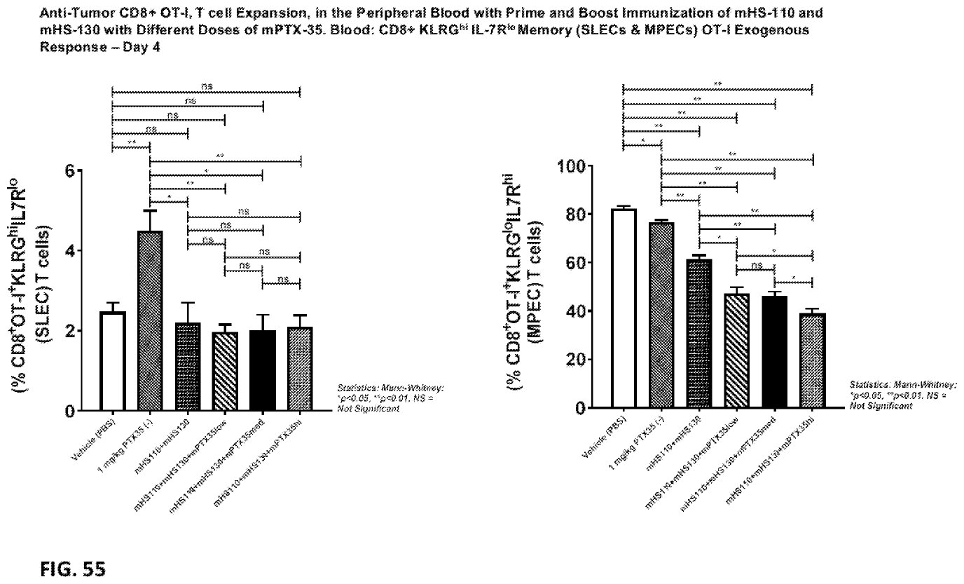

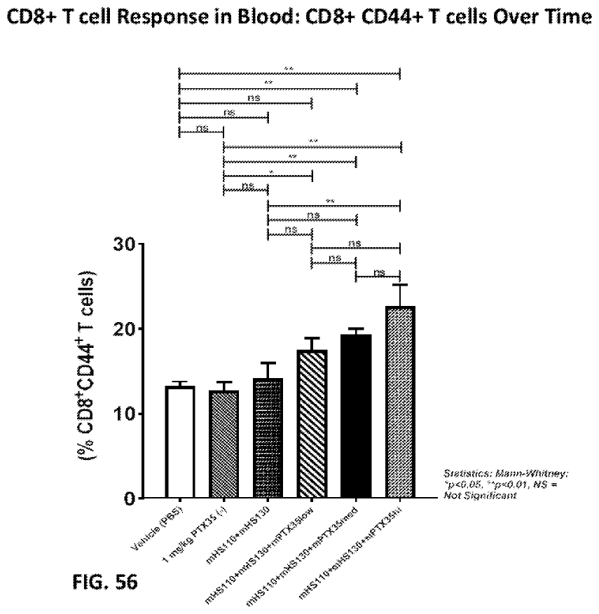

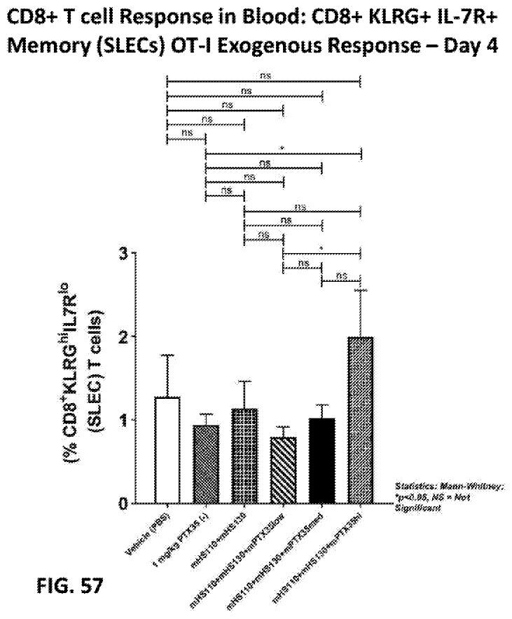

FIG. 55 illustrates CD8+KLRG^{hi} IL-7R^{lo} memory cells, exogenous response on day 4, where short-lived effector cells (SLECs) are gated by the Y-axis markers (left graph), and memory precursor effector cells (MPECs) are gated by the Y-axis markers (right graph). In the peripheral blood, TNFRSF25 agonism, when combined with vaccination, did not change the generation of SLECs, however, treatment with mPTX-35 alone, did significantly increase the generation of SLECs (FIG. 55, left graph). In the peripheral blood, MPECs, decreased in frequency with the addition of mPTX-35 in combination with vaccination (FIG. 55, right graph). In the peripheral blood, endogenous T cell response (% CD8+CD44+ T cells) demonstrates that overall effector CD8+ T cells expanded when mPTX-35 was combined with cancer vaccines, mHS-110 and mHS-130 (FIG. 56). However, there was a modest change in SLECs for endogenous CD8+ T-cells between groups, as shown in FIG. 57.

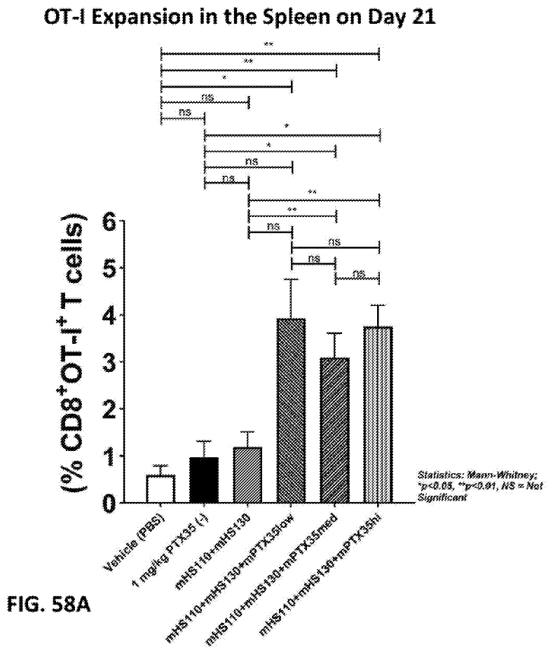

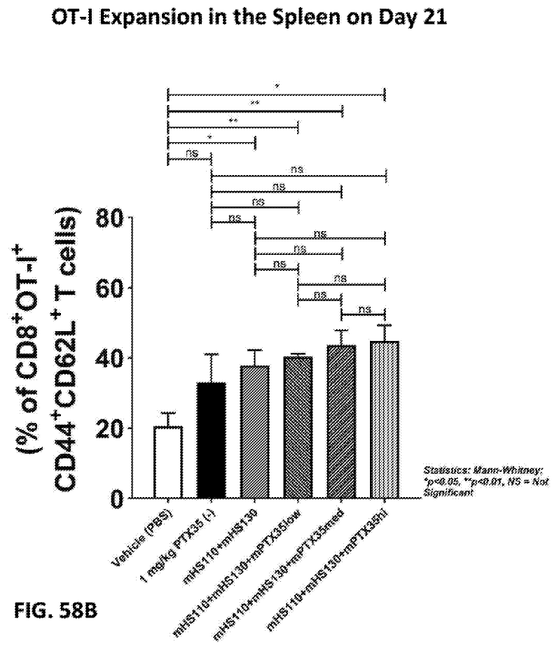

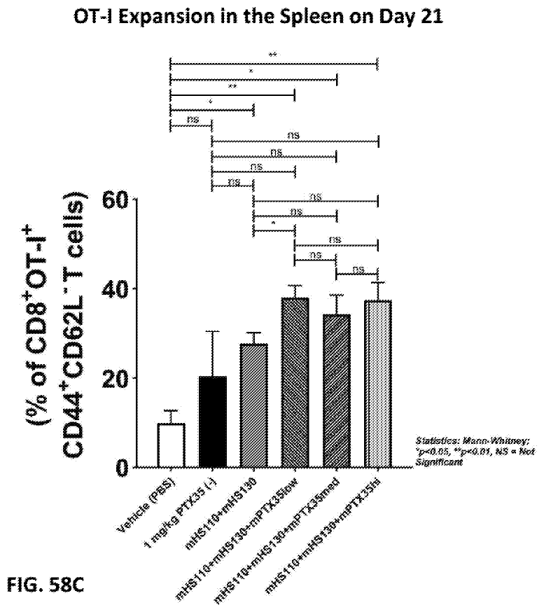

In the spleen at study end, day 21, anti-tumor CD8+ OT-1+ T cells expanded in a dose dependent manner when mPTX-35 was combined with gp96-Ig and OX40L-Ig cancer vaccines (FIG. 58A). This trend continued with the generation of anti-tumor specific effector and central memory T-cells (FIGS. 58B and 58C). The generation of MPECs was indirectly proportional to the generation of SLECs, which makes sense as the two populations share similar markers for identification (KLRG1 and IL-7R). In FIGS. 58A to 58C, bars show mean±SEM, statistical analysis performed was non-parametric t-test, Mann-Whitey, two tailed. *p<0.05, **p<0.01, ***p<0.001, ns=p>0.05=not significant.

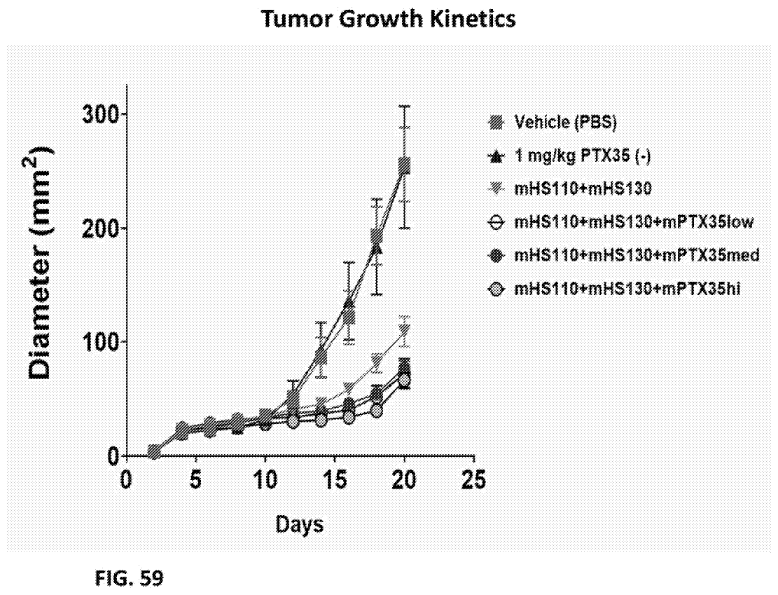

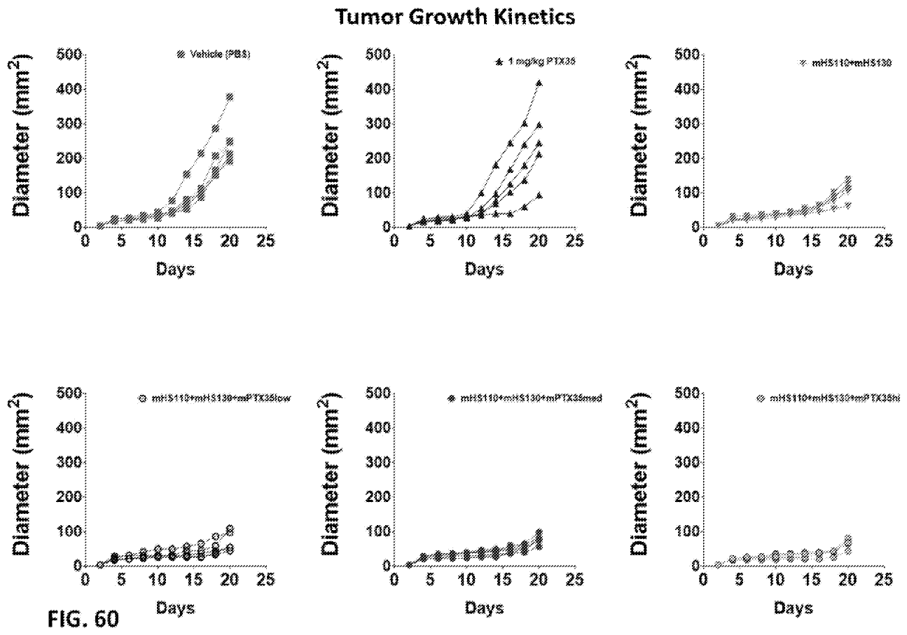

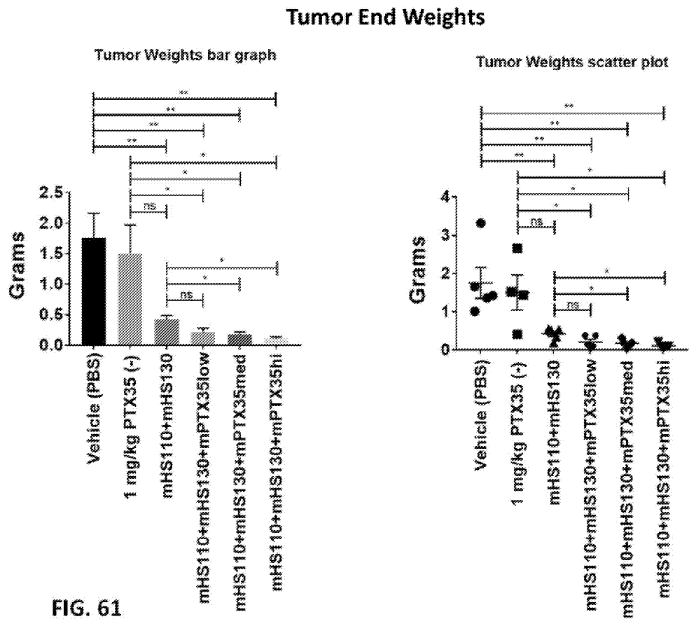

Tumor growth regression was most striking when mPTX-35 was combined with mHS-110 and mHS-130 at 0.1 to 10 mg/kg, suggesting that the optimal immunologic dose may be lower than 0.1 mg/kg (FIGS. 59 and 60). Therapeutic tumor growth inhibition was directly proportional to endpoint tumor weights, as shown in FIG. 61.

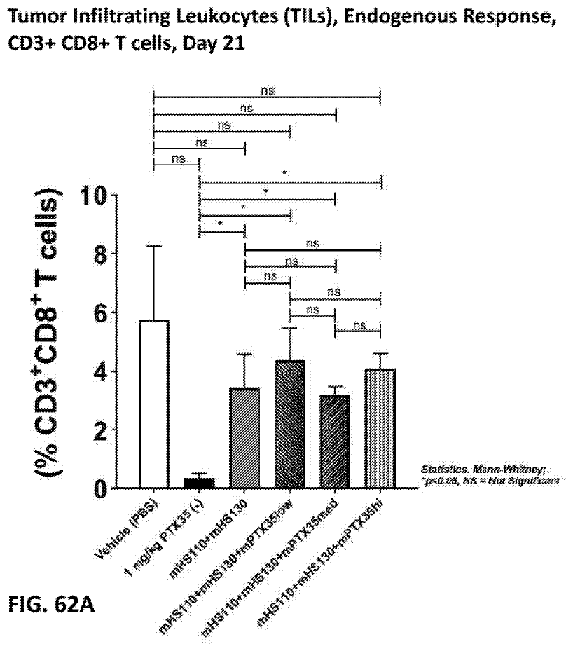

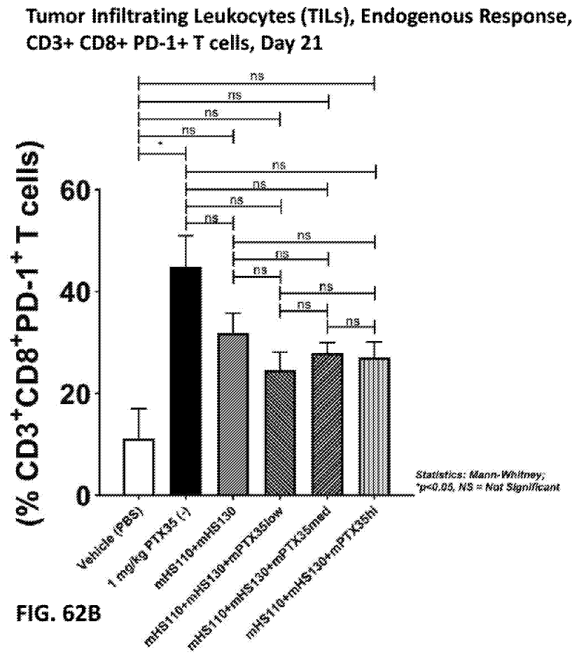

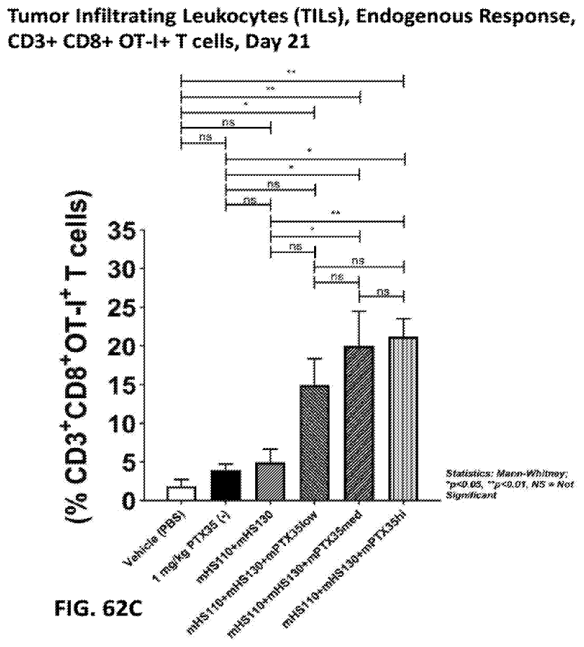

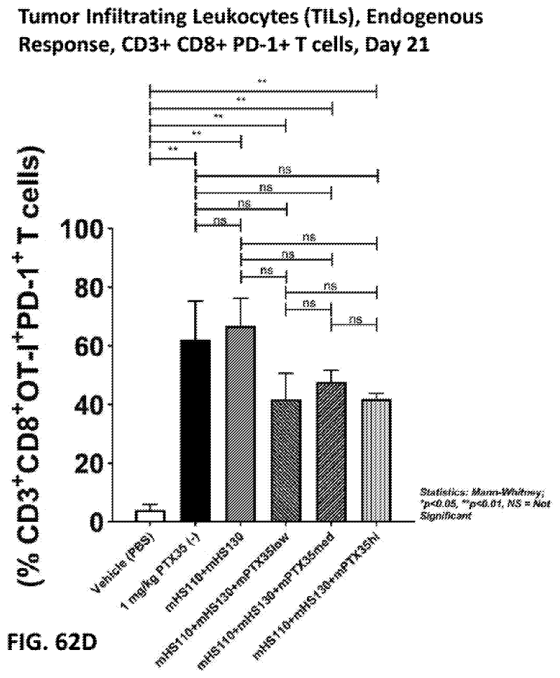

FIGS. 62A to 62D illustrate tumor infiltrating leukocytes (TILs) for endogenous T cells on day 21 of the present study, where FIG. 62A shows percent of CD3+CD8+ T cells, FIG. 62B shows percent of CD3+CD8+PD-1+ T cells, FIG. 62C shows percent of CD3+CD8+ OT-I+ T cells, and FIG. 62D shows percent of CD3+CD8+ OT-I+PD-1+ T cells. The graphs in FIGS. 62A to 62D show mean±SEM of the respective values, and the performed statistical analysis was non-parametric t-test, Mann-Whitey, two tailed; *p<0.05, **p<0.01, ***p<0.001, ns=p>0.05=not significant.

The TILs response showed that endogenous, non-specific T cells were attempting to control tumor burden, but, only the specific cells, OT-1 cells, were able to do so at proportions that were indirectly related to tumor growth (see FIGS. 62A and 62C). There exists an interesting correlation between the TIL percentage, treatment, type of T-cell and tumor burden. A lower percent of CD8⁺ T cells was required to control tumor burden, only when these cells were specific, as shown in FIGS. 62A-62D. Another trend that is observed with mPTX-35 treatment alone is that PD-1 expression increases on TILs (FIGS. 62B and 62D). Without wishing to be bound by a theory, this can be explained by exhaustion or another factor related to the tumor microenvironment (TME) or Treg impact.

In conclusion, results of this study demonstrate, inter alia, that 1) a triple combination of TNFRSF25 agonism with gp96-Ig and OX40L-Ig cancer vaccination is a powerful method to control tumor burden in a therapeutic setting; and 2) the triple combination shows improved synergistic activity over that of a double treatment alone with gp96/OX40L-Ig or gp96-Ig/TNFRSF25.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

### INCORPORATION BY REFERENCE

All patents and publications referenced herein are hereby incorporated by reference in their entireties. The publications discussed herein are provided solely for their disclosure prior to the filing date of the present application. Nothing herein is to be construed as an admission that the present invention is not entitled to antedate such publication by virtue of prior invention. As used herein, all headings are simply for organization and are not intended to limit the disclosure in anyway.

What is claimed is:

1. A method for treating cancer, comprising:
(a) administering an effective amount of an TNF Receptor Superfamily Member 25 (TNFRSF25) agonistic antibody or antigen binding fragment thereof to a patient in need thereof;
(b) assaying a sample from the patient for a T cell modulatory effect; and
(c) administering a second therapy to the patient based on the results of step (b).

2. The method of claim 1, wherein the T cell modulatory effect is an expansion of CD4⁺ T cells and CD8⁺ T cells.

3. The method of claim 2, wherein the expansion provides a ratio of CD4⁺ T cells to CD8⁺ T cells of about 1:1.

4. The method of any one of claims 1-3, wherein the T cell modulatory effect is a differential expansion of effector memory and central memory T cells.

5. The method of claim 4, wherein the expansion provides an increase in a ratio of CD4⁺ central memory T cells to CD4⁺ effector memory T cells.

6. The method of claim 4 or claim 5, wherein the expansion provides an increase in a ratio of CD8⁺ effector memory to CD8⁺ central memory T cells.

7. The method of any one of claims 1-6, wherein the second therapy is a checkpoint inhibitor.

8. The method of claim 7, wherein the checkpoint inhibitor is an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2.

9. The method of claim 8, wherein the agent that targets PD-1 is an antibody or antibody format specific for PD-1, optionally selected from nivolumab, pembrolizumab, and pidilizumab.

10. The method of claim 8, wherein the agent that targets PD-L1 is an antibody or antibody format specific for PD-L1, optionally selected from atezolizumab, avelumab, durvalumab, and BMS-936559.

11. The method of claim 8, wherein the agent that targets CTLA-4 is an antibody or antibody format specific for CTLA-4, optionally selected from ipilimumab and tremelimumab.

12. The method of any one of claims 1-6, wherein the second therapy is radiation therapy.

13. The method of any one of claims 1-6, wherein the second therapy is further administration of the TNFRSF25 agonistic antibody or antigen binding fragment thereof.

14. The method of any one of claims 1-13, wherein the TNFRSF25 agonistic antibody or antigen binding fragment thereof comprises:
(i) a heavy chain variable region comprising heavy chain CDR1, CDR2, and CDR3 sequences, wherein the heavy chain CDR1 sequence is GFTFSNHDLN (SEQ ID NO: 1) or a variant thereof, the heavy chain CDR2 sequence is YISSASGLISYADAVRG (SEQ ID NO: 2) or a variant thereof; and the heavy chain CDR3 sequence is DPAYTGLYALDF (SEQ ID NO: 3) or a variant thereof, or DPPYSGLYALDF (SEQ ID NO: 4) or a variant thereof; and
(ii) a light chain variable region comprising light chain CDR1, CDR2, and CDR3 sequences, wherein the light chain CDR1 sequence is TLSSELSWYTIV (SEQ ID NO: 5) or a variant thereof, the light chain CDR2 sequence is LKSDGSHSKGD (SEQ ID NO: 6) or a variant thereof, and the light chain CDR3 sequence is CGAGYTLAGQYGWV (SEQ ID NO: 7) or a variant thereof.

15. The method of claim 14, wherein the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

16. The method of claim 15, wherein the variable region FW sequences are human.

17. The method of any one of claims 1-16, wherein the TNFRSF25 agonistic antibody or antigen binding fragment thereof further comprises human heavy chain and light chain constant regions.

18. The method of claim 17, wherein the constant regions are selected from the group consisting of human IgG1, IgG2, IgG3, and IgG4.

19. The method of claim 18, wherein the constant regions are IgG1.

20. The method of claim 18, wherein the constant regions are IgG4.

21. The method of any one of claims 1-20, wherein the TNFRSF25 agonistic antibody or antigen binding fragment thereof comprises a heavy chain variable region of the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of about 85% to about 99% identity thereto.

22. The method of any one of claims 1-21, wherein the TNFRSF25 agonistic antibody or antigen binding fragment thereof comprises a light chain variable region of the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of about 85% to about 99% identity thereto.

23. The method of any one of claims 1-6, wherein the second therapy is a biological adjuvant.

24. The method of claim 23, wherein the biological adjuvant comprises a secretable vaccine protein.

25. The method of claim 24, wherein the secretable vaccine protein is gp96.

26. The method of claim 24 or claim 25, wherein the secretable vaccine protein is a gp96-Ig fusion protein.

27. The method of claim 26, wherein the gp96-Ig fusion protein lacks the gp96 KDEL (SEQ ID NO: 10) sequence.

28. The method of claim 26 or claim 27, wherein the Ig tag in the gp96-Ig fusion protein comprises the Fc region of human IgG1, IgG2, IgG3, IgG4, IgM, IgA, or IgE.

29. The method of any one of claims 23-28, wherein the biological adjuvant further comprises a T cell costimulatory fusion protein which enhances activation of antigen-specific T cells.

30. The method of claim 29, wherein the T cell costimulatory fusion protein is selected from OX40L-Ig, or a portion thereof that binds to OX40, ICOSL-Ig, or a portion thereof that binds to ICOS, 4 1BBL-Ig, or a portion thereof that binds to 4-1BBR, TL1A-Ig, or a portion thereof that binds to TNFRSF25, GITRL-Ig, or a portion thereof that binds to GITR, CD40L-Ig, or a portion thereof that binds to CD40, and, CD70-Ig, or a portion thereof that binds to CD27.

31. The method of claim 29, wherein the T cell costimulatory fusion protein is an Ig fusion protein.

32. The method of any one of claims 29-31, wherein the Ig tag in the T cell costimulatory fusion protein comprises the Fc region of human IgG1, IgG2, IgG3, IgG4, IgM, IgA, or IgE.

33. The method of claim 29, wherein the T cell costimulatory fusion protein is OX40L-Ig administered in combination with the gp96-Ig fusion protein lacking the gp96 KDEL (SEQ ID NO: 10) sequence.

34. The method of any one of claims 1-33, further comprising administering a checkpoint inhibitor.

35. The method of claim 34, wherein the checkpoint inhibitor is an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2.

36. The method of claim 35, wherein the checkpoint inhibitor is an agent that targets PD-1.

37. The method of claim 33, wherein the OX40L-Ig fusion protein and the gp96-Ig fusion protein are secreted by a single cell line.

38. The method of claim 33, wherein the OX40L-Ig fusion protein and the gp96-Ig fusion protein are secreted by respective separate cell lines.

39. The method of any one of claims 29-38, wherein the secretable vaccine protein and/or T cell costimulatory fusion protein are encoded on an expression vector.

40. The method of claim 39, wherein the expression vector is incorporated into a human tumor cell.

41. The method of claim 40, wherein the human tumor cell is an irradiated or live and attenuated human tumor cell.

42. The method of claim 40 or claim 41, wherein the human tumor cell is a cell from an established NSCLC, bladder cancer, melanoma, ovarian cancer, renal cell carcinoma, prostate carcinoma, sarcoma, breast carcinoma, squamous cell carcinoma, head and neck carcinoma, hepatocellular carcinoma, pancreatic carcinoma, or colon carcinoma cell line.

43. The method of any one of claims 1-6, wherein the second therapy is chemotherapy.

44. The method of claim 43, wherein the chemotherapy is selected from alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above.

45. The method of any one of claims 1-44, wherein the sample from the patient is selected from a tissue biopsy, tumor biopsy, tumor resection, frozen tumor tissue specimen, lymph node, bone marrow, circulating tumor cells, cultured cells, a formalin-fixed paraffin embedded tumor tissue specimen, and combinations thereof.

46. The method of claim 45, wherein the biopsy is selected from a core biopsy, needle biopsy, surgical biopsy, and an excisional biopsy.

47. The method of any one of claims 1-46, wherein the assay is a measurement of cytokine levels, cytokine secretion, surface markers, cytolytic protein secretion, and/or genomic profiles.

48. The method of any one of claims 1-47, wherein the assay employs cytoplasmic dyes, optionally being carboxyfluorescein succinimidyl ester (CFSE).

49. The method of claim 48, wherein the assay employs CFSE and measures cell proliferation.

50. The method of any one of claims 1-49 wherein the assay employs one or more of ELISPOT (enzyme-linked immunospot), intracellular cytokine staining (ICS), fluorescence-activated cell sorting, (FACS), microfluidics, PCR, and nucleic acid sequencing.

51. The method of any one of claims 1-50, wherein the assay employs analysis of surface markers such as CD45RA/RO isoforms and CCR7 or CD62L expression.

52. The method of any one of claims 1-51, wherein the assay is a measurement of cytokine levels and/or cytokine secretion, and the cytokine is selected from one or more of IFN-γ, TNF, and IL-2.

53. The method of any one of claims 1-52, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

54. A method for treating cancer, comprising:
(a) administering an effective amount of an TNF Receptor Superfamily Member 25 (TNFRSF25) agonistic antibody or antigen binding fragment thereof to a patient in need thereof;
(b) administering a biological adjuvant; and
(c) administering a checkpoint inhibitor molecule.

55. The method of claim 54, wherein the checkpoint inhibitor is an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2.

56. The method of claim 55, wherein the agent that targets PD-1 is an antibody or antibody format specific for PD-1.

57. The method of claim 56, wherein the agent that targets PD-1 is optionally selected from nivolumab, pembrolizumab, and pidilizumab.

58. The method of claim 55, wherein the agent that targets PD-L1 is optionally selected from atezolizumab, avelumab, durvalumab, and BMS-936559.

59. The method of claim 55, wherein the agent that targets CTLA-4 is an antibody or antibody format specific for CTLA-4, optionally selected from ipilimumab and tremelimumab.

60. The method of any one of claims 54-59, wherein the TNFRSF25 agonistic antibody or antigen binding fragment thereof comprises:
(i) a heavy chain variable region comprising heavy chain CDR1, CDR2, and CDR3 sequences, wherein the heavy chain CDR1 sequence is GFTFSNHDLN (SEQ ID NO: 1) or a variant thereof, the heavy chain CDR2 sequence is YISSASGLISYADAVRG (SEQ ID NO: 2) or a variant thereof; and the heavy chain CDR3 sequence is DPAYTGLYALDF (SEQ ID NO: 3) or a variant thereof, or DPPYSGLYALDF (SEQ ID NO: 4) or a variant thereof; and
(ii) a light chain variable region comprising light chain CDR1, CDR2, and CDR3 sequences, wherein the light chain CDR1 sequence is TLSSELSWYTIV (SEQ ID NO: 5) or a variant thereof, the light chain CDR2 sequence is LKSDGSHSKGD (SEQ ID NO: 6) or a variant thereof, and the light chain CDR3 sequence is CGAGYTLAGQYGWV (SEQ ID NO: 7) or a variant thereof.

61. The method of claim 60, wherein the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

62. The method of claim 61, wherein the variable region FW sequences are human.

63. The method of any one of claims 54-62, wherein the TNFRSF25 agonistic antibody or antigen binding fragment thereof further comprises human heavy chain and light chain constant regions.

64. The method of claim 63, wherein the constant regions are selected from the group consisting of human IgG1, IgG2, IgG3, and IgG4.

65. The method of claim 64, wherein the constant regions are IgG1.

66. The method of claim 64, wherein the constant regions are IgG4.

67. The method of any one of claims 54-66, wherein the TNFRSF25 agonistic antibody or antigen binding fragment thereof comprises a heavy chain variable region of the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of about 85% to about 99% identity thereto.

68. The method of any one of claims 54-67, wherein the TNFRSF25 agonistic antibody or antigen binding fragment thereof comprises a light chain variable region of the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of about 85% to about 99% identity thereto.

69. The method of any one of claims 54-68, wherein the biological adjuvant comprises a secretable vaccine protein.

70. The method of claim 69, wherein the secretable vaccine protein is gp96.

71. The method of claim 69 or 70, wherein the secretable vaccine protein is a gp96-Ig fusion protein.

72. The method of claim 71, wherein the gp96-Ig fusion protein lacks the gp96 KDEL (SEQ ID NO: 10) sequence.

73. The method of claim 71 or claim 72, wherein the Ig tag in the gp96-Ig fusion protein comprises the Fc region of human IgG1, IgG2, IgG3, IgG4, IgM, IgA, or IgE.

74. The method of any one of claims 69-73, wherein the biological adjuvant further comprises a T cell costimulatory fusion protein which enhances activation of antigen-specific T cells.

75. The method of claim 74, wherein the T cell costimulatory fusion protein is selected from OX40L-Ig, or a portion thereof that binds to OX40, ICOSL-Ig, or a portion thereof that binds to ICOS, 4 1BBL-Ig, or a portion thereof that binds to 4-1BBR, TL1A-Ig, or a portion thereof that binds to TNFRSF25, GITRL-Ig, or a portion thereof that binds to GITR, CD40L-Ig, or a portion thereof that binds to CD40, and, CD70-Ig, or a portion thereof that binds to CD27.

76. The method of claim 75, wherein the T cell costimulatory fusion protein is an Ig fusion protein.

77. The method of any one of claims 74-76, wherein the Ig tag in the T cell costimulatory fusion protein comprises the Fc region of human IgG1, IgG2, IgG3, IgG4, IgM, IgA, or IgE.

78. The method of claim 75 or claim 76, wherein the T cell costimulatory fusion protein is OX40L-Ig administered in combination with the gp96-Ig fusion protein lacking the gp96 KDEL (SEQ ID NO: 10) sequence.

79. The method of any one of claims 54-78, wherein the method results in an increase in an antigen-specific CD8 T cell response in the patient.

80. The method of any one of claims 54-79, wherein the method results in an increase in a number of tumor-infiltrating lymphocytes in the patient.

81. The method of any one of claims 54-80, wherein the method results in a decrease in at least one of a tumor size and a tumor growth rate in the patient.

82. A method for treating cancer, comprising:
(a) administering an effective amount of an TNF Receptor Superfamily Member 25 (TNFRSF25) agonistic antibody or antigen binding fragment thereof to a patient in need thereof;
(b) administering a biological adjuvant to the patient;
wherein the patient is undergoing treatment with a checkpoint inhibitor molecule.

83. The method of claim 82, wherein the checkpoint inhibitor is an agent that targets one of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2.

84. The method of claim 83, wherein the agent that targets PD-1 is an antibody or antibody format specific for PD-1.

85. The method of claim 84, wherein the agent that targets PD-1 is optionally selected from nivolumab, pembrolizumab, and pidilizumab.

86. The method of claim 83, wherein the agent that targets PD-L1 is optionally selected from atezolizumab, avelumab, durvalumab, and BMS-936559.

87. The method of any one of claims 82-86, wherein the TNFRSF25 agonistic antibody or antigen binding fragment thereof comprises:
(i) a heavy chain variable region comprising heavy chain CDR1, CDR2, and CDR3 sequences, wherein the heavy chain CDR1 sequence is GFTFSNHDLN (SEQ ID NO: 1) or a variant thereof, the heavy chain CDR2 sequence is YISSASGLISYADAVRG (SEQ ID NO: 2) or a variant thereof; and the heavy chain CDR3 sequence is DPAYTGLYALDF (SEQ ID NO: 3) or a variant thereof, or DPPYSGLYALDF (SEQ ID NO: 4) or a variant thereof; and
(ii) a light chain variable region comprising light chain CDR1, CDR2, and CDR3 sequences, wherein the light chain CDR1 sequence is TLSSELSWYTIV (SEQ ID NO: 5) or a variant thereof, the light chain CDR2 sequence is LKSDGSHSKGD (SEQ ID NO: 6) or a variant thereof, and the light chain CDR3 sequence is CGAGYTLAGQYGWV (SEQ ID NO: 7) or a variant thereof.

88. The method of claim 87, wherein the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

89. The method of claim 88, wherein the variable region FW sequences are human.

90. The method of any one of claims 82-89, wherein the TNFRSF25 agonistic antibody or antigen binding fragment thereof further comprises human heavy chain and light chain constant regions.

91. The method of claim 90, wherein the constant regions are selected from the group consisting of human IgG1, IgG2, IgG3, and IgG4.

92. The method of claim 91, wherein the constant regions are IgG1.

93. The method of claim 91, wherein the constant regions are IgG4.

94. The method of any one of claims 82-93, wherein the TNFRSF25 agonistic antibody or antigen binding fragment thereof comprises a heavy chain variable region of the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of about 85% to about 99% identity thereto.

95. The method of any one of claims 82-94, wherein the TNFRSF25 agonistic antibody or antigen binding fragment thereof comprises a light chain variable region of the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of about 85% to about 99% identity thereto.

96. The method of any one of claims 82-95, wherein the biological adjuvant comprises a secretable vaccine protein.

97. The method of claim 96, wherein the secretable vaccine protein is gp96.

98. The method of claim 96 or claim 97, wherein the secretable vaccine protein is a gp96-Ig fusion protein.

99. The method of claim 98, wherein the gp96-Ig fusion protein lacks the gp96 KDEL (SEQ ID NO: 10) sequence.

100. The method of claim 98 or claim 99, wherein the Ig tag in the gp96-Ig fusion protein comprises the Fc region of human IgG1, IgG2, IgG3, IgG4, IgM, IgA, or IgE.

101. The method of any one of claims 96-100, wherein the biological adjuvant further comprises a T cell costimulatory fusion protein which enhances activation of antigen-specific T cells.

102. The method of claim 101, wherein the T cell costimulatory fusion protein is selected from OX40L-Ig, or a portion thereof that binds to OX40, ICOSL-Ig, or a portion thereof that binds to ICOS, 4 1BBL-Ig, or a portion thereof that binds to 4-1BBR, TL1A-Ig, or a portion thereof that binds to TNFRSF25, GITRL-Ig, or a portion thereof that binds to GITR, CD40L-Ig, or a portion thereof that binds to CD40, and, CD70-Ig, or a portion thereof that binds to CD27.

103. The method of claim 102, wherein the T cell costimulatory fusion protein is an Ig fusion protein.

104. The method of any one of claims 101-103, wherein the Ig tag in the T cell costimulatory fusion protein comprises the Fc region of human IgG1, IgG2, IgG3, IgG4, IgM, IgA, or IgE.

105. The method of claim 103 or claim 104, wherein the T cell costimulatory fusion protein is OX40L-Ig administered in combination with the gp96-Ig fusion protein lacking the gp96 KDEL (SEQ ID NO: 10) sequence.

106. The method of any one of claims 82-105, wherein the method results in an increase in a number of antigen-specific CD8 T cells in the patient.

107. The method of any one of claims 82-106, wherein the method results in an increase in a number of tumor-infiltrating lymphocytes (TILs) in the patient in the patient.

108. The method of any one of claims 82-107, wherein the method results in a decrease in at least one of a tumor size and a tumor growth rate in the patient in the patient.


SEQUENCE LISTING (10 sequences)

SEQ ID NO: 1 - protein, 10 aa, Artificial Sequence
  Synthetic Sequence
GFTFSNHDLN

SEQ ID NO: 2 - protein, 17 aa, Artificial Sequence
  Synthetic Sequence
YISSASGLISYADAVRG

SEQ ID NO: 3 - protein, 12 aa, Artificial Sequence
  Synthetic Sequence
DPAYTGLYALDF

SEQ ID NO: 4 - protein, 12 aa, Artificial Sequence
  Synthetic Sequence
DPPYSGLYALDF

SEQ ID NO: 5 - protein, 12 aa, Artificial Sequence
  Synthetic Sequence
TLSSELSWYTIV

SEQ ID NO: 6 - protein, 11 aa, Artificial Sequence
  Synthetic Sequence
LKSDGSHSKGD

SEQ ID NO: 7 - protein, 14 aa, Artificial Sequence
  Synthetic Sequence
CGAGYTLAGQYGWV

SEQ ID NO: 8 - protein, 121 aa, Artificial Sequence
  Synthetic Sequence
EVQLVESGGGLSQPGNSLQLSCEASGFTFSNHDLNWVRQAPGKGLEWVAYISSASGLISYADAVRGRFTISRDNAKNSLFLQMNNLKSEDTAMYYCARDPPYSGLYALDFWGQGTQVTVSS

SEQ ID NO: 9 - protein, 115 aa, Artificial Sequence
  Synthetic Sequence
QPVLTQSPSASASLSGSVKLTCTLSSELSSYTIVWYQQRPDKAPKYVMYLKSDGSHSKGDGIPDRFSGSSSGAHRYLSISNVQSEDDATYFCGAGYTLAGQYGWVFGSGTKVTVL

SEQ ID NO: 10 - protein, 4 aa, Artificial Sequence
  Synthetic Sequence
KDEL